SEQUENCE LISTING (158 sequences)

SEQ ID NO: 1 - DNA, 63 bp, synthetic construct
  misc_feature 1..63: note = human CD8 signal peptide (DNA)
  source 1..63: mol_type = other DNA
atggctctgcctgtgaccgctctgctgctgcctctggctctgctgctgcatgccgcaagacct

SEQ ID NO: 2 - protein, 21 aa, synthetic construct
  REGION 1..21: note = human CD8 signal peptide (PRT)
MALPVTALLLPLALLLHAARP

SEQ ID NO: 3 - DNA, 135 bp, synthetic construct
  misc_feature 1..135: note = human CD8 hinge region (DNA)
  source 1..135: mol_type = other DNA
accacgacgccagcgccgcggccgccaacaccggcgcccaccatcgcgtcgcagcccctgtccctgcgcccagaggcgtgccggccagcggcggggggcgcagtgcacacgagggggctggacttcgcctgtgat

SEQ ID NO: 4 - protein, 45 aa, synthetic construct
  REGION 1..45: note = human CD8 hinge region (PRT)
TTTPAPRPPTPAPTIASQPLSLRPEACRPAAGGAVHTRGLDFACD

SEQ ID NO: 5 - DNA, 72 bp, synthetic construct
  misc_feature 1..72: note = human CD8 transmembrane region (DNA)
  source 1..72: mol_type = other DNA
atctacatctgggcgcccttggccgggacttgtggggtccttctcctgtcactggttatcaccctttactgc

SEQ ID NO: 6 - protein, 24 aa, synthetic construct
  REGION 1..24: note = human CD8 transmembrane region (PRT)
IYIWAPLAGTCGVLLLSLVITLYC

SEQ ID NO: 7 - DNA, 126 bp, synthetic construct
  misc_feature 1..126: note = human 4-1BB intracellular domain (DNA)
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 8 - protein, 42 aa, synthetic construct
  REGION 1..42: note = human 4-1BB intracellular domain (PRT)
KRGRKKLLYIFKQPFMRPVQTTQEEDGCSCRFPEEEEGGCEL

SEQ ID NO: 9 - DNA, 78 bp, synthetic construct
  misc_feature 1..78: note = Furin T2A cleavage peptide (DNA)
  source 1..78: mol_type = other DNA
cgtagaaagcgttcgggatcgggagagggcagaggaagtcttctaacatgcggtgacgtggaggagaatcccggccct

SEQ ID NO: 10 - protein, 26 aa, synthetic construct
  REGION 1..26: note = Furin T2A cleavage peptide (PRT)
RRKRSGSGEGRGSLLTCGDVEENPGP

SEQ ID NO: 11 - DNA, 117 bp, synthetic construct
  misc_feature 1..117: note = human CD28 hinge region (DNA)
  source 1..117: mol_type = other DNA
atcgaggtgatgtacccccctccctacctggacaacgagaagagcaacggcaccatcatccacgtgaagggcaagcacctgtgccctagccccctgttccccggcccctccaaaccc

SEQ ID NO: 12 - protein, 39 aa, synthetic construct
  REGION 1..39: note = human CD28 hinge region (PRT)
IEVMYPPPYLDNEKSNGTIIHVKGKHLCPSPLFPGPSKP

SEQ ID NO: 13 - DNA, 81 bp, synthetic construct
  misc_feature 1..81: note = human CD28 transmembrane region (DNA)
  source 1..81: mol_type = other DNA
ttctgggtgctcgtcgtggtgggcggcgtgctggcctgctacagcctgctggtgaccgtggcattcatcatcttctgggtg

SEQ ID NO: 14 - protein, 27 aa, synthetic construct
  REGION 1..27: note = human CD28 transmembrane region (PRT)
FWVLVVVGGVLACYSLLVTVAFIIFWV

SEQ ID NO: 15 - DNA, 336 bp, synthetic construct
  misc_feature 1..336: note = human CD3zeta intracellular domain (DNA)
  source 1..336: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 16 - protein, 112 aa, synthetic construct
  REGION 1..112: note = human CD3zeta intracellular domain (PRT)
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - DNA, 1248 bp, synthetic construct
  misc_feature 1..1248: note = Ori (DNA)
  source 1..1248: mol_type = other DNA
gagggcagaggaagtcttctaacatgcggtgacgtggaggagaatcccggccctatgggggccgtcctgaggagcctcctggcctgcagcttctgtgtgctcctgagagcggagcagaaactcatctctgaagaggatctggaacctccaacatgttctcctcagcagtttacttgtttcacgggggaaattgactgtatccctgtggcttggcggtgcgatgggtttactgaatgtgaagaccacagtgatgaactcaattgtcctgtatgctcagagtcccagttccagtgtgccagtgggcagtgtattgatggtgccctccgatgcaatggagatgcaaactgccaggacaaatcagatgagaagaactgtgaagtgctttgtttaattgatcagttccgctgtgccaatggtcagtgcattggaaagcacaagaagtgtgatcataatgtggattgcagtgacaagtcagatgaactggattgttatccgactgaagaaccagcaccacaggccaccaatacagttggttctgttattggcgtaattgtcaccatttttgtgtctggaactgtatactttatctgccagaggatgttgtgtccacgtatgaagggagatggggaaactatgactaatgactatgtagttcatggaccagcttctgtgcctcttggttatgtgccacacccaagttctttgtcaggatctcttccaggaatgtctcgaggtaaatcaatgatcagctccctcagtatcatggggggaagcagtggacccccctatgaccgagcccatgttacaggagcatcatcaagtagttcttcaagcaccaaaggcacttacttccctgcaattttgaaccctccaccatccccagccacagagcgatcacattacactatggaatttggatattcttcaaacagtccttccactcataggtcatacagctacaggccatatagctaccggcactttgcaccccccaccacaccctgcagcacagatgtttgtgacagtgactatgctcctagtcggagaatgacctcagtggcaacagccaagggctataccagtgacttgaactatgattcagaacctgtgcccccacctcccacaccccgaagccaatacttgtcagcagaggagaactatgaaagctgcccaccttctccatacacagagaggagctattctcatcacctctacccaccgccaccctctccctgtacagactcctcc

SEQ ID NO: 18 - protein, 416 aa, synthetic construct
  REGION 1..416: note = Ori (PRT)
EGRGSLLTCGDVEENPGPMGAVLRSLLACSFCVLLRAEQKLISEEDLEPPTCSPQQFTCFTGEIDCIPVAWRCDGFTECEDHSDELNCPVCSESQFQCASGQCIDGALRCNGDANCQDKSDEKNCEVLCLIDQFRCANGQCIGKHKKCDHNVDCSDKSDELDCYPTEEPAPQATNTVGSVIGVIVTIFVSGTVYFICQRMLCPRMKGDGETMTNDYVVHGPASVPLGYVPHPSSLSGSLPGMSRGKSMISSLSIMGGSSGPPYDRAHVTGASSSSSSSTKGTYFPAILNPPPSPATERSHYTMEFGYSSNSPSTHRSYSYRPYSYRHFAPPTTPCSTDVCDSDYAPSRRMTSVATAKGYTSDLNYDSEPVPPPPTPRSQYLSAEENYESCPPSPYTERSYSHHLYPPPPSPCTDSS

SEQ ID NO: 19 - DNA, 15 bp, synthetic construct
  misc_feature 1..15: note = 5E6 HCDR1 (DNA)
  source 1..15: mol_type = other DNA
aactatgtgatgaac

SEQ ID NO: 20 - protein, 5 aa, synthetic construct
  REGION 1..5: note = 5E6 HCDR1; 5E5 HCDR1
NYVMN

SEQ ID NO: 21 - DNA, 51 bp, synthetic construct
  misc_feature 1..51: note = 5E6 HCDR2 (DNA)
  source 1..51: mol_type = other DNA
tatattaacccatataatgatggcaccaaatataatgaaagatttaaaggc

SEQ ID NO: 22 - protein, 17 aa, synthetic construct
  REGION 1..17: note = 5E6 HCDR2; 5E5 HCDR2
YINPYNDGTKYNERFKG

SEQ ID NO: 23 - DNA, 27 bp, synthetic construct
  misc_feature 1..27: note = 5E6 HCDR3 (DNA)
  source 1..27: mol_type = other DNA
ctgtatagaggcaatgccatggattat

SEQ ID NO: 24 - protein, 9 aa, synthetic construct
  REGION 1..9: note = 5E6 HCDR3; 5E5 HCDR3
LYRGNAMDY

SEQ ID NO: 25 - DNA, 354 bp, synthetic construct
  misc_feature 1..354: note = 5E6 VH (DNA)
  source 1..354: mol_type = other DNA
caagtgcagctggtgcagagtggtgcagaagtgaaaaaacctggtgcaagtgtgaaagtgagctgcaaagcaagtggctatacctttaccaactatgtgatgaactgggtgagacaagcccctggtcagagactggaatggatgggctatattaacccatataatgatggcaccaaatataatgaaagatttaaaggcagagtcaccatcactagagatacaagtgcaagcactgcctatatggaactgagcagcctgagaagtgaagatactgcagtgtattattgtgcaagactgtatagaggcaatgccatggattattggggccaaggcaccctggtgactgtgagcagc

SEQ ID NO: 26 - protein, 118 aa, synthetic construct
  REGION 1..118: note = 5E6 VH
QVQLVQSGAEVKKPGASVKVSCKASGYTFTNYVMNWVRQAPGQRLEWMGYINPYNDGTKYNERFKGRVTITRDTSASTAYMELSSLRSEDTAVYYCARLYRGNAMDYWGQGTLVTVSS

SEQ ID NO: 27 - DNA, 51 bp, synthetic construct
  misc_feature 1..51: note = 5E6 LCDR1 (DNA)
  source 1..51: mol_type = other DNA
aaaagcagtcagagcctgctggccagtggcaatcagaaaaactatctgacc

SEQ ID NO: 28 - protein, 17 aa, synthetic construct
  REGION 1..17: note = 5E6 LCDR1
KSSQSLLASGNQKNYLT

SEQ ID NO: 29 - DNA, 21 bp, synthetic construct
  misc_feature 1..21: note = 5E6 LCDR2 (DNA)
  source 1..21: mol_type = other DNA
tgggcaagcactagagaaagt

SEQ ID NO: 30 - protein, 7 aa, synthetic construct
  REGION 1..7: note = 5E6 LCDR2; 5E5 LCDR2; 3A6 LCDR2; 7E3 LCDR2; 17B10 LCDR2; standard LCDR2
WASTRES

SEQ ID NO: 31 - DNA, 27 bp, synthetic construct
  misc_feature 1..27: note = 5E6 LCDR3 (DNA)
  source 1..27: mol_type = other DNA
cagaatgtgtatatttatccatttacc

SEQ ID NO: 32 - protein, 9 aa, synthetic construct
  REGION 1..9: note = 5E6 LCDR3; 5E5 LCDR3; 7E3 LCDR3
QNVYIYPFT

SEQ ID NO: 33 - DNA, 342 bp, synthetic construct
  misc_feature 1..342: note = 5E6 VL (DNA)
  source 1..342: mol_type = other DNA
gatattgtgatgactcagagccctgatagcctggcagtgagcctgggtgaaagagccaccattaactgcaaaagcagtcagagcctgctggccagtggcaatcagaaaaactatctgacctggtatcagcagaaacctggtcagccaccaaaactgctgatttattgggcaagcactagagaaagtggtgtgcctgatagatttagtggcagtggcagtggcactgattttaccctgaccattagcagcctgcaagcagaagatgtggcagtgtattattgtcagaatgtgtatatttatccatttacctttggccaaggcaccaaactggaaattaaaaga

SEQ ID NO: 34 - protein, 114 aa, synthetic construct
  REGION 1..114: note = 5E6 VL
DIVMTQSPDSLAVSLGERATINCKSSQSLLASGNQKNYLTWYQQKPGQPPKLLIYWASTRESGVPDRFSGSGSGTDFTLTISSLQAEDVAVYYCQNVYIYPFTFGQGTKLEIKR

SEQ ID NO: 35 - DNA, 54 bp, synthetic construct
  misc_feature 1..54: note = linker of 5E6 scFv (DNA)
  source 1..54: mol_type = other DNA
gcctccaccggtggcggtggaagcggcggtggcggaagcggcggtggcggcagc

SEQ ID NO: 36 - protein, 18 aa, synthetic construct
  REGION 1..18: note = linker of 5E6 scFv
ASTGGGGSGGGGSGGGGS

SEQ ID NO: 37 - DNA, 750 bp, synthetic construct
  misc_feature 1..750: note = 5E6 scFv (DNA)
  source 1..750: mol_type = other DNA
caagtgcagctggtgcagagtggtgcagaagtgaaaaaacctggtgcaagtgtgaaagtgagctgcaaagcaagtggctatacctttaccaactatgtgatgaactgggtgagacaagcccctggtcagagactggaatggatgggctatattaacccatataatgatggcaccaaatataatgaaagatttaaaggcagagtcaccatcactagagatacaagtgcaagcactgcctatatggaactgagcagcctgagaagtgaagatactgcagtgtattattgtgcaagactgtatagaggcaatgccatggattattggggccaaggcaccctggtgactgtgagcagcgcctccaccggtggcggtggaagcggcggtggcggaagcggcggtggcggcagcgatattgtgatgactcagagccctgatagcctggcagtgagcctgggtgaaagagccaccattaactgcaaaagcagtcagagcctgctggccagtggcaatcagaaaaactatctgacctggtatcagcagaaacctggtcagccaccaaaactgctgatttattgggcaagcactagagaaagtggtgtgcctgatagatttagtggcagtggcagtggcactgattttaccctgaccattagcagcctgcaagcagaagatgtggcagtgtattattgtcagaatgtgtatatttatccatttacctttggccaaggcaccaaactggaaattaaaaga

SEQ ID NO: 38 - protein, 250 aa, synthetic construct
  REGION 1..250: note = 5E6 scFv
QVQLVQSGAEVKKPGASVKVSCKASGYTFTNYVMNWVRQAPGQRLEWMGYINPYNDGTKYNERFKGRVTITRDTSASTAYMELSSLRSEDTAVYYCARLYRGNAMDYWGQGTLVTVSSASTGGGGSGGGGSGGGGSDIVMTQSPDSLAVSLGERATINCKSSQSLLASGNQKNYLTWYQQKPGQPPKLLIYWASTRESGVPDRFSGSGSGTDFTLTISSLQAEDVAVYYCQNVYIYPFTFGQGTKLEIKR

SEQ ID NO: 39 - DNA, 15 bp, synthetic construct
  misc_feature 1..15: note = 5E5 HCDR1 (DNA)
  source 1..15: mol_type = other DNA
aactatgttatgaac

SEQ ID NO: 40 - DNA, 51 bp, synthetic construct
  misc_feature 1..51: note = 5E5 HCDR2 (DNA)
  source 1..51: mol_type = other DNA
tatattaatccttacaatgatggtactaagtacaatgagaggttcaaaggc

SEQ ID NO: 41 - DNA, 27 bp, synthetic construct
  misc_feature 1..27: note = 5E5 HCDR3 (DNA)
  source 1..27: mol_type = other DNA
ctatatagaggcaatgctatggactac

SEQ ID NO: 42 - DNA, 354 bp, synthetic construct
  misc_feature 1..354: note = 5E5 VH (DNA)
  source 1..354: mol_type = other DNA
gaggtccagctgcagcagtctggacctgagctggtaaagcctggggcttcagtgaagatgtcctgcaaggcttctggatacacattcactaactatgttatgaactgggtgaagcagaagcctgggcagggccttgagtggattggatatattaatccttacaatgatggtactaagtacaatgagaggttcaaaggcaaggccacactgacttcagacaaatcctccagcacagccttcatggaggtcagcagcctgacctctgaggactctgcggtctactactgtgcaagactatatagaggcaatgctatggactactggggtcaaggaacctcggtcaccgtctcctca

SEQ ID NO: 43 - protein, 118 aa, synthetic construct
  REGION 1..118: note = 5E5 VH
EVQLQQSGPELVKPGASVKMSCKASGYTFTNYVMNWVKQKPGQGLEWIGYINPYNDGTKYNERFKGKATLTSDKSSSTAFMEVSSLTSEDSAVYYCARLYRGNAMDYWGQGTSVTVSS

SEQ ID NO: 44 - DNA, 51 bp, synthetic construct
  misc_feature 1..51: note = 5E5 LCDR1 (DNA); 3A6 LCDR1 (DNA); 7E3 LCDR1 (DNA); 17B10 LCDR1 (DNA)
  source 1..51: mol_type = other DNA
aagtccagtcagagtctgttaaacagtggaaatcaaaagaactacttgacc

SEQ ID NO: 45 - protein, 17 aa, synthetic construct
  REGION 1..17: note = 5E5 LCDR1; 3A6 LCDR1; 7E3 LCDR1; 17B10 LCDR1
KSSQSLLNSGNQKNYLT

SEQ ID NO: 46 - DNA, 21 bp, synthetic construct
  misc_feature 1..21: note = 5E5 LCDR2 (DNA); 3A6 LCDR2 (DNA); 7E3 LCDR2 (DNA); 17B10 LCDR2 (DNA)
  source 1..21: mol_type = other DNA
tgggcatccactagggaatct

SEQ ID NO: 47 - DNA, 27 bp, synthetic construct
  misc_feature 1..27: note = 5E5 LCDR3 (DNA); 7E3 LCDR3 (DNA)
  source 1..27: mol_type = other DNA
cagaatgtttatatttatccgttcacg

SEQ ID NO: 48 - DNA, 342 bp, synthetic construct
  misc_feature 1..342: note = 5E5 VL (DNA); 7E3 VL (DNA)
  source 1..342: mol_type = other DNA
gacattgtgatgacacagtctccatcctccctgactgtgacagtaagagagaaggtcactttgagctgcaagtccagtcagagtctgttaaacagtggaaatcaaaagaactacttgacctggtaccagcagaaaccagggcagcctcctaaacttttgatctactgggcatccactagggaatctggggtccctgatcgcttcacaggcagtggatctggcacagatttcactctcaccatcagcagtgtgcaggctgaagacctggcagtttattactgtcagaatgtttatatttatccgttcacgttcggtgctgggaccaagctggagctgaaacgg

SEQ ID NO: 49 - protein, 114 aa, synthetic construct
  REGION 1..114: note = 5E5 VL; 7E3 VL
DIVMTQSPSSLTVTVREKVTLSCKSSQSLLNSGNQKNYLTWYQQKPGQPPKLLIYWASTRESGVPDRFTGSGSGTDFTLTISSVQAEDLAVYYCQNVYIYPFTFGAGTKLELKR

SEQ ID NO: 50 - DNA, 45 bp, synthetic construct
  misc_feature 1..45: note = linker of 5E5, 3A6, 7E3, 14E12, 17B10 scFv (DNA)
  source 1..45: mol_type = other DNA
ggcggaggaggatccggaggcggaggaagcggaggaggcggatct

SEQ ID NO: 51 - protein, 15 aa, synthetic construct
  REGION 1..15: note = linker of 5E5, 3A6, 7E3, 14E12, 17B10, standard scFv
GGGGSGGGGSGGGGS

SEQ ID NO: 52 - DNA, 741 bp, synthetic construct
  misc_feature 1..741: note = 5E5 scFv (DNA)
  source 1..741: mol_type = other DNA
gacattgtgatgacacagtctccatcctccctgactgtgacagtaagagagaaggtcactttgagctgcaagtccagtcagagtctgttaaacagtggaaatcaaaagaactacttgacctggtaccagcagaaaccagggcagcctcctaaacttttgatctactgggcatccactagggaatctggggtccctgatcgcttcacaggcagtggatctggcacagatttcactctcaccatcagcagtgtgcaggctgaagacctggcagtttattactgtcagaatgtttatatttatccgttcacgttcggtgctgggaccaagctggagctgaaacggggcggaggaggatccggaggcggaggaagcggaggaggcggatctgaggtccagctgcagcagtctggacctgagctggtaaagcctggggcttcagtgaagatgtcctgcaaggcttctggatacacattcactaactatgttatgaactgggtgaagcagaagcctgggcagggccttgagtggattggatatattaatccttacaatgatggtactaagtacaatgagaggttcaaaggcaaggccacactgacttcagacaaatcctccagcacagccttcatggaggtcagcagcctgacctctgaggactctgcggtctactactgtgcaagactatatagaggcaatgctatggactactggggtcaaggaacctcggtcaccgtctcctca

SEQ ID NO: 53 - protein, 247 aa, synthetic construct
  REGION 1..247: note = 5E5 scFv
DIVMTQSPSSLTVTVREKVTLSCKSSQSLLNSGNQKNYLTWYQQKPGQPPKLLIYWASTRESGVPDRFTGSGSGTDFTLTISSVQAEDLAVYYCQNVYIYPFTFGAGTKLELKRGGGGSGGGGSGGGGSEVQLQQSGPELVKPGASVKMSCKASGYTFTNYVMNWVKQKPGQGLEWIGYINPYNDGTKYNERFKGKATLTSDKSSSTAFMEVSSLTSEDSAVYYCARLYRGNAMDYWGQGTSVTVSS

SEQ ID NO: 54 - DNA, 15 bp, synthetic construct
  misc_feature 1..15: note = 3A6 HCDR1 (DNA)
  source 1..15: mol_type = other DNA
cgttatggcatgtct

SEQ ID NO: 55 - protein, 5 aa, synthetic construct
  REGION 1..5: note = 3A6 HCDR1
RYGMS

SEQ ID NO: 56 - DNA, 51 bp, synthetic construct
  misc_feature 1..51: note = 3A6 HCDR2 (DNA)
  source 1..51: mol_type = other DNA
accattattagtggtggtattaacacctactattcagacagtgtgaagggg

SEQ ID NO: 57 - protein, 17 aa, synthetic construct
  REGION 1..17: note = 3A6 HCDR2
TIISGGINTYYSDSVKG

SEQ ID NO: 58 - DNA, 27 bp, synthetic construct
  misc_feature 1..27: note = 3A6 HCDR3 (DNA)
  source 1..27: mol_type = other DNA
ctctactatggcaatgctatggactac

SEQ ID NO: 59 - protein, 9 aa, synthetic construct
  REGION 1..9: note = 3A6 HCDR3
LYYGNAMDY

SEQ ID NO: 60 - DNA, 354 bp, synthetic construct
  misc_feature 1..354: note = 3A6 VH (DNA)
  source 1..354: mol_type = other DNA
gaggtgcagttggtggagtctgggggagacttagtgcagcctggagggtccctgaaactctcctgtgcagcctctggattcactttaagtcgttatggcatgtcttgggttcgccagactcctgacaagaggctggagtgggtctcaaccattattagtggtggtattaacacctactattcagacagtgtgaaggggcgattctccatctccagagacaatgccaggaacaccctgtacctgcaaatgagcagtctgaagtctgaggacacagccatgtattactgtggaagactctactatggcaatgctatggactactgggggcaaggaaccgctgtcaccgtctcctca

SEQ ID NO: 61 - protein, 118 aa, synthetic construct
  REGION 1..118: note = 3A6 VH
EVQLVESGGDLVQPGGSLKLSCAASGFTLSRYGMSWVRQTPDKRLEWVSTIISGGINTYYSDSVKGRFSISRDNARNTLYLQMSSLKSEDTAMYYCGRLYYGNAMDYWGQGTAVTVSS

SEQ ID NO: 62 - DNA, 27 bp, synthetic construct
  misc_feature 1..27: note = 3A6 LCDR3 (DNA)
  source 1..27: mol_type = other DNA
cagaatgattatagttatcctctcacg

SEQ ID NO: 63 - protein, 9 aa, synthetic construct
  REGION 1..9: note = 3A6 LCDR3
QNDYSYPLT

SEQ ID NO: 64 - DNA, 342 bp, synthetic construct
  misc_feature 1..342: note = 3A6 VL (DNA)
  source 1..342: mol_type = other DNA
gacattgtgatgacacagtctccatcctccctgactgtgacagcaggagagaaggtcactatgagctgcaagtccagtcagagtctgttaaacagtggaaatcaaaagaactacttgacctggtaccagcagaaaccagggcagcctcctaaactgttgatctactgggcatccactagggaatctggggtccctgatcgcttcacaggcagtggatctggaacacatttcactctcaccatcagcagtgtgcaggctgaagacctggcagtttattactgtcagaatgattatagttatcctctcacgttcggtgctgggaccaagctggagctgaaacgg

SEQ ID NO: 65 - protein, 114 aa, synthetic construct
  REGION 1..114: note = 3A6 VL
DIVMTQSPSSLTVTAGEKVTMSCKSSQSLLNSGNQKNYLTWYQQKPGQPPKLLIYWASTRESGVPDRFTGSGSGTHFTLTISSVQAEDLAVYYCQNDYSYPLTFGAGTKLELKR

SEQ ID NO: 66 - DNA, 741 bp, synthetic construct
  misc_feature 1..741: note = 3A6 scFv (DNA)
  source 1..741: mol_type = other DNA
gacattgtgatgacacagtctccatcctccctgactgtgacagcaggagagaaggtcactatgagctgcaagtccagtcagagtctgttaaacagtggaaatcaaaagaactacttgacctggtaccagcagaaaccagggcagcctcctaaactgttgatctactgggcatccactagggaatctggggtccctgatcgcttcacaggcagtggatctggaacacatttcactctcaccatcagcagtgtgcaggctgaagacctggcagtttattactgtcagaatgattatagttatcctctcacgttcggtgctgggaccaagctggagctgaaacggggcggaggaggatccggaggcggaggaagcggaggaggcggatctgaggtgcagttggtggagtctgggggagacttagtgcagcctggagggtccctgaaactctcctgtgcagcctctggattcactttaagtcgttatggcatgtcttgggttcgccagactcctgacaagaggctggagtgggtctcaaccattattagtggtggtattaacacctactattcagacagtgtgaaggggcgattctccatctccagagacaatgccaggaacaccctgtacctgcaaatgagcagtctgaagtctgaggacacagccatgtattactgtggaagactctactatggcaatgctatggactactgggggcaaggaaccgctgtcaccgtctcctca

SEQ ID NO: 67 - protein, 247 aa, synthetic construct
  REGION 1..247: note = 3A6 scFv
DIVMTQSPSSLTVTAGEKVTMSCKSSQSLLNSGNQKNYLTWYQQKPGQPPKLLIYWASTRESGVPDRFTGSGSGTHFTLTISSVQAEDLAVYYCQNDYSYPLTFGAGTKLELKRGGGGSGGGGSGGGGSEVQLVESGGDLVQPGGSLKLSCAASGFTLSRYGMSWVRQTPDKRLEWVSTIISGGINTYYSDSVKGRFSISRDNARNTLYLQMSSLKSEDTAMYYCGRLYYGNAMDYWGQGTAVTVSS

SEQ ID NO: 68 - DNA, 15 bp, synthetic construct
  misc_feature 1..15: note = 7E3 HCDR1 (DNA); 14E12 HCDR1 (DNA); 17B10 HCDR1 (DNA)
  source 1..15: mol_type = other DNA
aactatattatacac

SEQ ID NO: 69 - protein, 5 aa, synthetic construct
  REGION 1..5: note = 7E3 HCDR1; 14E12 HCDR1; 17B10 HCDR1
NYIIH

SEQ ID NO: 70 - DNA, 51 bp, synthetic construct
  misc_feature 1..51: note = 7E3 HCDR2 (DNA); 14E12 HCDR2 (DNA); 17B10 HCDR2 (DNA)
  source 1..51: mol_type = other DNA
tatattaatccttacactgatggtcctaagtacaatgagaagttcaaaggc

SEQ ID NO: 71 - protein, 17 aa, synthetic construct
  REGION 1..17: note = 7E3 HCDR2; 14E12 HCDR2; 17B10 HCDR2
YINPYTDGPKYNEKFKG

SEQ ID NO: 72 - DNA, 27 bp, synthetic construct
  misc_feature 1..27: note = 7E3 HCDR3 (DNA); 14E12 HCDR3 (DNA); 17B10 HCDR3 (DNA)
  source 1..27: mol_type = other DNA
ctaaatagaggcaatgctatggactac

SEQ ID NO: 73 - protein, 9 aa, synthetic construct
  REGION 1..9: note = 7E3 HCDR3; 14E12 HCDR3; 17B10 HCDR3
LNRGNAMDY

SEQ ID NO: 74 - DNA, 354 bp, synthetic construct
  misc_feature 1..354: note = 7E3 VH (DNA)
  source 1..354: mol_type = other DNA
gaggtccagttgcagcagtctggacctgagctggtaaagcctggggcttcagtgaagatgtcctgcaaggcttctggatacacattcactaactatattatacactgggtgaagcagaagcctgggcagggccttgagtggattggatatattaatccttacactgatggtcctaagtacaatgagaagttcaaaggcagggccacactgacttcagacaaatcctccagtacagcctacatggagttcagcagcctgacctctgaggactctgcggtctattactgtgcaagactaaatagaggcaatgctatggactactggggtcaaggaacctcagtcaccgtctcctca

SEQ ID NO: 75 - protein, 118 aa, synthetic construct
  REGION 1..118: note = 7E3 VH
EVQLQQSGPELVKPGASVKMSCKASGYTFTNYIIHWVKQKPGQGLEWIGYINPYTDGPKYNEKFKGRATLTSDKSSSTAYMEFSSLTSEDSAVYYCARLNRGNAMDYWGQGTSVTVSS

SEQ ID NO: 76 - DNA, 741 bp, synthetic construct
  misc_feature 1..741: note = 7E3 scFv (DNA)
  source 1..741: mol_type = other DNA
gacattgtgatgacacagtctccatcctccctgactgtgacagtaagagagaaggtcactttgagctgcaagtccagtcagagtctgttaaacagtggaaatcaaaagaactacttgacctggtaccagcagaaaccagggcagcctcctaaacttttgatctactgggcatccactagggaatctggggtccctgatcgcttcacaggcagtggatctggcacagatttcactctcaccatcagcagtgtgcaggctgaagacctggcagtttattactgtcagaatgtttatatttatccgttcacgttcggtgctgggaccaagctggagctgaaacggggcggaggaggatccggaggcggaggaagcggaggaggcggatctgaggtccagttgcagcagtctggacctgagctggtaaagcctggggcttcagtgaagatgtcctgcaaggcttctggatacacattcactaactatattatacactgggtgaagcagaagcctgggcagggccttgagtggattggatatattaatccttacactgatggtcctaagtacaatgagaagttcaaaggcagggccacactgacttcagacaaatcctccagtacagcctacatggagttcagcagcctgacctctgaggactctgcggtctattactgtgcaagactaaatagaggcaatgctatggactactggggtcaaggaacctcagtcaccgtctcctca

SEQ ID NO: 77 - protein, 247 aa, synthetic construct
  REGION 1..247: note = 7E3 scFv
DIVMTQSPSSLTVTVREKVTLSCKSSQSLLNSGNQKNYLTWYQQKPGQPPKLLIYWASTRESGVPDRFTGSGSGTDFTLTISSVQAEDLAVYYCQNVYIYPFTFGAGTKLELKRGGGGSGGGGSGGGGSEVQLQQSGPELVKPGASVKMSCKASGYTFTNYIIHWVKQKPGQGLEWIGYINPYTDGPKYNEKFKGRATLTSDKSSSTAYMEFSSLTSEDSAVYYCARLNRGNAMDYWGQGTSVTVSS

SEQ ID NO: 78 - DNA, 354 bp, synthetic construct
  misc_feature 1..354: note = 14E12 VH (DNA); 17B10 VH (DNA)
  source 1..354: mol_type = other DNA
gaggtgcagctgcaggagtctggacctgagctggtaaagcctggggcttcagtgaagatgtcctgcaaggcttctggatacacattcactaactatattatacactgggtgaagcagaagcctgggcagggccttgagtggattggatatattaatccttacactgatggtcctaagtacaatgagaagttcaaaggcagggccacactgacttcagacaaatcctccagtacagcctacatggagttcagcagcctgacctctgaggactctgcggtctattactgtgcaagactaaatagaggcaatgctatggactactggggtcaaggaacctcagtcaccgtctcctca

SEQ ID NO: 79 - protein, 118 aa, synthetic construct
  REGION 1..118: note = 14E12 VH; 17B10 VH
EVQLQESGPELVKPGASVKMSCKASGYTFTNYIIHWVKQKPGQGLEWIGYINPYTDGPKYNEKFKGRATLTSDKSSSTAYMEFSSLTSEDSAVYYCARLNRGNAMDYWGQGTSVTVSS

SEQ ID NO: 80 - DNA, 51 bp, synthetic construct
  misc_feature 1..51: note = 14E12 LCDR1 (DNA)
  source 1..51: mol_type = other DNA
aagtccagtcagagtctgttaaacaatggaaatcaaaagaactatttggcc

SEQ ID NO: 81 - protein, 17 aa, synthetic construct
  REGION 1..17: note = 14E12 LCDR1
KSSQSLLNNGNQKNYLA

SEQ ID NO: 82 - DNA, 21 bp, synthetic construct
  misc_feature 1..21: note = 14E12 LCDR2 (DNA)
  source 1..21: mol_type = other DNA
ggggcatccactagggaatct

SEQ ID NO: 83 - protein, 7 aa, synthetic construct
  REGION 1..7: note = 14E12 LCDR2
GASTRES

SEQ ID NO: 84 - DNA, 27 bp, synthetic construct
  misc_feature 1..27: note = 14E12 LCDR3 (DNA)
  source 1..27: mol_type = other DNA
cagaatgatctttattatccattcacg

SEQ ID NO: 85 - protein, 9 aa, synthetic construct
  REGION 1..9: note = 14E12 LCDR3
QNDLYYPFT

SEQ ID NO: 86 - DNA, 342 bp, synthetic construct
  misc_feature 1..342: note = 14E12 VL (DNA)
  source 1..342: mol_type = other DNA
gatattgtgatgacccagtctccatcttccctgagtgtgtcagcaggagagaaggtcactatgagctgcaagtccagtcagagtctgttaaacaatggaaatcaaaagaactatttggcctggtaccagcagaaaccagggcagcctcctaaactgttgatctacggggcatccactagggaatctggggtccctgatcgcttcacaggcagtggatctggaaccgatttcgctcttaccatcagcagtgtgcaggctgaagacctggcagtttattactgtcagaatgatctttattatccattcacgttcggctcggggacaaagttggacataaaacgg

SEQ ID NO: 87 - protein, 114 aa, synthetic construct
  REGION 1..114: note = 14E12 VL
DIVMTQSPSSLSVSAGEKVTMSCKSSQSLLNNGNQKNYLAWYQQKPGQPPKLLIYGASTRESGVPDRFTGSGSGTDFALTISSVQAEDLAVYYCQNDLYYPFTFGSGTKLDIKR

SEQ ID NO: 88 - DNA, 741 bp, synthetic construct
  misc_feature 1..741: note = 14E12 scFv (DNA)
  source 1..741: mol_type = other DNA
gatattgtgatgacccagtctccatcttccctgagtgtgtcagcaggagagaaggtcactatgagctgcaagtccagtcagagtctgttaaacaatggaaatcaaaagaactatttggcctggtaccagcagaaaccagggcagcctcctaaactgttgatctacggggcatccactagggaatctggggtccctgatcgcttcacaggcagtggatctggaaccgatttcgctcttaccatcagcagtgtgcaggctgaagacctggcagtttattactgtcagaatgatctttattatccattcacgttcggctcggggacaaagttggacataaaacggggcggaggaggatccggaggcggaggaagcggaggaggcggatctgaggtgcagctgcaggagtctggacctgagctggtaaagcctggggcttcagtgaagatgtcctgcaaggcttctggatacacattcactaactatattatacactgggtgaagcagaagcctgggcagggccttgagtggattggatatattaatccttacactgatggtcctaagtacaatgagaagttcaaaggcagggccacactgacttcagacaaatcctccagtacagcctacatggagttcagcagcctgacctctgaggactctgcggtctattactgtgcaagactaaatagaggcaatgctatggactactggggtcaaggaacctcagtcaccgtctcctca

SEQ ID NO: 89 - protein, 247 aa, synthetic construct
  REGION 1..247: note = 14E12 scFv
DIVMTQSPSSLSVSAGEKVTMSCKSSQSLLNNGNQKNYLAWYQQKPGQPPKLLIYGASTRESGVPDRFTGSGSGTDFALTISSVQAEDLAVYYCQNDLYYPFTFGSGTKLDIKRGGGGSGGGGSGGGGSEVQLQESGPELVKPGASVKMSCKASGYTFTNYIIHWVKQKPGQGLEWIGYINPYTDGPKYNEKFKGRATLTSDKSSSTAYMEFSSLTSEDSAVYYCARLNRGNAMDYWGQGTSVTVSS

SEQ ID NO: 90 - DNA, 27 bp, synthetic construct
  misc_feature 1..27: note = 17B10 LCDR3 (DNA)
  source 1..27: mol_type = other DNA
cagaatgattatagttttccattcacg

SEQ ID NO: 91 - protein, 9 aa, synthetic construct
  REGION 1..9: note = 17B10 LCDR3
QNDYSFPFT

SEQ ID NO: 92 - DNA, 342 bp, synthetic construct
  misc_feature 1..342: note = 17B10 VL (DNA)
  source 1..342: mol_type = other DNA
gatattgtgatgacccagtctccatcctccctgactgtgacagcaggagagaaggtcactatgaactgcaagtccagtcagagtctgttaaacagtggaaatcaaaagaactacttgacctggtaccagcagaaaccagggcagcctcctaaactgttgatctactgggcatccactagggaatctggggtccctgatcggttcacaggcagtggatctggaacagatttcactctcaccatcagcagtgtgcaggctgaagacctggcagtttattactgtcagaatgattatagttttccattcacgttcggctcggggacaaagttggaaataaaacgg

SEQ ID NO: 93 - protein, 114 aa, synthetic construct
  REGION 1..114: note = 17B10 VL
DIVMTQSPSSLTVTAGEKVTMNCKSSQSLLNSGNQKNYLTWYQQKPGQPPKLLIYWASTRESGVPDRFTGSGSGTDFTLTISSVQAEDLAVYYCQNDYSFPFTFGSGTKLEIKR

SEQ ID NO: 94 - DNA, 741 bp, synthetic construct
  misc_feature 1..741: note = 17B10 scFv (DNA)
  source 1..741: mol_type = other DNA
gatattgtgatgacccagtctccatcctccctgactgtgacagcaggagagaaggtcactatgaactgcaagtccagtcagagtctgttaaacagtggaaatcaaaagaactacttgacctggtaccagcagaaaccagggcagcctcctaaactgttgatctactgggcatccactagggaatctggggtccctgatcggttcacaggcagtggatctggaacagatttcactctcaccatcagcagtgtgcaggctgaagacctggcagtttattactgtcagaatgattatagttttccattcacgttcggctcggggacaaagttggaaataaaacggggcggaggaggatccggaggcggaggaagcggaggaggcggatctgaggtgcagctgcaggagtctggacctgagctggtaaagcctggggcttcagtgaagatgtcctgcaaggcttctggatacacattcactaactatattatacactgggtgaagcagaagcctgggcagggccttgagtggattggatatattaatccttacactgatggtcctaagtacaatgagaagttcaaaggcagggccacactgacttcagacaaatcctccagtacagcctacatggagttcagcagcctgacctctgaggactctgcggtctattactgtgcaagactaaatagaggcaatgctatggactactggggtcaaggaacctcagtcaccgtctcctca

SEQ ID NO: 95 - protein, 247 aa, synthetic construct
  REGION 1..247: note = 17B10 scFv
DIVMTQSPSSLTVTAGEKVTMNCKSSQSLLNSGNQKNYLTWYQQKPGQPPKLLIYWASTRESGVPDRFTGSGSGTDFTLTISSVQAEDLAVYYCQNDYSFPFTFGSGTKLEIKRGGGGSGGGGSGGGGSEVQLQESGPELVKPGASVKMSCKASGYTFTNYIIHWVKQKPGQGLEWIGYINPYTDGPKYNEKFKGRATLTSDKSSSTAYMEFSSLTSEDSAVYYCARLNRGNAMDYWGQGTSVTVSS

SEQ ID NO: 96 - DNA, 15 bp, synthetic construct
  misc_feature 1..15: note = MSLN3 HCDR1 (DNA)
  source 1..15: mol_type = other DNA
cgctataacatgggc

SEQ ID NO: 97 - protein, 5 aa, synthetic construct
  REGION 1..5: note = MSLN3 HCDR1
RYNMG

SEQ ID NO: 98 - DNA, 51 bp, synthetic construct
  misc_feature 1..51: note = MSLN3 HCDR2 (DNA)
  source 1..51: mol_type = other DNA
tatattagctggcgcggtggcagcacctattacccagatagcgtggaaggc

SEQ ID NO: 99 - protein, 17 aa, synthetic construct
  REGION 1..17: note = MSLN3 HCDR2
YISWRGGSTYYPDSVEG

SEQ ID NO: 100 - DNA, 36 bp, synthetic construct
  misc_feature 1..36: note = MSLN3 HCDR3 (DNA)
  source 1..36: mol_type = other DNA
agcatcaaaaccgccgatccagatgattacgattac

SEQ ID NO: 101 - protein, 12 aa, synthetic construct
  REGION 1..12: note = MSLN3 HCDR3
SIKTADPDDYDY

SEQ ID NO: 102 - DNA, 363 bp, synthetic construct
  misc_feature 1..363: note = MSLN3 VHH (DNA)
  source 1..363: mol_type = other DNA
gaggtgcagctggtggagagcggtggtggtctcgtgcagcccggcggtagtctgcgcctcagctgtgccgccagcggtaacacccttagccgctataacatgggctggtttcgccaagcccccggcaaaggtcgcgaactggtggcctatattagctggcgcggtggcagcacctattacccagatagcgtggaaggccgcttcaccatcagccgcgataacgccaagcgcatggtgtatctgcagatgaacagtctgcgcgccgaggacaccgccgtgtattattgtgccgccagcatcaaaaccgccgatccagatgattacgattactggggccaaggcacccaagtgaccgtgagcagc

SEQ ID NO: 103 - protein, 121 aa, synthetic construct
  REGION 1..121: note = MSLN3 VHH
EVQLVESGGGLVQPGGSLRLSCAASGNTLSRYNMGWFRQAPGKGRELVAYISWRGGSTYYPDSVEGRFTISRDNAKRMVYLQMNSLRAEDTAVYYCAASIKTADPDDYDYWGQGTQVTVSS

SEQ ID NO: 104 - protein, 5 aa, synthetic construct
  REGION 1..5: note = A06 HCDR1
DYYMG

SEQ ID NO: 105 - protein, 17 aa, synthetic construct
  REGION 1..17: note = A06 HCDR2
GITSSGSDTLYPDSVEG

SEQ ID NO: 106 - protein, 19 aa, synthetic construct
  REGION 1..19: note = A06 HCDR3
TYQGNRPVRTDQFLGGYNY

SEQ ID NO: 107 - protein, 128 aa, synthetic construct
  REGION 1..128: note = A06 VHH
EVQLVESGGGLVQPGGSLRLSCAASGIIFSDYYMGWFRQAPGKGRELVAGITSSGSDTLYPDSVEGRFTISRDNAKRMVYLQMNSLRAEDTAVYYCAATYQGNRPVRTDQFLGGYNYWGQGTLVTVSS

SEQ ID NO: 108 - protein, 5 aa, synthetic construct
  REGION 1..5: note = SS1 HCDR1
GYTMN

SEQ ID NO: 109 - protein, 17 aa, synthetic construct
  REGION 1..17: note = SS1 HCDR2
LITPYNGASSYNQKFRG

SEQ ID NO: 110 - protein, 10 aa, synthetic construct
  REGION 1..10: note = SS1 HCDR3
GGYDGRGFDY

SEQ ID NO: 111 - protein, 119 aa, synthetic construct
  REGION 1..119: note = SS1 VH
QVQLQQSGPELEKPGASVKLSCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSS

SEQ ID NO: 112 - protein, 10 aa, synthetic construct
  REGION 1..10: note = SS1 LCDR1
SASSSVSYMH

SEQ ID NO: 113 - protein, 7 aa, synthetic construct
  REGION 1..7: note = SS1 LCDR2
DTSKLAS

SEQ ID NO: 114 - protein, 9 aa, synthetic construct
  REGION 1..9: note = SS1 LCDR3
QQWSGYPLT

SEQ ID NO: 115 - protein, 107 aa, synthetic construct
  REGION 1..107: note = SS1 VL
DIELTQSPAIMSASPGEKVTMTCSASSSVSYMHWYQQKSGTSPKRWIYDTSKLASGVPGRFSGSGSGNSYSLTISSVEAEDDATYYCQQWSGYPLTFGAGTKLEIKR

SEQ ID NO: 116 - protein, 241 aa, synthetic construct
  REGION 1..241: note = SS1 scFv
QVQLQQSGPELEKPGASVKLSCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGVGGSGGGGSGGGGSDIELTQSPAIMSASPGEKVTMTCSASSSVSYMHWYQQKSGTSPKRWIYDTSKLASGVPGRFSGSGSGNSYSLTISSVEAEDDATYYCQQWSGYPLTFGAGTKLEIKR

SEQ ID NO: 117 - protein, 6 aa, synthetic construct
  REGION 1..6: note = YP218 HCDR1
FYFYAC

SEQ ID NO: 118 - protein, 18 aa, synthetic construct
  REGION 1..18: note = YP218 HCDR2
CIYTAGSGSTYYASWAKG

SEQ ID NO: 119 - protein, 13 aa, synthetic construct
  REGION 1..13: note = YP218 HCDR3
STANTRSTYYLNL

SEQ ID NO: 120 - protein, 123 aa, synthetic construct
  REGION 1..123: note = YP218 VH
QQQLEESGGGLVKPEGSLTLTCKASGFDLGFYFYACWVRQAPGKGLEWIACIYTAGSGSTYYASWAKGRFTISKASSTTVTLQMTSLAAADTATYFCARSTANTRSTYYLNLWGPGTLVTVSS

SEQ ID NO: 121 - protein, 11 aa, synthetic construct
  REGION 1..11: note = YP218 LCDR1
QASQRISSYLS

SEQ ID NO: 122 - protein, 7 aa, synthetic construct
  REGION 1..7: note = YP218 LCDR2
GASTLAS

SEQ ID NO: 123 - protein, 13 aa, synthetic construct
  REGION 1..13: note = YP218 LCDR3
QSYAYFDSNNWHA

SEQ ID NO: 124 - protein, 110 aa, synthetic construct
  REGION 1..110: note = YP218 VL
DVVMTQTPASVSEPVGGTVTIKCQASQRISSYLSWYQQKPGQRPKLLIFGASTLASGVPSRFKGSGSGTEYTLTISDLECADAATYYCQSYAYFDSNNWHAFGGGTEVVV

SEQ ID NO: 125 - protein, 248 aa, synthetic construct
  REGION 1..248: note = YP218 scFv
QQQLEESGGGLVKPEGSLTLTCKASGFDLGFYFYACWVRQAPGKGLEWIACIYTAGSGSTYYASWAKGRFTISKASSTTVTLQMTSLAAADTATYFCARSTANTRSTYYLNLWGPGTLVTVSSGGGGSGGGGSGGGGSDVVMTQTPASVSEPVGGTVTIKCQASQRISSYLSWYQQKPGQRPKLLIFGASTLASGVPSRFKGSGSGTEYTLTISDLECADAATYYCQSYAYFDSNNWHAFGGGTEVVV

SEQ ID NO: 126 - DNA, 18 bp, synthetic construct
  misc_feature 1..18: note = standard HCDR1 (DNA)
  source 1..18: mol_type = other DNA
agcggctacaactggcac

SEQ ID NO: 127 - protein, 6 aa, synthetic construct
  REGION 1..6: note = standard HCDR1
SGYNWH

SEQ ID NO: 128 - DNA, 48 bp, synthetic construct
  misc_feature 1..48: note = standard HCDR2 (DNA)
  source 1..48: mol_type = other DNA
tacatccactacaccggcagcaccaactacaaccccgccctgagaagc

SEQ ID NO: 129 - protein, 16 aa, synthetic construct
  REGION 1..16: note = standard HCDR2
YIHYTGSTNYNPALRS

SEQ ID NO: 130 - DNA, 27 bp, synthetic construct
  misc_feature 1..27: note = standard HCDR3 (DNA)
  source 1..27: mol_type = other DNA
atctacaacggcaacagcttcccttat

SEQ ID NO: 131 - protein, 9 aa, synthetic construct
  REGION 1..9: note = standard HCDR3
IYNGNSFPY

SEQ ID NO: 132 - DNA, 354 bp, synthetic construct
  misc_feature 1..354: note = standard VH (DNA)
  source 1..354: mol_type = other DNA
caggtgcaactacaggagagcggccccggtctgatcaagcccagccagaccctgagcctgacctgcaccgtgagcggcggcagcatcagcagcggctacaactggcactggatcagacagccccccggcaagggcctggagtggatcggctacatccactacaccggcagcaccaactacaaccccgccctgagaagcagagtgaccatcagcgtggacaccagcaagaaccagttcagcctgaagctgagcagcgtgaccgccgccgacaccgccatctactactgcgccagaatctacaacggcaacagcttcccttattggggccagggcaccaccgtgaccgtgagcagc

SEQ ID NO: 133 - protein, 118 aa, synthetic construct
  REGION 1..118: note = standard VH
QVQLQESGPGLIKPSQTLSLTCTVSGGSISSGYNWHWIRQPPGKGLEWIGYIHYTGSTNYNPALRSRVTISVDTSKNQFSLKLSSVTAADTAIYYCARIYNGNSFPYWGQGTTVTVSS

SEQ ID NO: 134 - DNA, 51 bp, synthetic construct
  misc_feature 1..51: note = standard LCDR1 (DNA)
  source 1..51: mol_type = other DNA
aagagcagccagagcctgttcaacagcggcaaccagaagaactacctgacc

SEQ ID NO: 135 - protein, 17 aa, synthetic construct
  REGION 1..17: note = standard LCDR1
KSSQSLFNSGNQKNYLT

SEQ ID NO: 136 - DNA, 21 bp, synthetic construct
  misc_feature 1..21: note = standard LCDR2 (DNA)
  source 1..21: mol_type = other DNA
tgggccagcaccagagagagc

SEQ ID NO: 137 - DNA, 27 bp, synthetic construct
  misc_feature 1..27: note = standard LCDR3 (DNA)
  source 1..27: mol_type = other DNA
cagaacgcctatagctttccctataca

SEQ ID NO: 138 - protein, 9 aa, synthetic construct
  REGION 1..9: note = standard LCDR3
QNAYSFPYT

SEQ ID NO: 139 - DNA, 342 bp, synthetic construct
  misc_feature 1..342: note = standard VL (DNA)
  source 1..342: mol_type = other DNA
gacatcgtgatgacccagagccccgacagcctggccgtgagcctgggcgagagagccaccatcaactgcaagagcagccagagcctgttcaacagcggcaaccagaagaactacctgacctggtaccagcagaagcccggccagccccccaagctgctgatctactgggccagcaccagagagagcggcgtgcccgacagattcagcggcagcggcagcggcaccgacttcaccctgaccatcagcagcctgcaggccgaggacgtggccgtgtactactgccagaacgcctatagctttccctatacatttggcggtgggaccaagctggagatcaagcgg

SEQ ID NO: 140 - protein, 114 aa, synthetic construct
  REGION 1..114: note = standard VL
DIVMTQSPDSLAVSLGERATINCKSSQSLFNSGNQKNYLTWYQQKPGQPPKLLIYWASTRESGVPDRFSGSGSGTDFTLTISSLQAEDVAVYYCQNAYSFPYTFGGGTKLEIKR

SEQ ID NO: 141 - DNA, 45 bp, synthetic construct
  misc_feature 1..45: note = linker of standard scFv (DNA)
  source 1..45: mol_type = other DNA
ggtgggggcggctctggtggtggaggctccggaggcggtgggagc

SEQ ID NO: 142 - DNA, 741 bp, synthetic construct
  misc_feature 1..741: note = standard scFv (DNA)
  source 1..741: mol_type = other DNA
gacatcgtgatgacccagagccccgacagcctggccgtgagcctgggcgagagagccaccatcaactgcaagagcagccagagcctgttcaacagcggcaaccagaagaactacctgacctggtaccagcagaagcccggccagccccccaagctgctgatctactgggccagcaccagagagagcggcgtgcccgacagattcagcggcagcggcagcggcaccgacttcaccctgaccatcagcagcctgcaggccgaggacgtggccgtgtactactgccagaacgcctatagctttccctatacatttggcggtgggaccaagctggagatcaagcggggtgggggcggctctggtggtggaggctccggaggcggtgggagccaggtgcaactacaggagagcggccccggtctgatcaagcccagccagaccctgagcctgacctgcaccgtgagcggcggcagcatcagcagcggctacaactggcactggatcagacagccccccggcaagggcctggagtggatcggctacatccactacaccggcagcaccaactacaaccccgccctgagaagcagagtgaccatcagcgtggacaccagcaagaaccagttcagcctgaagctgagcagcgtgaccgccgccgacaccgccatctactactgcgccagaatctacaacggcaacagcttcccttattggggccagggcaccaccgtgaccgtgagcagc

SEQ ID NO: 143 - protein, 247 aa, synthetic construct
  REGION 1..247: note = standard scFv
DIVMTQSPDSLAVSLGERATINCKSSQSLFNSGNQKNYLTWYQQKPGQPPKLLIYWASTRESGVPDRFSGSGSGTDFTLTISSLQAEDVAVYYCQNAYSFPYTFGGGTKLEIKRGGGGSGGGGSGGGGSQVQLQESGPGLIKPSQTLSLTCTVSGGSISSGYNWHWIRQPPGKGLEWIGYIHYTGSTNYNPALRSRVTISVDTSKNQFSLKLSSVTAADTAIYYCARIYNGNSFPYWGQGTTVTVSS

SEQ ID NO: 144 - protein, 9 aa, synthetic construct
  REGION 1..9: note = 5E6 HCDR3; 5E5 HCDR3; 3A6 HCDR3; 7E3 HCDR3; 14E12 HCDR3; 17B10 HCDR3
  VARIANT 2: note = misc_feature - Xaa = Asn or Tyr
  VARIANT 3: note = misc_feature - Xaa = Arg or Tyr
LXXGNAMDY

SEQ ID NO: 145 - protein, 17 aa, synthetic construct
  REGION 1..17: note = 5E6 HCDR2; 5E5 HCDR2; 3A6 HCDR2; 7E3 HCDR2; 14E12 HCDR2; 17B10 HCDR2
  VARIANT 1: note = misc_feature - Xaa = Thr or Tyr
  VARIANT 3: note = misc_feature - Xaa = Ile or Asn
  VARIANT 4: note = misc_feature - Xaa = Pro or Ser
  VARIANT 5: note = misc_feature - Xaa = Gly or Tyr
  VARIANT 6: note = misc_feature - Xaa = Gly, Asn or Thr
  VARIANT 7: note = misc_feature - Xaa = Asp or Ile
  VARIANT 8: note = misc_feature - Xaa = Gly or Asn
  VARIANT 9: note = misc_feature - Xaa = Pro or Thr
  VARIANT 10: note = misc_feature - Xaa = Lys or Tyr
  VARIANT 12: note = misc_feature - Xaa = Asn or Ser
  VARIANT 13: note = misc_feature - Xaa = Asp or Glu
  VARIANT 14: note = misc_feature - Xaa = Lys, Arg or Ser
  VARIANT 15: note = misc_feature - Xaa = Phe or Val
XIXXXXXXXXYXXXXKG

SEQ ID NO: 146 - skipped

SEQ ID NO: 147 - protein, 118 aa, synthetic construct
  REGION 1..118: note = 5E6 VH; 5E5 VH; 3A6 VH; 7E3 VH; 14E12 VH; 17B10 VH
  VARIANT 1: note = misc_feature - Xaa = Glu or Gln
  VARIANT 5: note = misc_feature - Xaa = Gln or Val
  VARIANT 6: note = misc_feature - Xaa = Glu or Gln
  VARIANT 9: note = misc_feature - Xaa = Ala, Gly or Pro
  VARIANT 10: note = misc_feature - Xaa = Asp or Glu
  VARIANT 11: note = misc_feature - Xaa = Leu or Val
  VARIANT 12: note = misc_feature - Xaa = Lys or Val
  VARIANT 13: note = misc_feature - Xaa = Lys or Gln
  VARIANT 16: note = misc_feature - Xaa = Ala or Gly
  VARIANT 18: note = misc_feature - Xaa = Leu or Val
  VARIANT 20: note = misc_feature - Xaa = Leu, Met or Val
  VARIANT 23: note = misc_feature - Xaa = Ala or Lys
  VARIANT 27: note = misc_feature - Xaa = Phe or Tyr
  VARIANT 29: note = misc_feature - Xaa = Phe or Leu
  VARIANT 30: note = misc_feature - Xaa = Ser or Thr
  VARIANT 31: note = misc_feature - Xaa = Asn or Arg
  VARIANT 33: note = misc_feature - Xaa = Gly, Ile or Val
  VARIANT 34: note = misc_feature - Xaa = Ile or Met
  VARIANT 35: note = misc_feature - Xaa = His, Asn or Ser
  VARIANT 38: note = misc_feature - Xaa = Lys or Arg
  VARIANT 40: note = misc_feature - Xaa = Ala, Lys or Thr
  VARIANT 42: note = misc_feature - Xaa = Asp or Gly
  VARIANT 43: note = misc_feature - Xaa = Lys or Gln
  VARIANT 44: note = misc_feature - Xaa = Gly or Arg
  VARIANT 48: note = misc_feature - Xaa = Ile, Met or Val
  VARIANT 49: note = misc_feature - Xaa = Gly or Ser
  VARIANT 50: note = misc_feature - Xaa = Thr or Tyr
  VARIANT 52: note = misc_feature - Xaa = Ile or Asn
  VARIANT 53: note = misc_feature - Xaa = Pro or Ser
  VARIANT 54: note = misc_feature - Xaa = Gly or Tyr
  VARIANT 55: note = misc_feature - Xaa = Gly, Asn or Thr
  VARIANT 56: note = misc_feature - Xaa = Asp or Ile
  VARIANT 57: note = misc_feature - Xaa = Gly or Asn
  VARIANT 58: note = misc_feature - Xaa = Pro or Thr
  VARIANT 59: note = misc_feature - Xaa = Lys or Tyr
  VARIANT 61: note = misc_feature - Xaa = Asn or Ser
  VARIANT 62: note = misc_feature - Xaa = Asp or Glu
  VARIANT 63: note = misc_feature - Xaa = Lys, Arg or Ser
  VARIANT 64: note = misc_feature - Xaa = Phe or Val
  VARIANT 67: note = misc_feature - Xaa = Lys or Arg
  VARIANT 68: note = misc_feature - Xaa = Ala, Phe or Val
  VARIANT 69: note = misc_feature - Xaa = Ser or Thr
  VARIANT 70: note = misc_feature - Xaa = Ile or Leu
  VARIANT 71: note = misc_feature - Xaa = Ser or Thr
  VARIANT 72: note = misc_feature - Xaa = Arg or Ser
  VARIANT 74: note = misc_feature - Xaa = Lys, Asn or Thr
  VARIANT 75: note = misc_feature - Xaa = Ala or Ser
  VARIANT 76: note = misc_feature - Xaa = Ala, Arg or Ser
  VARIANT 77: note = misc_feature - Xaa = Asn or Ser
  VARIANT 79: note = misc_feature - Xaa = Ala or Leu
  VARIANT 80: note = misc_feature - Xaa = Phe or Tyr
  VARIANT 81: note = misc_feature - Xaa = Leu or Met
  VARIANT 82: note = misc_feature - Xaa = Glu or Gln
  VARIANT 83: note = misc_feature - Xaa = Phe, Leu, Met or Val
  VARIANT 87: note = misc_feature - Xaa = Lys, Arg or Thr
  VARIANT 91: note = misc_feature - Xaa = Ser or Thr
  VARIANT 93: note = misc_feature - Xaa = Met or Val
  VARIANT 97: note = misc_feature - Xaa = Ala or Gly
  VARIANT 100: note = misc_feature - Xaa = Asn or Tyr
  VARIANT 101: note = misc_feature - Xaa = Arg or Tyr
  VARIANT 113: note = misc_feature - Xaa = Ala, Leu or Ser
XVQLXXSGXXXXXPGXSXKXSCXASGXTXXXYXXXWVXQXPXXXLEWXXXIXXXXXXXXYXXXXKGXXXXXXDXXXXTXXXXXSSLXSEDXAXYYCXRLXXGNAMDYWGQGTXVTVSS

SEQ ID NO: 148 - protein, 9 aa, synthetic construct
  REGION 1..9: note = 5E6 LCDR3; 5E5 LCDR3; 7E3 LCDR3; 3A6 LCDR3; 14E12 LCDR3; 17B10 LCDR3
  VARIANT 3: note = misc_feature - Xaa = Asp or Val
  VARIANT 4: note = misc_feature - Xaa = Leu or Tyr
  VARIANT 5: note = misc_feature - Xaa = Ile, Ser or Tyr
  VARIANT 6: note = misc_feature - Xaa = Phe or Tyr
  VARIANT 8: note = misc_feature - Xaa = Phe or Leu
QNXXXXPXT

SEQ ID NO: 149 - protein, 7 aa, synthetic construct
  REGION 1..7: note = 5E6 LCDR2; 5E5 LCDR2; 3A6 LCDR2; 7E3 LCDR2; 17B10 LCDR2; standard LCDR2; 14E12 LCDR2
  VARIANT 1: note = misc_feature - Xaa = Gly or Trp
XASTRES

SEQ ID NO: 150 - protein, 17 aa, synthetic construct
  REGION 1..17: note = 5E6 LCDR1; 5E5 LCDR1; 3A6 LCDR1; 7E3 LCDR1; 17B10 LCDR1; 14E12 LCDR1
  VARIANT 8: note = misc_feature - Xaa = Ala or Asn
  VARIANT 9: note = misc_feature - Xaa = Asn or Ser
  VARIANT 17: note = misc_feature - Xaa = Ala or Thr
KSSQSLLXXGNQKNYLX

SEQ ID NO: 151 - protein, 114 aa, synthetic construct
  REGION 1..114: note = 5E6 VL; 5E5 VL; 7E3 VL; 3A6 VL; 14E12 VL; 17B10 VL
  VARIANT 9: note = misc_feature - Xaa = Asp or Ser
  VARIANT 12: note = misc_feature - Xaa = Ala, Ser or Thr
  VARIANT 14: note = misc_feature - Xaa = Ser or Thr
  VARIANT 15: note = misc_feature - Xaa = Ala, Leu or Val
  VARIANT 16: note = misc_feature - Xaa = Gly or Arg
  VARIANT 18: note = misc_feature - Xaa = Lys or Arg
  VARIANT 19: note = misc_feature - Xaa = Ala or Val
  VARIANT 21: note = misc_feature - Xaa = Ile, Leu or Met
  VARIANT 22: note = misc_feature - Xaa = Asn or Ser
  VARIANT 31: note = misc_feature - Xaa = Ala or Asn
  VARIANT 32: note = misc_feature - Xaa = Asn or Ser
  VARIANT 40: note = misc_feature - Xaa = Ala or Thr
  VARIANT 56: note = misc_feature - Xaa = Gly or Trp
  VARIANT 69: note = misc_feature - Xaa = Ser or Thr
  VARIANT 76: note = misc_feature - Xaa = Asp or His
  VARIANT 78: note = misc_feature - Xaa = Ala or Thr
  VARIANT 84: note = misc_feature - Xaa = Leu or Val
  VARIANT 89: note = misc_feature - Xaa = Leu or Val
  VARIANT 97: note = misc_feature - Xaa = Asp or Val
  VARIANT 98: note = misc_feature - Xaa = Leu or Tyr
  VARIANT 99: note = misc_feature - Xaa = Ile, Ser or Tyr
  VARIANT 100: note = misc_feature - Xaa = Phe or Tyr
  VARIANT 102: note = misc_feature - Xaa = Phe or Leu
  VARIANT 106: note = misc_feature - Xaa = Ala, Gln or Ser
  VARIANT 111: note = misc_feature - Xaa = Asp or Glu
  VARIANT 112: note = misc_feature - Xaa = Ile or Leu
DIVMTQSPXSLXVXXXEXXTXXCKSSQSLLXXGNQKNYLXWYQQKPGQPPKLLIYXASTRESGVPDRFXGSGSGTXFXLTISSXQAEDXAVYYCQNXXXXPXTFGXGTKLXXKR

SEQ ID NO: 152 - protein, 5 aa, synthetic construct
  REGION 1..5: note = MSLN52 HCDR1
YYPKA

SEQ ID NO: 153 - protein, 17 aa, synthetic construct
  REGION 1..17: note = MSLN52 HCDR2
SIGWGGRMTAYADSVKG

SEQ ID NO: 154 - protein, 15 aa, synthetic construct
  REGION 1..15: note = MSLN52 HCDR3
GIGWAPTADSGEYDY

SEQ ID NO: 155 - protein, 124 aa, synthetic construct
  REGION 1..124: note = MSLN52 VHH
EVQLLESGGGLVQPGGSLRLSCAASGHTDSYYPKAWFRQAPGKEREFVASIGWGGRMTAYADSVKGRFTISRDNSKNTLYLQMNSLRAEDTAVYYCAAGIGWAPTADSGEYDYWGQGTLVTVSS

SEQ ID NO: 156 - DNA, 372 bp, synthetic construct
  misc_feature 1..372: note = MSLN52 VHH (DNA)
  source 1..372: mol_type = other DNA
gaggtgcagctcctggaatccggcgggggcctggtgcagcccggcgggagcctcagactgagctgcgctgctagcggccacaccgacagctactaccccaaggcctggttcagacaagcccccggcaaggagagagagttcgtggctagcattggctggggcggcagaatgaccgcctacgccgacagcgtgaagggcagattcaccatcagcagagacaacagcaagaacaccctgtacctgcagatgaacagcctgagagccgaggacacagccgtgtactactgtgccgccggcatcggctgggcccctacagccgacagcggcgagtacgactactggggccaaggcaccctggtgaccgtgagcagc

SEQ ID NO: 157 - protein, 448 aa, synthetic construct
  REGION 1..448: note = zolbetuximab heavy chain
QVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 158 - protein, 220 aa, synthetic construct
  REGION 1..220: note = zolbetuximab light chain
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0177527 A1
(43) Pub. Date: Jun. 5, 2025

(54) CHIMERIC ANTIGEN RECEPTOR TARGETING CLDN18.2 AND MSLN AND USE THEREOF

(71) Applicant: ORICELL THERAPEUTICS CO., LTD., Shanghai (CN)

(72) Inventors: Shasha YANG, Shanghai (CN); Kai WU, Shanghai (CN); Huajing WANG, Shanghai (CN); Huanfeng YANG, Shanghai (CN); Xiaowen HE, Shanghai (CN)

(73) Assignee: ORICELL THERAPEUTICS CO., LTD., Shanghai (CN)

(21) Appl. No.: 18/726,311

(22) PCT Filed: Jan. 6, 2023
(86) PCT No.: PCT/CN2023/070968

§ 371 Date: Jul. 2, 2024

(30) Foreign Application Priority Data
Jan. 7, 2022 (CN) 202210018014.0

(51) Int. Cl. A61K 40/31; A61K 35/17; A61K 40/11; A61K 40/42; A61P 35/00; C07K 16/28; C12N 5/0783

(52) U.S. Cl. CPC A61K 40/31; A61K 35/17; A61K 40/11; A61K 40/4202; A61P 35/00; C07K 16/28; C12N 5/0636; A61K 2239/13; A61K 2239/21; A61K 2239/22; A61K 2239/28; A61K 2239/46; C07K 2317/565; C07K 2317/569; C07K 2317/622; C12N 2510/00

(57) ABSTRACT

Provided is a cell, comprising and/or expressing a chimeric antigen receptor targeting claudin 18.2 (CLDN18.2), and a chimeric antigen receptor targeting mesothelin (MSLN) protein. Also provided is an expression vector, comprising a nucleic acid sequence which encodes the chimeric antigen receptor targeting CLDN18.2 and a nucleic acid sequence which encodes the chimeric antigen receptor targeting MSLN. Also provided are a preparation method for and a use of the cell and/or the expression vector.

### FIELD OF THE INVENTION

The present application relates to the field of biomedicine, and particularly to a chimeric antigen receptor targeting CLDN18.2 and MSLN and use thereof.

### BACKGROUND OF THE INVENTION

Mesothelin (MSLN), a glycoprotein on the cell surface, is anchored on the cell membrane via glycosyl phosphatidyl inositol. The mesothelin gene encodes a precursor protein of 69 kDa, which is hydrolyzed by furin (a paired alkaline amino acid protease)-like converting enzyme into two chains, with a membrane-bound protein of about 40 KD at the C-terminal as the mature mesothelin, and a fragment of about 30 KD at the N-terminal called megakaryocyte-promoting factor (MPF) being shed and released outside the cell. MPF and membrane-anchored MSLN are both N-glycosylated, of which MPF can promote the formation of megakaryocyte clones in vitro, and membrane-anchored MSLN can interact with MUC16 and play an important role in the process of cell adhesion. Therefore, membrane-anchored MSLN is currently selected as the target of targeted therapies, and therefore, at present, MSLN specifically refers to the 40 KD fragment at the C-terminal of MSLN, i.e. membrane-anchored MSLN.

CLDN18.2 (Claudin 18.2) is only expressed on differentiated gastric parietal cells and not expressed in normal tissue. The latest studies show that CLDN18.2 is over-expressed in more than 77% of patients with gastric cancer and more than 80% of patients with pancreatic cancer, as well as in solid tumors such as lung cancer, esophageal cancer and ovarian cancer. CLDN18.2 belongs to a family of tight junction proteins that can control the flow of molecules between layer cells. However, in tumors, tight junctions are disrupted and CLDN proteins lose their primary function. Due to the abundant presence of CLDN18.2 in gastric tumors, researchers estimate that half of all patients with advanced gastric cancer could be candidates for new therapies targeting the CLDN18.2 antibody.

However, existing CAR-T cells targeting CLDN18.2 still face many challenges, primarily in terms of safety and efficacy. Therefore, there is still a need to explore more efficient and safer chimeric antigen receptors to address the issues currently present in CLDN18.2 CAR-T cells.

### SUMMARY OF THE INVENTION

The present application provides a novel chimeric antigen receptor structure and a cell comprising such a chimeric antigen receptor structure. In one aspect, the chimeric antigen receptor and/or the cell provided in the present application can bind to antigens with high affinity. In another aspect, to address the problems of poor safety and limited effectiveness in existing CAR products, the present application combines two or more targets to provide cells capable of expressing chimeric antigen receptors that target different targets. The cell of the present application not only shows significant improvements in safety and efficacy, but also reduces the toxic side effects of CAR-T cells.

In one aspect, the present application provides a cell comprising and/or expressing a chimeric antigen receptor targeting claudin 18.2 (CLDN18.2) and a chimeric antigen receptor targeting mesothelin (MSLN) protein.

In some embodiments, the chimeric antigen receptor targeting CLDN18.2 includes an antigen binding domain.

In some embodiments, the antigen binding domain targeting CLDN18.2 includes at least one CDR in a heavy chain variable region VH, and the VH includes an amino acid sequence as set forth in SEQ ID NO: 147.

In some embodiments, the antigen binding domain targeting CLDN18.2 includes an HCDR3, and the HCDR3 includes an amino acid sequence as set forth in SEQ ID NO: 144.

In some embodiments, the antigen binding domain targeting CLDN18.2 includes an HCDR3, and the HCDR3 includes an amino acid sequence as set forth in any one of SEQ ID NO: 24, SEQ ID NO: 59, and SEQ ID NO: 73.

In some embodiments, the antigen binding domain targeting CLDN18.2 includes an HCDR2, and the HCDR2 includes an amino acid sequence as set forth in SEQ ID NO: 145.

In some embodiments, the antigen binding domain targeting CLDN18.2 includes an HCDR2, and the HCDR2 includes an amino acid sequence as set forth in any one of SEQ ID NO: 22, SEQ ID NO: 57, and SEQ ID NO: 71.

In some embodiments, the antigen binding domain targeting CLDN18.2 includes an HCDR1, and the HCDR1 includes an amino acid sequence as set forth in SEQ ID NO: 146 (X₁YX₂×₃×₄, wherein, X₁ is N or R, X₂ is G, I or V, X₃ is I or M, and X₄ is H, N or S).

In some embodiments, the antigen binding domain targeting CLDN18.2 includes an HCDR1, and the HCDR1 includes an amino acid sequence as set forth in any one of SEQ ID NO: 20, SEQ ID NO: 55, and SEQ ID NO: 69.

In some embodiments, the antigen binding domain targeting CLDN18.2 includes an HCDR1, an HCDR2, and an HCDR3, and the HCDR1, HCDR2, and HCDR3 include any one group of amino acid sequences selected from:
(1) the HCDR1 includes an amino acid sequence as set forth in SEQ ID NO: 20, the HCDR2 includes an amino acid sequence as set forth in SEQ ID NO: 22, and the HCDR3 includes an amino acid sequence as set forth in SEQ ID NO: 24;
(2) the HCDR1 includes an amino acid sequence as set forth in SEQ ID NO: 55, the HCDR2 includes an amino acid sequence as set forth in SEQ ID NO: 57, and the HCDR3 includes an amino acid sequence as set forth in SEQ ID NO: 59; and
(3) the HCDR1 includes an amino acid sequence as set forth in SEQ ID NO: 69, the HCDR2 includes an amino acid sequence as set forth in SEQ ID NO: 71, and the HCDR3 includes an amino acid sequence as set forth in SEQ ID NO: 73.

In some embodiments, the antigen binding domain targeting CLDN18.2 includes a VH, and the VH includes an amino acid sequence as set forth in SEQ ID NO: 147.

In some embodiments, the antigen binding domain targeting CLDN18.2 includes a VH, and the VH includes an amino acid sequence as set forth in any one of SEQ ID NO: 26, SEQ ID NO: 43, SEQ ID NO: 61, SEQ ID NO: 75, and SEQ ID NO: 79.

In some embodiments, the antigen binding domain targeting CLDN18.2 includes at least one CDR in a light chain variable region VL, and the VL includes an amino acid sequence as set forth in SEQ ID NO: 151.

In some embodiments, the antigen binding domain targeting CLDN18.2 includes an LCDR3, and the LCDR3 includes an amino acid sequence as set forth in SEQ ID NO: 148.

In some embodiments, the antigen binding domain targeting CLDN18.2 includes an LCDR3, and the LCDR3 includes an amino acid sequence as set forth in any one of SEQ ID NO: 32, SEQ ID NO: 63. SEQ ID NO: 85, and SEQ ID NO: 91.

In some embodiments, the antigen binding domain targeting CLDN18.2 includes an LCDR2, and the LCDR2 includes an amino acid sequence as set forth in SEQ ID NO: 149.

In some embodiments, the antigen binding domain targeting CLDN18.2 includes an LCDR2, and the LCDR2 includes an amino acid sequence as set forth in SEQ ID NO: 30 or SEQ ID NO: 83.

In some embodiments, the antigen binding domain targeting CLDN18.2 includes an LCDR1, and the LCDR1 includes an amino acid sequence as set forth in SEQ ID NO: 150.

In some embodiments, the antigen binding domain targeting CLDN18.2 includes an LCDR1, and the LCDR1 includes an amino acid sequence as set forth in any one of SEQ ID NO: 28, SEQ ID NO: 45, and SEQ ID NO: 81.

In some embodiments, the antigen binding domain targeting CLDN18.2 includes an LCDR1, an LCDR2, and an LCDR3, and the LCDR1, LCDR2, and LCDR3 include any one group of amino acid sequences selected from:
(1) the LCDR1 includes an amino acid sequence as set forth in SEQ ID NO: 28, the LCDR2 includes an amino acid sequence as set forth in SEQ ID NO: 30, and the LCDR3 includes an amino acid sequence as set forth in SEQ ID NO: 32;
(2) the LCDR1 includes an amino acid sequence as set forth in SEQ ID NO: 45, the LCDR2 includes an amino acid sequence as set forth in SEQ ID NO: 30, and the LCDR3 includes an amino acid sequence as set forth in SEQ ID NO: 32;
(3) the LCDR1 includes an amino acid sequence as set forth in SEQ ID NO: 43, the LCDR2 includes an amino acid sequence as set forth in SEQ ID NO: 30, and the LCDR3 includes an amino acid sequence as set forth in SEQ ID NO: 63;
(4) the LCDR1 includes an amino acid sequence as set forth in SEQ ID NO: 45, the LCDR2 includes an amino acid sequence as set forth in SEQ ID NO: 30, and the LCDR3 includes an amino acid sequence as set forth in SEQ ID NO: 91; and
(5) the LCDR1 includes an amino acid sequence as set forth in SEQ ID NO: 81, the LCDR2 includes an amino acid sequence as set forth in SEQ ID NO: 83, and the LCDR3 includes an amino acid sequence as set forth in SEQ ID NO: 85.

In some embodiments, the antigen binding domain targeting CLDN18.2 includes a VL, and the VL includes an amino acid sequence as set forth in SEQ ID NO: 151.

In some embodiments, the antigen binding domain targeting CLDN18.2 includes a VL, and the VL includes an amino acid sequence as set forth in any one of SEQ ID NO: 34, SEQ ID NO: 49, SEQ ID NO: 65, SEQ ID NO: 87, and SEQ ID NO: 93.

In some embodiments, the antigen binding domain targeting CLDN18.2 includes an HCDR1, an HCDR2, an HCDR3, an LCDR1, an LCDR2, and an LCDR3, and the HCDR1, HCDR2, HCDR3, LCDR1, LCDR2, and LCDR3 include any one group of amino acid sequences selected from:
(1) the HCDR1 includes an amino acid sequence as set forth in SEQ ID NO: 20, the HCDR2 includes an amino acid sequence as set forth in SEQ ID NO: 22, the HCDR3 includes an amino acid sequence as set forth in SEQ ID NO: 24, the LCDR1 includes an amino acid sequence as set forth in SEQ ID NO: 28, the LCDR2 includes an amino acid sequence as set forth in SEQ ID NO: 30, and the LCDR3 includes an amino acid sequence as set forth in SEQ ID NO: 32;
(2) the HCDR1 includes an amino acid sequence as set forth in SEQ ID NO: 20, the HCDR2 includes an amino acid sequence as set forth in SEQ ID NO: 22, the HCDR3 includes an amino acid sequence as set forth in SEQ ID NO: 24, the LCDR1 includes an amino acid sequence as set forth in SEQ ID NO: 45, the LCDR2 includes an amino acid sequence as set forth in SEQ ID NO: 30, and the LCDR3 includes an amino acid sequence as set forth in SEQ ID NO: 32;
(3) the HCDR1 includes an amino acid sequence as set forth in SEQ ID NO: 55, the HCDR2 includes an amino acid sequence as set forth in SEQ ID NO: 57, the HCDR3 includes an amino acid sequence as set forth in SEQ ID NO: 59, the LCDR1 includes an amino acid sequence as set forth in SEQ ID NO: 45, the LCDR2 includes an amino acid sequence as set forth in SEQ ID NO: 30, and the LCDR3 includes an amino acid sequence as set forth in SEQ ID NO: 63;
(4) the HCDR1 includes an amino acid sequence as set forth in SEQ ID NO: 69, the HCDR2 includes an amino acid sequence as set forth in SEQ ID NO: 71, the HCDR3 includes an amino acid sequence as set forth in SEQ ID NO: 73, the LCDR1 includes an amino acid sequence as set forth in SEQ ID NO: 45, the LCDR2 includes an amino acid sequence as set forth in SEQ ID NO: 30, and the LCDR3 includes an amino acid sequence as set forth in SEQ ID NO: 32;
(5) the HCDR1 includes an amino acid sequence as set forth in SEQ ID NO: 69, the HCDR2 includes an amino acid sequence as set forth in SEQ ID NO: 71, the HCDR3 includes an amino acid sequence as set forth in SEQ ID NO: 73, the LCDR1 includes an amino acid sequence as set forth in SEQ ID NO: 45, the LCDR2 includes an amino acid sequence as set forth in SEQ ID NO: 30, and the LCDR3 includes an amino acid sequence as set forth in SEQ ID NO: 91; and
(6) the HCDR1 includes an amino acid sequence as set forth in SEQ ID NO: 69, the HCDR2 includes an amino acid sequence as set forth in SEQ ID NO: 71, the HCDR3 includes an amino acid sequence as set forth in SEQ ID NO: 73, the LCDR1 includes an amino acid sequence as set forth in SEQ ID NO: 81, the LCDR2 includes an amino acid sequence as set forth in SEQ ID NO: 83, and the LCDR3 includes an amino acid sequence as set forth in SEQ ID NO: 85.

In some embodiments, the antigen binding domain targeting CLDN18.2 includes a VH and a VL, and the VH and VL include any one group of amino acid sequences selected from:
(1) the VH includes an amino acid sequence as set forth in SEQ ID NO: 26, and the VL includes an amino acid sequence as set forth in SEQ ID NO: 34;
(2) the VH includes an amino acid sequence as set forth in SEQ ID NO: 43, and the VL includes an amino acid sequence as set forth in SEQ ID NO: 49;
(3) the VH includes an amino acid sequence as set forth in SEQ ID NO: 75, and the VL includes an amino acid sequence as set forth in SEQ ID NO: 49;
(4) the VH includes an amino acid sequence as set forth in SEQ ID NO: 61, and the VL includes an amino acid sequence as set forth in SEQ ID NO: 65;
(5) the VH includes an amino acid sequence as set forth in SEQ ID NO: 79, and the VL includes an amino acid sequence as set forth in SEQ ID NO: 93; and
(6) the VH includes an amino acid sequence as set forth in SEQ ID NO: 79, and the VL includes an amino acid sequence as set forth in SEQ ID NO: 87.

In some embodiments, the antigen binding domain targeting CLDN18.2 includes an scFv.

In some embodiments, the scFv targeting CLDN18.2 includes an amino acid sequence as set forth in any one of SEQ ID NO: 38, SEQ ID NO: 53, SEQ ID NO: 67, SEQ ID NO: 77, SEQ ID NO: 89, and SEQ ID NO: 95.

In some embodiments, the chimeric antigen receptor targeting CLDN18.2 includes a costimulatory domain, and the costimulatory domain includes a costimulatory domain derived from one or more proteins selected from a group consisting of: CD28, 4-1BB, CD27, CD2, CD7, CD8, OX40, CD226, DR3, SLAM, CDS, ICAM-1, NKG2D, NKG2C, B7-H3, 2B4, FcεRIγ, BTLA, GITR, HVEM, DAP10, DAP12, CD30, CD40, CD40L, TIM1, PD-1, LFA-1, LIGHT, JAML, CD244, CD100, ICOS, a ligand of CD83, CD40, and MyD88.

In some embodiments, the costimulatory domain is an intracellular costimulatory signaling domain derived from 4-1BB.

In some embodiments, the costimulatory domain includes an amino acid sequence as set forth in SEQ ID NO: 8.

In some embodiments, the chimeric antigen receptor targeting CLDN18.2 includes a transmembrane region, and the transmembrane region includes a transmembrane region derived from one or more proteins selected from a group consisting of: CD8, CD28, 4-1BB, CD4, CD27, CD7, PD-1, TRAC, TRBC, CD3ε, CD3ζ, CTLA-4, LAG-3, CD5, ICOS, OX40, NKG2D, 2B4, CD244, FcεRIγ, BTLA, CD30, GITR, HVEM, DAP10, CD2, NKG2C, LIGHT, DAP12, CD40L, TIM1, CD226, DR3, CD45, CD80, CD86, CD9, CD16, CD22, CD33, CD37, CD64, CD134, CD137, CD154, and SLAM.

In some embodiments, the transmembrane region is a transmembrane region derived from CD8.

In some embodiments, the transmembrane region is a transmembrane region derived from human CD8.

In some embodiments, the transmembrane region includes an amino acid sequence as set forth in SEQ ID NO: 6.

In some embodiments, the chimeric antigen receptor targeting CLDN18.2 includes a hinge region, and the hinge region includes a hinge region derived from one or more proteins selected from a group consisting of: CD28, IgG1, IgG4, IgD, 4-1BB, CD4, CD27, CD7, CD8, PD-1, ICOS, OX40, NKG2D, NKG2C, FcεRIγ, BTLA, GITR, DAP10, CD40L, TIM1, CD226, SLAM, CD30, and LIGHT.

In some embodiments, the hinge region is a hinge region derived from CD8.

In some embodiments, the hinge region is a hinge region derived from human CD8.

In some embodiments, the hinge region includes an amino acid sequence as set forth in SEQ ID NO: 4.

In some embodiments, the chimeric antigen receptor targeting CLDN18.2 includes a signal peptide.

In some embodiments, the signal peptide is derived from a signal peptide of CD8 protein.

In some embodiments, the signal peptide includes an amino acid sequence as set forth in SEQ ID NO: 2.

In some embodiments, the chimeric antigen receptor targeting CLDN18.2 does not include an intracellular signaling domain.

In some embodiments, the chimeric antigen receptor targeting MSLN includes an antigen binding domain.

In some embodiments, the antigen binding domain of the chimeric antigen receptor targeting MSLN includes at least one CDR in a heavy chain variable region VH, and the VH includes an amino acid sequence as set forth in any one of SEQ ID NO: 103, SEQ ID NO: 107, SEQ ID NO: 111, SEQ ID NO: 120, and SEQ ID NO: 155.

In some embodiments, the antigen binding domain of the chimeric antigen receptor targeting MSLN includes an HCDR3, and the HCDR3 includes an amino acid sequence as set forth in any one of SEQ ID NO: 101, SEQ ID NO: 106, SEQ ID NO: 110, SEQ ID NO: 119, and SEQ ID NO: 154.

In some embodiments, the antigen binding domain of the chimeric antigen receptor targeting MSLN includes an HCDR2, and the HCDR2 includes an amino acid sequence as set forth in any one of SEQ ID NO: 99, SEQ ID NO: 105, SEQ ID NO: 109, SEQ ID NO: 118, and SEQ ID NO: 153.

In some embodiments, the antigen binding domain of the chimeric antigen receptor targeting MSLN includes an HCDR1, and the HCDR1 includes an amino acid sequence as set forth in any one of SEQ ID NO: 97. SEQ ID NO: 104. SEQ ID NO: 108. SEQ ID NO: 117, and SEQ ID NO: 152.

In some embodiments, the antigen binding domain of the chimeric antigen receptor targeting MSLN includes an HCDR1, an HCDR2, and an HCDR3, and the HCDR1, HCDR2, and HCDR3 includes any one group of amino acid sequences selected from:
(1) the HCDR1 includes an amino acid sequence as set forth in SEQ ID NO: 97, the HCDR2 includes an amino acid sequence as set forth in SEQ ID NO: 99, and the HCDR3 includes an amino acid sequence as set forth in SEQ ID NO: 101;
(2) the HCDR1 includes an amino acid sequence as set forth in SEQ ID NO: 104, the HCDR2 includes an amino acid sequence as set forth in SEQ ID NO: 105, and the HCDR3 includes an amino acid sequence as set forth in SEQ ID NO: 106;
(3) the HCDR1 includes an amino acid sequence as set forth in SEQ ID NO: 108, the HCDR2 includes an amino acid sequence as set forth in SEQ ID NO: 109, and the HCDR3 includes an amino acid sequence as set forth in SEQ ID NO: 110;
(4) the HCDR1 includes an amino acid sequence as set forth in SEQ ID NO: 117, the HCDR2 includes an amino acid sequence as set forth in SEQ ID NO: 118, and the HCDR3 includes an amino acid sequence as set forth in SEQ ID NO: 119; and
(5) the HCDR1 includes an amino acid sequence as set forth in SEQ ID NO: 152, the HCDR2 includes an amino acid sequence as set forth in SEQ ID NO: 153, and the HCDR3 includes an amino acid sequence as set forth in SEQ ID NO: 154.

In some embodiments, the antigen binding domain of the chimeric antigen receptor targeting MSLN includes a VH, and the VH includes an amino acid sequence as set forth in any one of SEQ ID NO: 103, SEQ ID NO: 107, SEQ ID NO: 111, SEQ ID NO: 120, and SEQ ID NO: 155.

In some embodiments, the antigen binding domain of the chimeric antigen receptor targeting MSLN includes a VHH.

The cell of claim 49, wherein the VHH includes an amino acid sequence as set forth in any one of SEQ ID NO: 103, SEQ ID NO: 107, and SEQ ID NO: 155.

In some embodiments, the antigen binding domain of the chimeric antigen receptor targeting MSLN includes an LCDR3, and the LCDR3 includes an amino acid sequence as set forth in SEQ ID NO: 123 or SEQ ID NO: 114.

In some embodiments, the antigen binding domain of the chimeric antigen receptor targeting MSLN includes an LCDR2, and the LCDR2 includes an amino acid sequence as set forth in SEQ ID NO: 113 or SEQ ID NO: 122.

In some embodiments, the antigen binding domain of the chimeric antigen receptor targeting MSLN includes an LCDR1, and the LCDR1 includes an amino acid sequence as set forth in SEQ ID NO: 112 or SEQ ID NO: 121.

In some embodiments, the antigen binding domain of the chimeric antigen receptor targeting MSLN includes an LCDR1, an LCDR2, and an LCDR3, and the LCDR1, LCDR2, and LCDR3 includes any one group of amino acid sequences selected from:
(1) the LCDR1 includes an amino acid sequence as set forth in SEQ ID NO: 112, the LCDR2 includes an amino acid sequence as set forth in SEQ ID NO: 113, and the LCDR3 includes an amino acid sequence as set forth in SEQ ID NO: 114; and
(2) the LCDR1 includes an amino acid sequence as set forth in SEQ ID NO: 121, the LCDR2 includes an amino acid sequence as set forth in SEQ ID NO: 122, and the LCDR3 includes an amino acid sequence as set forth in SEQ ID NO: 123.

In some embodiments, the antigen binding domain of the chimeric antigen receptor targeting MSLN includes a VL, and the VL includes an amino acid sequence as set forth in SEQ ID NO: 115 or SEQ ID NO: 124.

In some embodiments, the antigen binding domain of the chimeric antigen receptor targeting MSLN includes an scFv, and the scFv includes an amino acid sequence as set forth in SEQ ID NO: 116 or SEQ ID NO: 125.

In some embodiments, the chimeric antigen receptor targeting MSLN includes an intracellular signaling domain, and the intracellular signaling domain includes an intracellular signaling domain derived from one or more proteins selected from a group consisting of: CD3ζ, CD3δ, CD3γ, CD3ε, CD79a, CD79b, FcεRIγ, FcεRIβ, FcγRIIa, bovine leukemia virus gp30, Epstein-Barr virus (EBV) LMP2A, simian immunodeficiency virus PBj14 Nef, Kaposi's sarcoma-associated herpesvirus (HSKV), DAP10, DAP-12, and a domain containing at least one ITAM.

In some embodiments, the intracellular signaling domain is a signaling domain derived from CD3ζ.

In some embodiments, the intracellular signaling domain includes an amino acid sequence as set forth in SEQ ID NO: 16.

In some embodiments, the chimeric antigen receptor targeting MSLN includes a transmembrane region, and the transmembrane region includes a transmembrane region derived from one or more proteins selected from a group consisting of: CD8, CD28, 4-1BB, CD4, CD27, CD7, PD-1, TRAC, TRBC, CD3ε, CD3ζ, CTLA-4, LAG-3, CD5, ICOS, OX40, NKG2D, 2B4, CD244, FcεRIγ, BTLA, CD30, GITR, HVEM, DAP10, CD2, NKG2C, LIGHT, DAP12, CD40L, TIM1, CD226, DR3, CD45, CD80, CD86, CD9, CD16, CD22, CD33, CD37, CD64, CD134, CD137, CD154, and SLAM.

In some embodiments, the transmembrane region is a transmembrane region derived from CD28.

In some embodiments, the transmembrane region includes an amino acid sequence as set forth in SEQ ID NO: 14.

In some embodiments, the chimeric antigen receptor targeting MSLN includes a hinge region, and the hinge region includes a hinge region derived from one or more proteins selected from a group consisting of: CD28, IgG1, IgG4, IgD, 4-1BB, CD4, CD27, CD7, CD8, PD-1, ICOS, OX40, NKG2D, NKG2C, FcεRIγ, BTLA, GITR, DAP10, CD40L, TIM1, CD226, SLAM, CD30, and LIGHT.

In some embodiments, the hinge region is a hinge region derived from CD28.

In some embodiments, the hinge region includes an amino acid sequence as set forth in SEQ ID NO: 12.

In some embodiments, the chimeric antigen receptor targeting MSLN includes a signal peptide.

In some embodiments, the signal peptide is derived from a signal peptide of CD8 protein.

In some embodiments, the signal peptide includes an amino acid sequence as set forth in SEQ ID NO: 2.

In some embodiments, the chimeric antigen receptor targeting MSLN further includes a low-density lipoprotein receptor-related protein or a fragment thereof.

In some embodiments, the low-density lipoprotein receptor-related protein or the fragment thereof includes one or more selected from a group consisting of: low-density lipoprotein receptor-related proteins 1-12 and functional fragments thereof.

In some embodiments, the low-density lipoprotein receptor-related protein or the fragment thereof is low-density lipoprotein receptor-related proteins 5 and/or 6 or fragments thereof.

In some embodiments, the low-density lipoprotein receptor-related protein or the fragment thereof includes an amino acid sequence as set forth in SEQ ID NO: 18.

In some embodiments, the chimeric antigen receptor targeting MSLN does not include a costimulatory domain.

In some embodiments, in the cell, the expression level of the chimeric antigen receptor targeting CLDN18.2 is approximately 1:1 with that of the chimeric antigen receptor targeting MSLN.

In some embodiments, in the cell, the expression level of the chimeric antigen receptor targeting CLDN18.2 is approximately 2:1 with that of the chimeric antigen receptor targeting MSLN.

In some embodiments, the cell further includes and/or expresses a low-density lipoprotein receptor-related protein or a fragment thereof.

In some embodiments, the low-density lipoprotein receptor-related protein or the fragment thereof includes one or more selected from a group consisting of: low-density lipoprotein receptor-related proteins 1-12 and functional fragments thereof.

In some embodiments, the low-density lipoprotein receptor-related protein or the fragment thereof is low-density lipoprotein receptor-related proteins 5 and/or 6 or fragments thereof.

In some embodiments, the low-density lipoprotein receptor-related protein or the fragment thereof includes an amino acid sequence as set forth in SEQ ID NO: 18.

In some embodiments, the cell includes an immune effector cell.

In some embodiments, the cell includes T cells, B cells, natural killer cells (NK cells), macrophages, NKT cells, monocytes, dendritic cells, granulocytes, lymphocytes, leukocytes, peripheral blood mononuclear cells, embryonic stem cells, lymphoid progenitor cells and/or pluripotent stem cells.

In some embodiments, the cell is a T cell.

In another aspect, the present application provides an expression vector, which includes a nucleic acid sequence which encodes the chimeric antigen receptor targeting CLDN18.2 and a nucleic acid sequence which encodes the chimeric antigen receptor targeting MSLN.

In another aspect, the present application provides an expression vector, which includes a nucleic acid sequence which encodes the chimeric antigen receptor targeting CLDN18.2 of the present application and a nucleic acid sequence which encodes the chimeric antigen receptor targeting MSLN of the present application.

In some embodiments, in the expression vector, the nucleic acid sequence which encodes the chimeric antigen receptor targeting CLDN18.2 includes a nucleic acid sequence which encodes the antigen binding domain.

In some embodiments, the antigen binding domain targeting CLDN18.2 includes at least one CDR in a VH, and a nucleic acid sequence which encodes the VH includes a nucleic acid sequence as set forth in any one of SEQ ID NO: 25, SEQ ID NO: 42, SEQ ID NO: 60, SEQ ID NO: 74, and SEQ ID NO: 78.

In some embodiments, the antigen binding domain targeting CLDN18.2 includes an HCDR3, and a nucleic acid sequence which encodes the HCDR3 includes a nucleic acid sequence as set forth in any one of SEQ ID NO: 23, SEQ ID NO: 41, SEQ ID NO: 58, and SEQ ID NO: 72.

In some embodiments, the antigen binding domain targeting CLDN18.2 includes an HCDR2, and a nucleic acid sequence which encodes the HCDR2 includes a nucleic acid sequence as set forth in any one of SEQ ID NO: 21, SEQ ID NO: 40, SEQ ID NO: 56, and SEQ ID NO: 70.

In some embodiments, the antigen binding domain targeting CLDN18.2 includes an HCDR1, and a nucleic acid sequence which encodes the HCDR1 includes a nucleic acid sequence as set forth in any one of SEQ ID NO: 19, SEQ ID NO: 39, SEQ ID NO: 54, and SEQ ID NO: 68.

In some embodiments, the antigen binding domain targeting CLDN18.2 includes a VH, and a nucleic acid sequence which encodes the VH includes a nucleic acid sequence as set forth in any one of SEQ ID NO: 25, SEQ ID NO: 42, SEQ ID NO: 60, SEQ ID NO: 74, and SEQ ID NO: 78.

In some embodiments, the antigen binding domain targeting CLDN18.2 includes an LCDR3, and a nucleic acid sequence which encodes the LCDR3 includes a nucleic acid sequence as set forth in any one of SEQ ID NO: 31, SEQ ID NO: 47, SEQ ID NO: 62, SEQ ID NO: 84, and SEQ ID NO: 90.

In some embodiments, the antigen binding domain targeting CLDN18.2 includes an LCDR2, and a nucleic acid sequence which encodes the LCDR2 includes a nucleic acid sequence as set forth in any one of SEQ ID NO: 29, SEQ ID NO: 46, and SEQ ID NO: 82.

In some embodiments, the antigen binding domain targeting CLDN18.2 includes an LCDR1, and a nucleic acid sequence which encodes the LCDR1 includes a nucleic acid sequence as set forth in any one of SEQ ID NO: 27, SEQ ID NO: 44, and SEQ ID NO: 80.

In some embodiments, the antigen binding domain targeting CLDN18.2 includes a VL, and a nucleic acid sequence which encodes the VL includes a nucleic acid sequence as set forth in any one of SEQ ID NO: 33, SEQ ID NO: 48, SEQ ID NO: 64, SEQ ID NO: 86, and SEQ ID NO: 92.

In some embodiments, the antigen binding domain targeting CLDN18.2 includes an scFv.

In some embodiments, a nucleic acid sequence which encodes the scFv includes a nucleic acid sequence as set forth in any one of SEQ ID NO: 37, SEQ ID NO: 52, SEQ ID NO: 66, SEQ ID NO: 76, SEQ ID NO: 88, and SEQ ID NO: 94.

In some embodiments, the nucleic acid sequence which encodes the chimeric antigen receptor targeting CLDN18.2 includes a nucleic acid sequence which encodes a costimulatory domain, and the costimulatory domain includes is a costimulatory domain derived from one or more proteins selected from a group consisting of: CD28, 4-1BB, CD27, CD2, CD7, CD8, OX40, CD226, DR3, SLAM, CDS, ICAM-1, NKG2D, NKG2C, B7-H3, 2B4, FcεRIγ, BTLA, GITR, HVEM, DAP10, DAP12, CD30, CD40, CD40L, TIM1, PD-1, LFA-1, LIGHT, JAML, CD244, CD100, ICOS, a ligand of CD83, CD40, and MyD88.

In some embodiments, the costimulatory domain is an intracellular costimulatory signaling domain derived from 4-1BB.

In some embodiments, the nucleic acid sequence which encodes the costimulatory domain includes a nucleic acid sequence as set forth in SEQ ID NO: 7.

In some embodiments, the nucleic acid sequence which encodes the chimeric antigen receptor targeting CLDN18.2 includes a nucleic acid sequence which encodes a transmembrane region, and the transmembrane region includes a transmembrane region derived from one or more proteins selected from a group consisting of: CD8, CD28, 4-1BB, CD4, CD27, CD7, PD-1, TRAC, TRBC, CD3ε, CD3ζ, CTLA-4, LAG-3, CD5, ICOS, OX40, NKG2D, 2B4, CD244, FcεRIγ, BTLA, CD30, GITR, HVEM, DAP10, CD2, NKG2C, LIGHT. DAP12, CD40L, TIM1, CD226, DR3, CD45, CD80, CD86, CD9, CD16, CD22, CD33, CD37, CD64, CD134, CD137, CD154, and SLAM.

In some embodiments, the transmembrane region is a transmembrane region derived from CD8.

In some embodiments, the nucleic acid sequence which encodes the transmembrane region includes a nucleic acid sequence as set forth in SEQ ID NO: 5.

In some embodiments, the nucleic acid sequence which encodes the chimeric antigen receptor targeting CLDN18.2 includes a nucleic acid sequence which encodes a hinge region, and the hinge region includes a hinge region derived from one or more proteins selected from a group consisting of: CD28, IgG1, IgG4, IgD, 4-1BB, CD4, CD27, CD7, CD8, PD-1, ICOS, OX40, NKG2D, NKG2C, FcεRIγ, BTLA, GITR, DAP10, CD40L, TIM1, CD226, SLAM, CD30, and LIGHT.

In some embodiments, the hinge region is a hinge region derived from CD8.

In some embodiments, the nucleic acid sequence which encodes the hinge region includes a nucleic acid sequence as set forth in SEQ ID NO: 5.

In some embodiments, the nucleic acid sequence which encodes the chimeric antigen receptor targeting CLDN18.2 includes a nucleic acid sequence which encodes a signal peptide.

In some embodiments, the signal peptide is derived from a signal peptide of CD8 protein.

In some embodiments, the nucleic acid sequence which encodes the signal peptide includes a nucleic acid sequence as set forth in SEQ ID NO: 1.

In some embodiments, the nucleic acid sequence which encodes the chimeric antigen receptor targeting CLDN18.2 does not include a nucleic acid sequence which encodes an intracellular signaling domain.

In some embodiments, the nucleic acid sequence which encodes the chimeric antigen receptor targeting MSLN includes a nucleic acid sequence which encodes an antigen binding domain.

In some embodiments, the antigen binding domain targeting MSLN includes at least one CDR in a heavy chain variable region VH, and the VH includes an amino acid sequence as set forth in any one of SEQ ID NO: 103, SEQ ID NO: 107, SEQ ID NO: 111, SEQ ID NO: 120, and SEQ ID NO: 155.

In some embodiments, the nucleic acid sequence which encodes the VH includes a nucleic acid sequence as set forth in SEQ ID NO: 102 or SEQ ID NO: 156.

In some embodiments, the antigen binding domain targeting MSLN includes an HCDR3, and the HCDR3 includes an amino acid sequence as set forth in any one of SEQ ID NO: 101, SEQ ID NO: 106, SEQ ID NO: 110, SEQ ID NO: 119, and SEQ ID NO: 154. In some embodiments, the nucleic acid sequence which encodes the HCDR3 includes a nucleic acid sequence as set forth in SEQ ID NO: 100.

In some embodiments, the antigen binding domain targeting MSLN includes an HCDR2, and the HCDR2 includes an amino acid sequence as set forth in any one of SEQ ID NO: 99, SEQ ID NO: 105, SEQ ID NO: 109, SEQ ID NO: 118, and SEQ ID NO: 153. In some embodiments, the nucleic acid sequence which encodes the HCDR2 includes a nucleic acid sequence as set forth in SEQ ID NO: 98.

In some embodiments, the antigen binding domain targeting MSLN includes an HCDR1, and the HCDR1 includes an amino acid sequence as set forth in any one of SEQ ID NO: 97, SEQ ID NO: 104, SEQ ID NO: 108, SEQ ID NO: 117, and SEQ ID NO: 152. In some embodiments, the nucleic acid sequence which encodes the HCDR1 includes a nucleic acid sequence as set forth in SEQ ID NO: 96.

In some embodiments, the antigen binding domain targeting MSLN includes a VH, and the VH includes an amino acid sequence as set forth in any one of SEQ ID NO: 103, SEQ ID NO: 107, SEQ ID NO: 111, SEQ ID NO: 120, and SEQ ID NO: 155. In some embodiments, the nucleic acid sequence which encodes the VH includes a nucleic acid sequence as set forth in SEQ ID NO: 102 or SEQ ID NO: 156.

In some embodiments, the antigen binding domain targeting MSLN includes a VHH, and the VHH includes an amino acid sequence as set forth in SEQ ID NO: 103, SEQ ID NO: 107, and SEQ ID NO: 155. In some embodiments, the nucleic acid sequence which encodes the VHH includes a nucleic acid sequence as set forth in SEQ ID NO: 102 or SEQ ID NO: 156.

In some embodiments, the antigen binding domain targeting MSLN includes an LCDR3, and the LCDR3 includes an amino acid sequence as set forth in any one of SEQ ID NO: 114 and SEQ ID NO: 123. In some embodiments, the nucleic acid sequence which encodes the LCDR3 includes a nucleic acid sequence as set forth in SEQ ID NO: 123 or SEQ ID NO: 114.

In some embodiments, the antigen binding domain targeting MSLN includes an LCDR2, and the LCDR2 includes an amino acid sequence as set forth in SEQ ID NO: 113 or SEQ ID NO: 122.

In some embodiments, the antigen binding domain targeting MSLN includes an LCDR1, and the LCDR1 includes an amino acid sequence as set forth in SEQ ID NO: 112 or SEQ ID NO: 121.

In some embodiments, the antigen binding domain targeting MSLN includes a VL, and the VL includes an amino acid sequence as set forth in SEQ ID NO: 115 or SEQ ID NO: 124.

In some embodiments, the antigen binding domain targeting MSLN includes an scFv, and the scFv includes an amino acid sequence as set forth in SEQ ID NO: 116 or SEQ ID NO: 125.

In some embodiments, the nucleic acid sequence which encodes the chimeric antigen receptor targeting MSLN includes a nucleic acid sequence which encodes an intracellular signaling domain, and the intracellular signaling domain includes an intracellular signaling domain derived from one or more proteins selected from a group consisting of: CD3ζ, CD3δ, CD3γ, CD3ε, CD79a, CD79b, FcεRIγ, FcεRIβ, FcγRIIa, bovine leukemia virus gp30, Epstein-Barr virus (EBV) LMP2A, simian immunodeficiency virus PBj14 Nef, Kaposi's sarcoma-associated herpesvirus (HSKV), DAP10, DAP-12, and a domain containing at least one ITAM.

In some embodiments, the intracellular signaling domain is a signaling domain derived from CD3ζ.

In some embodiments, the nucleic acid sequence which encodes the intracellular signaling domain includes a nucleic acid sequence as set forth in SEQ ID NO: 15.

In some embodiments, the nucleic acid sequence which encodes the chimeric antigen receptor targeting MSLN includes a nucleic acid sequence which encodes a transmembrane region, and the transmembrane region includes a transmembrane region derived from one or more proteins selected from a group consisting of: CD8, CD28, 4-1BB, CD4, CD27, CD7, PD-1, TRAC. TRBC, CD3ε, CD3ζ, CTLA-4, LAG-3, CD5, ICOS, OX40, NKG2D, 2B4, CD244, FcεRIγ, BTLA, CD30, GITR, HVEM, DAP10, CD2, NKG2C, LIGHT, DAP12, CD40L, TIM1, CD226, DR3, CD45, CD80, CD86, CD9, CD16, CD22, CD33, CD37, CD64, CD134, CD137, CD154, and SLAM.

In some embodiments, the transmembrane region is a transmembrane region derived from CD28.

In some embodiments, the nucleic acid sequence which encodes the transmembrane region includes a nucleic acid sequence as set forth in SEQ ID NO: 13.

In some embodiments, the nucleic acid sequence which encodes the chimeric antigen receptor targeting MSLN includes a nucleic acid sequence which encodes a hinge region, and the hinge region includes a hinge region derived from one or more proteins selected from a group consisting of: CD28, IgG1, IgG4, IgD, 4-1BB, CD4, CD27, CD7, CD8, PD-1, ICOS, OX40, NKG2D, NKG2C, FcεRIγ, BTLA, GITR, DAP10, CD40L, TIM1, CD226, SLAM, CD30, and LIGHT.

In some embodiments, the hinge region is a hinge region derived from CD28.

In some embodiments, the nucleic acid sequence which encodes the hinge region includes a nucleic acid sequence as set forth in SEQ ID NO: 11.

In some embodiments, the nucleic acid sequence which encodes the chimeric antigen receptor targeting CLDN18.2 includes a nucleic acid sequence which encodes a signal peptide.

In some embodiments, the signal peptide is derived from a signal peptide of CD8 protein.

In some embodiments, the nucleic acid sequence which encodes the signal peptide includes a nucleic acid sequence as set forth in SEQ ID NO: 1.

In some embodiments, the nucleic acid sequence which encodes the chimeric antigen receptor targeting MSLN does not include a nucleic acid sequence which encodes the costimulatory domain.

In some embodiments, the nucleic acid sequence which encodes the chimeric antigen receptor targeting CLDN18.2 is linked to the nucleic acid sequence which encodes the chimeric antigen receptor targeting MSLN via a sequence which encodes a cleaving peptide.

In some embodiments, the cleaving peptide is selected from a group consisting of: P2A, T2A, F2A, and E2A.

In some embodiments, the cleaving peptide includes an amino acid sequence as set forth in SEQ ID NO: 10.

In some embodiments, the nucleic acid sequence which encodes the cleaving peptide includes a nucleic acid sequence as set forth in SEQ ID NO: 9.

In some embodiments, the expression vector includes at least one nucleic acid sequence which encodes the chimeric antigen receptor targeting CLDN18.2.

In some embodiments, the expression vector includes two nucleic acid sequences which encode the chimeric antigen receptor targeting CLDN18.2.

In some embodiments, the two nucleic acid sequences which encode the chimeric antigen receptor targeting CLDN18.2 are linked to each other via a nucleic acid sequence which encodes a cleaving peptide.

In some embodiments, the cleaving peptide is selected from a group consisting of: P2A, T2A, F2A, and E2A.

In some embodiments, in the expression vector, the nucleic acid sequence which encodes the cleaving peptide includes a nucleic acid sequence as set forth in SEQ ID NO: 9.

In some embodiments, the expression vector further includes a nucleic acid sequence which encodes a low-density lipoprotein receptor-related protein or a fragment thereof.

In some embodiments, the low-density lipoprotein receptor-related protein or the fragment thereof includes one or more selected from a group consisting of: low-density lipoprotein receptor-related proteins 1-12 and functional fragments thereof.

In some embodiments, the low-density lipoprotein receptor-related protein or the fragment thereof is low-density lipoprotein receptor-related proteins 5 and/or 6 or fragments thereof.

In some embodiments, the nucleic acid sequence which encodes the low-density lipoprotein receptor-related protein or the fragment thereof includes a nucleic acid sequence as set forth in SEQ ID NO: 17.

In some embodiments, the nucleic acid sequence which encodes the low-density lipoprotein receptor-related protein or the fragment thereof is linked to the nucleic acid sequence which encodes the intracellular signaling domain of the chimeric antigen receptor targeting MSLN directly.

In some embodiments, the nucleic acid sequence which encodes the low-density lipoprotein receptor-related protein or the fragment thereof is linked to the nucleic acid sequence which encodes the chimeric antigen receptor targeting MSLN via a cleaving peptide.

In some embodiments, the nucleic acid sequence which encodes the chimeric antigen receptor targeting CLDN18.2 and the nucleic acid sequence which encodes the chimeric antigen receptor targeting MSLN are located in the same nucleic acid molecule.

In some embodiments, the nucleic acid sequence which encodes the chimeric antigen receptor targeting CLDN18.2, the nucleic acid sequence which encodes the chimeric antigen receptor targeting MSLN, and the nucleic acid sequence which encodes the low-density lipoprotein receptor-related protein or the fragment thereof are located in the same nucleic acid molecule.

In some embodiments, in the expression vector, a first nucleic acid sequence which encodes the chimeric antigen receptor targeting CLDN18.2, a second nucleic acid sequence which encodes the chimeric antigen receptor targeting CLDN18.2, a nucleic acid sequence which encodes the chimeric antigen receptor targeting MSLN, and a nucleic acid sequence which encodes the low-density lipoprotein receptor-related protein or the fragment thereof are located in the same nucleic acid molecule.

In some embodiments, the expression vector includes, sequentially from 5′ end to 3′ end, a nucleic acid sequence which encodes the chimeric antigen receptor targeting CLDN18.2, a nucleic acid sequence which encodes the cleaving peptide, a nucleic acid sequence which encodes the chimeric antigen receptor targeting MSLN, a nucleic acid sequence which encodes the low-density lipoprotein receptor-related protein or the fragment thereof.

In some embodiments, the expression vector includes, sequentially from 5′ end to 3′ end, a nucleic acid sequence which encodes the chimeric antigen receptor targeting CLDN18.2, a nucleic acid sequence which encodes the cleaving peptide, a nucleic acid sequence which encodes the chimeric antigen receptor targeting CLDN18.2, a nucleic acid sequence which encodes the cleaving peptide, a nucleic acid sequence which encodes the chimeric antigen receptor targeting MSLN, a nucleic acid sequence which encodes the low-density lipoprotein receptor-related protein or the fragment thereof.

In some embodiments, the expression vector includes, sequentially from 5′ end to 3′ end, a nucleic acid sequence which encodes the signal peptide, a nucleic acid sequence which encodes CLDN18.2 scFv, a nucleic acid sequence which encodes the hinge region, a nucleic acid sequence which encodes the transmembrane region, a nucleic acid sequence which encodes the costimulatory domain, a nucleic acid sequence which encodes the cleaving peptide, a nucleic acid sequence which encodes the signal peptide, a nucleic acid sequence which encodes MSLN VHH, a nucleic acid sequence which encodes the hinge region, a nucleic acid sequence which encodes the transmembrane region, a nucleic acid sequence which encodes the intracellular signaling domain, and a nucleic acid sequence which encodes the low-density lipoprotein receptor-related protein or the fragment thereof.

In some embodiments, the expression vector includes, sequentially from 5′ end to 3′ end, a nucleic acid sequence which encodes the CD8 signal peptide, a nucleic acid sequence which encodes CLDN18.2 scFv, a nucleic acid sequence which encodes the CD8 hinge region, a nucleic acid sequence which encodes the CD8 transmembrane region, a nucleic acid sequence which encodes the 4-1BB costimulatory domain, a nucleic acid sequence which encodes the cleaving peptide, a nucleic acid sequence which encodes the CD8 signal peptide, a nucleic acid sequence which encodes MSLN VHH, a nucleic acid sequence which encodes the CD28 hinge region, a nucleic acid sequence which encodes the CD28 transmembrane region, a nucleic acid sequence which encodes the CD3ζ intracellular signaling domain, and a nucleic acid sequence which encodes the low-density lipoprotein receptor-related protein or the fragment thereof.

In some embodiments, the expression vector includes, sequentially from 5′ end to 3′ end, a nucleic acid sequence which encodes the signal peptide, a nucleic acid sequence which encodes CLDN18.2 scFv, a nucleic acid sequence which encodes the hinge region, a nucleic acid sequence which encodes the transmembrane region, a nucleic acid sequence which encodes the costimulatory domain, a nucleic acid sequence which encodes the cleaving peptide, a nucleic acid sequence which encodes the signal peptide, a nucleic acid sequence which encodes CLDN18.2 scFv, a nucleic acid sequence which encodes the hinge region, a nucleic acid sequence which encodes the transmembrane region, a nucleic acid sequence which encodes the costimulatory domain, a nucleic acid sequence which encodes the cleaving peptide, a nucleic acid sequence which encodes the signal peptide, a nucleic acid sequence which encodes MSLN VHH, a nucleic acid sequence which encodes the hinge region, a nucleic acid sequence which encodes the transmembrane region, a nucleic acid sequence which encodes the intracellular signaling domain, and a nucleic acid sequence which encodes the low-density lipoprotein receptor-related protein or the fragment thereof.

In some embodiments, the expression vector includes, sequentially from 5′ end to 3′ end, a nucleic acid sequence which encodes the CD8 signal peptide, a nucleic acid sequence which encodes CLDN18.2 scFv, a nucleic acid sequence which encodes the CD8 hinge region, a nucleic acid sequence which encodes the CD8 transmembrane region, a nucleic acid sequence which encodes the 4-1BB costimulatory domain, a nucleic acid sequence which encodes the cleaving peptide, a nucleic acid sequence which encodes the CD8 signal peptide, a nucleic acid sequence which encodes CLDN18.2 scFv, a nucleic acid sequence which encodes the CD8 hinge region, a nucleic acid sequence which encodes the CD8 transmembrane region, a nucleic acid sequence which encodes the 4-1BB costimulatory domain, a nucleic acid sequence which encodes the cleaving peptide, a nucleic acid sequence which encodes the CD8 signal peptide, a nucleic acid sequence which encodes MSLN VHH, a nucleic acid sequence which encodes the CD28 hinge region, a nucleic acid sequence which encodes the CD28 transmembrane region, a nucleic acid sequence which encodes the CD3ζ intracellular signaling domain, and a nucleic acid sequence which encodes the low-density lipoprotein receptor-related protein or the fragment thereof.

In another aspect, the present application further provides a cell, which includes the expression vector of the present application.

In some embodiments, the cell is an immune effector cell.

In some embodiments, the cell includes T cells, B cells, natural killer cells (NK cells), macrophages, NKT cells, monocytes, dendritic cells, granulocytes, lymphocytes, leukocytes, peripheral blood mononuclear cells, embryonic stem cells, lymphoid progenitor cells and/or pluripotent stem cells.

In some embodiments, the cell is a T cell.

In another aspect, the present application further provides use of the cell and/or the expression vector in the preparation of a drug for preventing and/or treating a disease and/or a disorder.

In another aspect, the present application further provides use of the chimeric antigen receptor targeting CLDN18.2 and the chimeric antigen receptor targeting MSLN in the preparation of a drug for preventing and/or treating a disease and/or a disorder.

In some embodiments, the disease and/or disorder includes a tumor.

In some embodiments, the tumor includes a solid tumor and/or a non-solid tumor.

In some embodiments, the tumor includes a tumor simultaneously expressing both antigens CLDN18.2 and MSLN.

In some embodiments, the tumor includes gastric cancer, pancreatic cancer, and/or gastroesophageal junction carcinoma.

In another aspect, the present application further provides a method for preventing and/or treating a disease and/or a disorder, which includes administering to a subject in need thereof the cell of the present application.

In some embodiments, the disease and/or disorder includes a tumor.

In some embodiments, the tumor includes a solid tumor and/or a non-solid tumor.

In some embodiments, the tumor includes a tumor simultaneously expressing both antigens CLDN18.2 and MSLN.

In some embodiments, the tumor includes gastric cancer, pancreatic cancer, and/or gastroesophageal junction carcinoma.

Those skilled in the art can easily perceive other aspects and advantages of the present application from the detailed description below. In the following detailed description, only exemplary embodiments of the present application are shown and described. As those skilled in the art will recognize, the content of the present application enables those skilled in the art to make changes to the disclosed specific embodiments without departing from the spirit and scope of the invention involved in the present application. Correspondingly, the drawings and descriptions in the specification of the present application are merely exemplary, rather than restrictive.

### BRIEF DESCRIPTION OF THE DRAWINGS

The specific features of the invention involved in the present application are shown in the appended claims. The characteristics and advantages of the invention involved in the present application can be better understood by referring to the exemplary embodiments and the accompanying drawings described in detail below. A brief description of the drawings is as follows:

FIG. 1 shows the antigen binding activity of a CLDN18.2 single-chain antibody.

FIG. 2 shows the identification on the binding activity of a CLDN18.2 single-chain antibody to non-target cells.

FIGS. 3A-3D show the stable expression of CLDN18.2-specific CARs in T cells.

FIG. 4 shows the experimental results of CLDN18.2-specific CARs specifically killing target cells in vitro.

FIGS. 5A-5B show the factor secretion of CLDN18.2-specific CARs.

FIGS. 6A-6D show the antitumor effect of CLDN18.2-specific CAR-T cells in mouse colon cancer CDX models.

FIGS. 7A-7B show schematic diagrams of the construction of the dual CARs described in the present application.

FIGS. 8A-8B show the expression and proliferation of the CAR-T cells described in the present application.

FIG. 9 shows the in vim killing activity of CAR-T cells.

FIGS. 10A-10B show the secretion of cytokines by the CAR-T cells described in the present application.

FIGS. 11A-II C show the antitumor effect of the CAR-T cells described in the present application in human gastric cancer CDX models.

FIGS. 12A-12E show the toxicological experiment of the CAR-T cells described in the present application in mice.

FIG. 13A shows a schematic diagram of the construction of the CARs described in the present application; FIGS. 13B-13C show the expression of the CARs described in the present application in cells and the proliferation of the CAR-T cells.

FIG. 14 shows the proliferation of the CAR-T cells described in the present application under repeated stimulation of target cells.

FIG. 15 shows the in vitro cell killing profile of the CAR-T cells described in the present application.

FIG. 16 shows the detection of the in vitro cytokine secretion of the CAR-T cells described in the present application.

FIGS. 17A-17D show the antitumor effect of the CAR-T cells described in the present application in human gastric cancer mouse CDX models.

FIG. 18 shows the detection of the binding activity of the CLDN18.2 targeting moiety described in the present application to cells highly expressing human CLDN18.2 by flow cytometry.

FIG. 19 shows the detection of the binding activity of the CLDN18.2 targeting moiety described in the present application to cells highly expressing human CLDN18.1 by flow cytometry.

FIG. 20 shows the detection of the binding activity of the CLDN18.2 targeting moiety described in the present application to tumor cell lines by flow cytometry.

### DETAILED DESCRIPTION OF THE EMBODIMENTS

The implementation of the present application will be illustrated in the following specific examples, and other advantages and effects of the present application will be easily known by those skilled in the art from the content disclosed in the specification.

### Definition of Terms

In the present application, the term “CLDN18.2” or “Claudin18.2” can be used interchangeably and generally refers to a subtype 2 of Claudin18. The term encompasses “full-length” unprocessed CLDN18.2 as well as any forms of CLDN18.2 produced by cell processing. CLDN18.2 may include intact CLDN18.2 and fragments, functional variants, isoforms, species homologs, derivatives, and analogs thereof, as well as analogs having at least one common epitope with CLDN18.2. The amino acid sequence CLDN18.2 (e.g., human CLDN18.2) may be known in the art. For example, the nucleotide sequence of human CLDN18.2 may be shown under GeneBank Accession No. NM_001002026.3. For example, the nucleotide sequence of mouse CLDN18.2 may be shown under GeneBank Accession No. NM_001194921.1. For example, the nucleotide sequence of Cynomolgus monkey CLDN18.2 may be shown under GeneBank Accession No. XM_001114708.4.

In the present application, the terms “variable domain” and “variable region” may be used interchangeably and generally refers to a part of an antibody heavy chain and/or light chain. The heavy chain and light chain variable domains may be referred to “VH” and “VL” (or referred to “VH” and “VL”), respectively. These domains are generally the most variable parts of the antibody (relative to other antibodies of the same type) and include antigen binding sites.

In the present application, the term “variable” generally refers to a fact that there may be a great difference in the sequences of some segments of the variable domains among antibodies. The variable domain mediates the binding of antigens and determines the specificity of a specific antibody to its specific antigens. However, variability is not evenly distributed throughout the variable domain. Instead, it is generally concentrated in three segments called hypervariable regions (CDRs or HVRs) in the light chain and heavy chain variable domains. The more highly conserved part of the variable domain is referred to a framework region (FR). The variable domains of natural heavy chains and light chains each includes four FR regions, most of which are in β-folded configuration in which they are connected by three CDRs to form a circular connection and in some cases form a part of a β-folded structure. The CDRs in each chain arc held together closely by the FR region and promote the formation of the antigen binding site of the antibody together with the CDRs from another chain (see Kabat et al, Sequences of Immunological Interest, Fifth Edition, National Institute of Health, Bethesda, Md. (1991)).

In the present application, the term “CDR”, also known as “complementary determining region”, generally refers to a region in an antibody variable domain, of which the sequence is highly variable and/or forms a structure-defining ring. Generally, an antibody includes six CDRs; three in the VH (HCDR1, HCDR2, HCDR3) and three in the VL (LCDR1, LCDR2, LCDR3). In some embodiments, naturally occurring camel antibodies only composed of heavy chains function normally and are stable in the absence of light chains. See, for example, Hamers-Casterman et al., Nature 363:446-448 (1993); Sheriff et al, Nature Struct. Biol. 3:733-736 (1996).

In the present application, the term “antigen binding fragment” generally refers to one or more fragments having the ability of specifically binding to an antigen (e.g., CLDN18.2). In the present application, the antigen binding fragment may include Fab, Fab′, F(ab)₂, an Fv fragment, F(ab′)₂, scFv, di-scFv, VHH, and/or dAb.

In the present application, the term “Fab” generally refers to an antigen binding fragment of an antibody. As mentioned above, intact antibodies can be digested using papain. The antibody is digested with papain to produce two identical antigen binding fragments, i.e., the “Fab” fragment and the residual “Fc” fragment (i.e. Fc region, ibid.). The Fab fragment may consist of a complete L chain, a variable region of a heavy chain and a first constant region (CHI) of the H chain (VH).

In the present application, the term “Fab′ fragment” generally refers to a monovalent antigen binding fragment of a human monoclonal antibody that is slightly larger than a Fab fragment. For example, the Fab′ fragment may include all light chains, all heavy chain variable regions, and all or part of the first and second constant regions of the heavy chain. For example, the Fab′ fragment may also include some or all of the 220-330 amino acid residues of the heavy chain.

In the present application, the term “F(ab′)2” generally refers to an antibody fragment produced by digesting an intact antibody with pepsin. The F(ab′)2 fragment contains two Fab fragments held together by a disulfide bond and part of the hinge region. The F(ab′)2 fragment have bivalent antigen binding activity and is capable of cross-linking antigens.

In the present application, the term “Fv fragment” generally refers to a monovalent antigen binding fragment of a human monoclonal antibody, including all or part of the heavy chain variable region and the light chain variable region, and lacking the heavy chain constant region and the light chain constant region. Heavy chain variable regions and light chain variable regions include, e.g., CDRs. For example, the Fv fragment includes all or part of the amino-terminal variable regions of about 110 amino acids of the heavy chain and light chain.

In the present application, the term “scFv” generally refers to a fusion protein comprising at least one antibody fragment comprising a variable region of a light chain and at least one antibody fragment comprising a variable region of a heavy chain, wherein the light chain and heavy chain variable regions are contiguous (e.g., via a synthetic linker, e.g., a short flexible polypeptide linker) and capable of being expressed in the form of a single-chain polypeptide, and wherein the scFv retains the specificity of the intact antibody from which it is derived. Unless otherwise specified, as used in the present application, the scFv may have the VL and VH variable regions described in any order (e.g., relative to the N-terminus and C-terminus of the polypeptide), and the scFv may include VL-linker-VH or may include VH-linker-VL.

In the present application, the term “dAb” generally refers to an antigen binding fragment having a VH domain and a VL domain, or a VH domain or a VL domain, with reference to, e.g., Ward et al. (Nature, 1989 Oct. 12; 341 (6242): 544-6), Holt et al., Trends Biotechnol., 2003, 21 (11): 484-490; and for example, WO 06/030220, WO 06/003388, and other published patent applications to Domantis Ltd.

In the present application, the term “linked directly” is opposite to the term “linked indirectly”. The term “linked directly” generally refers to direct linking. For example, the direct linking may be a case that the substances are directly linked without spacers. The spacers may be linkers. For example, the linkers may be peptide linkers. The term “indirect linking” generally refers to a case that the substances are not directly linked. For example, the indirect linking may be linking through spacers.

In the present application, the term “isolated nucleic acid molecules” generally refer to isolated nucleotides, deoxyribonucleoides or ribonucleotides of any length, or analogs thereof isolated from its natural environment or synthesized artificially.

In the present application, the term “vector” generally refers to a nucleic acid delivery vehicle into which a polynucleotide encoding a protein can be inserted so as to enable the expression of the protein. The vector can make the genetic elements it carries be expressed in a host cell by transforming, transducing or transfecting the host cell. For example, the vector may include plasmid; phagemid; Cosmid; artificial chromosomes, such as yeast artificial chromosomes (YAC), bacterial artificial chromosomes (BAC) or P1-derived artificial chromosomes (PAC); phages, such as lambda phages or M13 phages and animal viruses, and the like. The species of animal viruses used as the vector may include retrovirus (including lentivirus), adenovirus, adeno-associated virus, herpes virus (e.g., herpes simplex virus), poxvirus, baculovirus, papilloma virus, papovavirus (e.g., SV40). A vector may contain various elements for controlling the expression, including promoter sequences, transcription initiation sequences, enhancer sequences, selective elements and reporter genes. In addition, the vector may also contain replication initiation sites. The vector may also probably include ingredients that help its entry into cells, such as virion, lipidosome or protein coat, but not only these substances.

In the present application, the term “cell” generally refers to a single cell, cell line, or cell culture that can be or has been a recipient of a subject's plasmid or vector, which includes the nucleic acid molecules of the present application or the vector of the present application. The cell may include the offsprings of a single cell. Due to natural, accidental or intentional mutations, the offsprings may not necessarily be exactly the same as the original parent cells (in the form of the total DNA complement or in the genome). The cell may include cells transfected with the vector of the present application in vitro. The cell may be bacterial cells (e.g., E. coli), yeast cells, or other eukaryotic cells, such as COS cells, Chinese Hamster Ovary (CHO) cells, CHO-K1 cells, LNCAP cells, HeLa cells, HEK293 cells, COS-1 cells, NSO cells or stem cells (e.g., ES cells, iPS cells, mesenchymal stem cells) or immune cells (e.g., T cells, NK cells, NKT cells, macrophages).

In the present application, the term “pharmaceutical composition” generally refers to a composition for preventing/treating a disease or disorder. The pharmaceutical composition may include the chimeric antigen receptor of the present application, the nucleic acid molecule of the present application, the vector of the present application and/or the cell of the present application, as well as optionally a pharmaceutically acceptable adjuvant. In addition, the pharmaceutical composition may further include one or more (pharmaceutically effective) carriers, stabilizers, excipients, diluents, solubilizers, surfactants, emulsifiers and/or preservatives and other suitable preparations. The acceptable ingredients of the composition are non-toxic to the recipient at the dosage and concentration used. The pharmaceutical composition of the present application includes, but not limited to, liquid, frozen and lyophilized compositions.

In the present application, the term “pharmaceutically acceptable carrier” generally includes pharmaceutically acceptable carriers, excipients or stabilizers that are non-toxic to cells or mammals to which they are exposed at the dosage and concentration used. Physiologically acceptable carriers may include, e.g., buffers, antioxidants, low molecular weight (less than about 10 residues) polypeptides, proteins, hydrophilic polymers, amino acids, monosaccharide, disaccharide, and other carbohydrates, chelating agents, sugar alcohols, salt-forming counter ions, e.g. sodium, and/or nonionic surfactants.

In the present application, the term “specifically binding to” or “specific” generally refers to measurable and reproducible interactions, such as the binding between targets and antibodies, which can determine the presence of the targets in the presence of a heterogeneous population of molecules, including biomolecules. For example, an antibody specifically binding to a target (which may be an epitope) is an antibody that binds to the target with greater affinity, avidity, easier, and/or for a greater duration than it binds to other targets. In some embodiments, the antibody specifically binds to the epitope on the protein, and the epitope is conservative among proteins of different species. In some embodiments, specifical binding may include, but does not require exclusive binding.

In the present application, the term “subject” generally refers to human or non-human animals, including but not limited to cat, dog, horse, pig, cow, sheep, rabbit, mouse, rat, or monkey.

In the present application, the term “tumor” generally refers to neoplasm or solid lesion formed by the growth of abnormal cells. In the present application, the tumor may be a solid tumor or a blood tumor. For example, in the present application, the tumor may be a CLDN18.2-positive tumor.

The term “cancer” generally refers to diseases characterized by the rapid and uncontrolled growth of abnormal cells. Cancer cells can spread to other parts of the body, either locally or through the bloodstream and the lymphatic system. Cancers in the present application include, but not limited to, gastric cancer and/or colon cancer. The terms “tumor” and “cancer” can be used interchangably. For example, the two terms encompass solid tumors and liquid tumors, e.g., diffuse or circulating tumors. As used herein, the term “cancer” or “tumor” may include premalignant and malignant cancers and tumors.

The protein, polypeptide and/or amino acid sequences involved in the present application should also be understood to include at least the following ranges: variants or homologs having the same or similar functions as those of the protein or polypeptide.

In the present application, the variants may be, e.g., proteins or polypeptides with one or more amino acid substitutions, deletions or additions in the amino acid sequence of the protein and/or the polypeptide (e.g., an antibody or a fragment thereof specifically binding to CLDN18.2 protein). For example, the functional variants may include proteins or polypeptides with amino acid changes by at least 1, for example, 1-30, 1-20 or 1-10, and further for example, 1, 2, 3, 4 or S amino acid substitutions, deletions and/or insertions. The functional variants can substantially maintain the biological properties of the protein or the polypeptide before change (e.g., substitution, deletion or addition). For example, the functional variants can maintain at least 60%, 70%, 80%, 90%, or 100% of the biological activity (e.g., antigen binding ability) of the protein or the polypeptide before change. For example, the substitution may be conservative substitution.

In the present application, the homologs may be proteins or polypeptides having at least about 85% (e.g., at least about 85%, about 90%, about 91%, about 92%, about 93%, about 94%, about 95%, about 96%, about 97%, about 98%, about 99% or higher) sequence homology with the amino acid sequence of the protein, and/or the polypeptide (e.g., an antibody or a fragment thereof specifically binding to CLDN18.2 protein).

In the present application, the homology generally refers to the similarity, likeness or correlation between two or more sequences. The “percentage of sequence homology” can be calculated by: comparing two sequences to be aligned in a comparison window to determine the number of positions where the same nucleic acid bases (e.g., A, T, C, G, I) or the same amino acid residues (e.g., Ala, Pro, Ser, Thr, Gly, Val, Leu, Ile, Phe, Tyr, Trp, Lys, Arg, His, Asp, Glu, Asn, Gln, Cys, and Met) are present in both sequences so as to obtain the number of matching positions, dividing the number of matching positions by the total number of positions in the comparison window (i.e., window size), and multiplying the result by 100, to generate the percentage of sequence homology. The alignment for determining the percentage of sequence homology can be achieved in a variety of ways known in the art, for example, by using publicly available computer softwares, such as BLAST, BLAST-2, ALIGN or Megalign (DNASTAR) softwares. A person skilled in the an can determine appropriate parameters for aligning sequences, including any algorithms needed to achieve the maximum alignment over the full-length sequence range being compared or within the target sequence region. The homology can also be determined by the following methods: FASTA and BLAST. A description of FASTA algorithm can be found in “Improved tools for biological sequence comparison” to W. R. Pearson and D. J. Lipman, Proc. Natl. Acad. Sci., 85: 2444-2448, 1988; and “Rapid and Sensitive Protein Similarity Searches” to D. J. Lipman and W. R. Pearson, Science, 227: 1435-1441, 1989. A description of BLAST algorithm can be found in “Basic Local Alignment Search Tool” to S. Altschul, W. Gish, W. Miller, E. W. Myers and D. Lipman, Journal of Molecular Biology, 215: 403-410, 1990.

In the present application, the term “include” generally refers to the meaning of include, encompass, contain or embrace. In some cases, it also indicates the meaning of “is”, or “composed of . . . ”.

In the present application, the term “about” generally refers to varying in a range of 0.5%-10% above or below a specified value, for example, varying in a range of 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5% or 10% above or below a specified value.

### DETAILED DESCRIPTION OF THE INVENTION

In one aspect, the present application provides a cell, which includes and/or expresses a chimeric antigen receptor targeting claudin 18.2 (CLDN18.2) and a chimeric antigen receptor targeting mesothelin (MSLN) protein.

In another aspect, the present application provides an expression vector, which includes a nucleic acid sequence which encodes the chimeric antigen receptor targeting CLDN18.2, and a nucleic acid sequence which encodes the chimeric antigen receptor targeting MSLN.

In another aspect, the present application further provides a cell, which includes the expression vector of the present application.

### Chimeric Antigen Receptor Targeting CLDN18.2

In the present application, the chimeric antigen receptor targeting CLDN18.2 includes an antigen binding domain targeting CLDN18.2.

In some embodiments, the antigen binding domain targeting CLDN18.2 includes at least one CDR in a heavy chain variable region VH, and the VH includes an amino acid sequence as set forth in SEQ ID NO: 147.

In some embodiments, the antigen binding domain targeting CLDN18.2 includes an HCDR3, and the HCDR3 includes an amino acid sequence as set forth in SEQ ID NO: 144.

In some embodiments, the antigen binding domain targeting CLDN18.2 includes an HCDR3, and the HCDR3 includes an amino acid sequence as set forth in any one of SEQ ID NO: 24, SEQ ID NO: 39, and SEQ ID NO: 73.

In some embodiments, the antigen binding domain targeting CLDN18.2 includes an HCDR2, and the HCDR2 includes an amino acid sequence as set forth in SEQ ID NO: 145.

In some embodiments, the antigen binding domain targeting CLDN18.2 includes an HCDR2, and the HCDR2 includes an amino acid sequence as set forth in any one of SEQ ID NO: 22, SEQ ID NO: 57, and SEQ ID NO: 71.

In some embodiments, the antigen binding domain targeting CLDN18.2 includes an HCDR1, and the HCDR1 includes an amino acid sequence as set forth in SEQ ID NO: 146 (X₁YX₂×₃×₄, wherein X₁ is N or R, X₂ is G, I or V, X₃ is I or M, and X₄ is H, N or S).

In some embodiments, the antigen binding domain targeting CLDN18.2 includes an HCDR1, and the HCDR1 includes an amino acid sequence as set forth in any one of SEQ ID NO: 20, SEQ ID NO: 55, and SEQ ID NO: 69.

In some embodiments, the antigen binding domain targeting CLDN18.2 includes an HCDR1, an HCDR2, and an HCDR3, and the HCDR1, HCDR2, and HCDR3 includes any one group of amino acid sequences selected from:
(1) the HCDR1 includes an amino acid sequence as set forth in SEQ ID NO: 20, the HCDR2 includes an amino acid sequence as set forth in SEQ ID NO: 22, and the HCDR3 includes an amino acid sequence as set forth in SEQ ID NO: 24;
(2) the HCDR1 includes an amino acid sequence as set forth in SEQ ID NO: 55, the HCDR2 includes an amino acid sequence as set forth in SEQ ID NO: 57, and the HCDR3 includes an amino acid sequence as set forth in SEQ ID NO: 59; and
(3) the HCDR1 includes an amino acid sequence as set forth in SEQ ID NO: 69, the HCDR2 includes an amino acid sequence as set forth in SEQ ID NO: 71, and the HCDR3 includes an amino acid sequence as set forth in SEQ ID NO: 73.

In some embodiments, the antigen binding domain targeting CLDN18.2 includes a VH, and the VH includes an amino acid sequence as set forth in SEQ ID NO: 147.

In some embodiments, the antigen binding domain targeting CLDN18.2 includes a VH, and the VH includes an amino acid sequence as set forth in any one of SEQ ID NO: 26, SEQ ID NO: 43, SEQ ID NO: 61, SEQ ID NO: 75, and SEQ ID NO: 79.

In some embodiments, the antigen binding domain targeting CLDN18.2 includes at least one CDR in a light chain variable region VL, and the VL includes an amino acid sequence as set forth in SEQ ID NO: 151.

In some embodiments, the antigen binding domain targeting CLDN18.2 includes an LCDR3, and the LCDR3 includes an amino acid sequence as set forth in SEQ ID NO: 148.

In some embodiments, the antigen binding domain targeting CLDN182 includes an LCDR3, and the LCDR3 includes an amino acid sequence as set forth in any one of SEQ ID NO: 32, SEQ ID NO: 63, SEQ ID NO: 85, and SEQ ID NO: 91.

In some embodiments, the antigen binding domain targeting CLDN182 includes an LCDR2, and the LCDR2 includes an amino acid sequence as set forth in SEQ ID NO: 149.

In some embodiments, the antigen binding domain targeting CLDN18.2 includes an LCDR2, and the LCDR2 includes an amino acid sequence as set forth in any one of SEQ ID NO: 30 and SEQ ID NO: 83.

In some embodiments, the antigen binding domain targeting CLDN18.2 includes an LCDR1, and the LCDR1 includes an amino acid sequence as set forth in SEQ ID NO: 150.

In some embodiments, the antigen binding domain targeting CLDN18.2 includes an LCDR1, and the LCDR1 includes an amino acid sequence as set forth in any one of SEQ ID NO: 28, SEQ ID NO: 45, and SEQ ID NO: 81.

In some embodiments, the antigen binding domain targeting CLDN18.2 includes an LCDR1, an LCDR2, and an LCDR3, and the LCDR1, LCDR2, and LCDR3 includes any one group of amino acid sequences selected from:
(1) the LCDR1 includes an amino acid sequence as set forth in SEQ ID NO: 28, the LCDR2 includes an amino acid sequence as set forth in SEQ ID NO: 30, and the LCDR3 includes an amino acid sequence as set forth in SEQ ID NO: 32;
(2) the LCDR1 includes an amino acid sequence as set forth in SEQ ID NO: 45, the LCDR2 includes an amino acid sequence as set forth in SEQ ID NO: 30, and the LCDR3 includes an amino acid sequence as set forth in SEQ ID NO: 32;
(3) the LCDR1 includes an amino acid sequence as set forth in SEQ ID NO: 45, the LCDR2 includes an amino acid sequence as set forth in SEQ ID NO: 30, and the LCDR3 includes an amino acid sequence as set forth in SEQ ID NO: 63;
(4) the LCDR1 includes an amino acid sequence as set forth in SEQ ID NO: 45, the LCDR2 includes an amino acid sequence as set forth in SEQ ID NO: 30, and the LCDR3 includes an amino acid sequence as set forth in SEQ ID NO: 91; and
(5) the LCDR1 includes an amino acid sequence as set forth in SEQ ID NO: 81, the LCDR2 includes an amino acid sequence as set forth in SEQ ID NO: 83, and the LCDR3 includes an amino acid sequence as set forth in SEQ ID NO: 85.

In some embodiments, the antigen binding domain targeting CLDN18.2 includes a VL, and the VL includes an amino acid sequence as set forth in SEQ ID NO: 151.

In some embodiments, the antigen binding domain targeting CLDN18.2 includes a VL, and the VL includes an amino acid sequence as set forth in any one of SEQ ID NO: 34, SEQ ID NO: 49, SEQ ID NO: 65, SEQ ID NO: 87, and SEQ ID NO: 93.

In some embodiments, the antigen binding domain targeting CLDN18.2 includes an HCDR1, an HCDR2, an HCDR3, an LCDR1, an LCDR2, and an LCDR3, and the HCDR1, HCDR2, HCDR3, LCDR1, LCDR2, and LCDR3 includes any one group of amino acid sequences selected from:
(1) the HCDR1 includes an amino acid sequence as set forth in SEQ ID NO: 20, the HCDR2 includes an amino acid sequence as set forth in SEQ ID NO: 22, the HCDR3 includes an amino acid sequence as set forth in SEQ ID NO: 24, the LCDR1 includes an amino acid sequence as set forth in SEQ ID NO: 28, the LCDR2 includes an amino acid sequence as set forth in SEQ ID NO: 30, and the LCDR3 includes an amino acid sequence as set forth in SEQ ID NO: 32;
(2) the HCDR1 includes an amino acid sequence as set forth in SEQ ID NO: 20, the HCDR2 includes an amino acid sequence as set forth in SEQ ID NO: 22, the HCDR3 includes an amino acid sequence as set forth in SEQ ID NO: 24, the LCDR1 includes an amino acid sequence as set forth in SEQ ID NO: 45, the LCDR2 includes an amino acid sequence as set forth in SEQ ID NO: 30, and the LCDR3 includes an amino acid sequence as set forth in SEQ ID NO: 32;
(3) the HCDR1 includes an amino acid sequence as set forth in SEQ ID NO: 55, the HCDR2 includes an amino acid sequence as set forth in SEQ ID NO: 57, the HCDR3 includes an amino acid sequence as set forth in SEQ ID NO: 59, the LCDR1 includes an amino acid sequence as set forth in SEQ ID NO: 45, the LCDR2 includes an amino acid sequence as set forth in SEQ ID NO: 30, and the LCDR3 includes an amino acid sequence as set forth in SEQ ID NO: 63;
(4) the HCDR1 includes an amino acid sequence as set forth in SEQ ID NO: 69, the HCDR2 includes an amino acid sequence as set forth in SEQ ID NO: 71, the HCDR3 includes an amino acid sequence as set forth in SEQ ID NO: 73, the LCDR1 includes an amino acid sequence as set forth in SEQ ID NO: 45, the LCDR2 includes an amino acid sequence as set forth in SEQ ID NO: 30, and the LCDR3 includes an amino acid sequence as set forth in SEQ ID NO: 32;
(5) the HCDR1 includes an amino acid sequence as set forth in SEQ ID NO: 69, the HCDR2 includes an amino acid sequence as set forth in SEQ ID NO: 71, the HCDR3 includes an amino acid sequence as set forth in SEQ ID NO: 73, the LCDR1 includes an amino acid sequence as set forth in SEQ ID NO: 45, the LCDR2 includes an amino acid sequence as set forth in SEQ ID NO: 30, and the LCDR3 includes an amino acid sequence as set forth in SEQ ID NO: 91; and
(6) the HCDR1 includes an amino acid sequence as set forth in SEQ ID NO: 69, the HCDR2 includes an amino acid sequence as set forth in SEQ ID NO: 71, the HCDR3 includes an amino acid sequence as set forth in SEQ ID NO: 73, the LCDR1 includes an amino acid sequence as set forth in SEQ ID NO: 81, the LCDR2 includes an amino acid sequence as set forth in SEQ ID NO: 83, and the LCDR3 includes an amino acid sequence as set forth in SEQ ID NO: 85.

In some embodiments, the antigen binding domain targeting CLDN18.2 includes a VH and a VL, and the VH and VL include any one group of amino acid sequences selected from:
(1) the VH includes an amino acid sequence as set forth in SEQ ID NO: 26, and the VL includes an amino acid sequence as set forth in SEQ ID NO: 34;
(2) the VH includes an amino acid sequence as set forth in SEQ ID NO: 43, and the VL includes an amino acid sequence as set forth in SEQ ID NO: 49;
(3) the VH includes an amino acid sequence as set forth in SEQ ID NO: 75, and the VL includes an amino acid sequence as set forth in SEQ ID NO: 49;
(4) the VH includes an amino acid sequence as set forth in SEQ ID NO: 61, and the VL includes an amino acid sequence as set forth in SEQ ID NO: 63;
(5) the VH includes an amino acid sequence as set forth in SEQ ID NO: 79, and the VL includes an amino acid sequence as set forth in SEQ ID NO: 93; and
(6) the VH includes an amino acid sequence as set forth in SEQ ID NO: 79, and the VL includes an amino acid sequence as set forth in SEQ ID NO: 87.

In some embodiments, the antigen binding domain targeting CLDN18.2 includes an scFv.

In some embodiments, the scFv targeting CLDN18.2 includes an amino acid sequence as set forth in any one of SEQ ID NO: 38, SEQ ID NO: 53, SEQ ID NO: 67, SEQ ID NO: 77, SEQ ID NO: 89, and SEQ ID NO: 95.

In some embodiments, the chimeric antigen receptor targeting CLDN18.2 includes a costimulatory domain, and the costimulatory domain includes a costimulatory domain derived from one or more proteins selected from a group consisting of: CD28, 4-1BB, CD27, CD2, CD7, CD8, OX40, CD226, DR3, SLAM, CDS, ICAM-1, NKG2D, NKG2C, B7-H3, 2B4, FcεRIγ, BTLA, GITR, HVEM, DAP10, DAP12, CD30, CD40, CD40L, TIM1, PD-1, LFA-1, LIGHT, JAML, CD244, CD100, ICOS, a ligand of CD83, CD40, and MyD88.

In some embodiments, the costimulatory domain is an intracellular costimulatory signaling domain derived from 4-1BB.

In some embodiments, the costimulatory domain includes an amino acid sequence as set forth in SEQ ID NO: 8.

In some embodiments, the chimeric antigen receptor targeting CLDN18.2 includes a transmembrane region, and the transmembrane region includes a transmembrane region derived from one or more proteins selected from a group consisting of: CD8, CD28, 4-1BB, CD4, CD27, CD7, PD-1, TRAC, TRBC, CD3ε, CD3ζ, CTLA-4, LAG-3, CD5, ICOS, OX40, NKG2D, 2B4, CD244, FcεRIγ, BTLA, CD30, GITR, HVEM, DAP10, CD2, NKG2C, LIGHT, DAP12, CD40L, TIM1, CD226, DR3, CD45, CD80, CD86, CD9, CD16, CD22, CD33, CD37, CD64, CD134, CD137, CD154, and SLAM.

In some embodiments, the transmembrane region is a transmembrane region derived from CD8.

In some embodiments, the transmembrane region is a transmembrane region derived from human CD8.

In some embodiments, the transmembrane region includes an amino acid sequence as set forth in SEQ ID NO: 6.

In some embodiments, the chimeric antigen receptor targeting CLDN18.2 includes a hinge region, and the hinge region includes a hinge region derived from one or more proteins selected from a group consisting of: CD28, IgG1, IgG4, IgD, 4-1BB, CD4, CD27, CD7, CD8, PD-1, ICOS, OX40, NKG2D, NKG2C, FcεRIγ, BTLA, GITR, DAP10, CD40L, TIM1, CD226, SLAM, CD30, and LIGHT.

In some embodiments, the hinge region is a hinge region derived from CD8.

In some embodiments, the hinge region is a hinge region derived from human CD8.

In some embodiments, the hinge region includes an amino acid sequence as set forth in SEQ ID NO: 4.

In some embodiments, the chimeric antigen receptor targeting CLDN18.2 includes a signal peptide.

In some embodiments, the signal peptide is derived from a signal peptide of CD8 protein.

In some embodiments, the signal peptide includes an amino acid sequence as set forth in SEQ ID NO: 2.

In some embodiments, the chimeric antigen receptor targeting CLDN18.2 does not include an intracellular signaling domain.

The present application further provides nucleic acid molecules which encode various parts of the chimeric antigen receptor targeting CLDN18.2.

### Chimeric Antigen Receptor Targeting MSLN

In the present application, the chimeric antigen receptor targeting MSLN includes an antigen binding domain targeting MSLN.

In some embodiments, the antigen binding domain of the chimeric antigen receptor targeting MSLN includes at least one CDR in a heavy chain variable region VH, and the VH includes an amino acid sequence as set forth in any one of SEQ ID NO: 103, SEQ ID NO: 107, SEQ ID NO: 111, SEQ ID NO: 120, and SEQ ID NO: 155.

In some embodiments, the antigen binding domain of the chimeric antigen receptor targeting MSLN includes an HCDR3, and the HCDR3 includes an amino acid sequence as set forth in any one of SEQ ID NO: 101, SEQ ID NO: 106, SEQ ID NO: 110, SEQ ID NO: 119, and SEQ ID NO: 154.

In some embodiments, the antigen binding domain of the chimeric antigen receptor targeting MSLN includes an HCDR2, and the HCDR2 includes an amino acid sequence as set forth in any one of SEQ ID NO: 99, SEQ ID NO: 105, SEQ ID NO: 109, SEQ ID NO: 118, and SEQ ID NO: 153.

In some embodiments, the antigen binding domain of the chimeric antigen receptor targeting MSLN includes an HCDR1, and the HCDR1 includes an amino acid sequence as set forth in any one of SEQ ID NO: 97, SEQ ID NO: 104, SEQ ID NO: 108, SEQ ID NO: 117, and SEQ ID NO: 152.

In some embodiments, the antigen binding domain of the chimeric antigen receptor targeting MSLN includes an HCDR1, an HCDR2, and an HCDR3, and the HCDR1, HCDR2, and HCDR3 includes any one group of amino acid sequences selected from:
(1) the HCDR1 includes an amino acid sequence as set forth in SEQ ID NO: 97, the HCDR2 includes an amino acid sequence as set forth in SEQ ID NO: 99, and the HCDR3 includes an amino acid sequence as set forth in SEQ ID NO: 101;
(2) the HCDR1 includes an amino acid sequence as set forth in SEQ ID NO: 104, the HCDR2 includes an amino acid sequence as set forth in SEQ ID NO: 105, and the HCDR3 includes an amino acid sequence as set forth in SEQ ID NO: 106;
(3) the HCDR1 includes an amino acid sequence as set forth in SEQ ID NO: 108, the HCDR2 includes an amino acid sequence as set forth in SEQ ID NO: 109, and the HCDR3 includes an amino acid sequence as set forth in SEQ ID NO: 110;
(4) the HCDR1 includes an amino acid sequence as set forth in SEQ ID NO: 117, the HCDR2 includes an amino acid sequence as set forth in SEQ ID NO: 118, and the HCDR3 includes an amino acid sequence as set forth in SEQ ID NO: 119; and
(5) the HCDR1 includes an amino acid sequence as set forth in SEQ ID NO: 152, the HCDR2 includes an amino acid sequence as set forth in SEQ ID NO: 153, and the HCDR3 includes an amino acid sequence as set forth in SEQ ID NO: 154.

In some embodiments, the antigen binding domain of the chimeric antigen receptor targeting MSLN includes a VH, and the VH includes an amino acid sequence as set forth in any one of SEQ ID NO: 103, SEQ ID NO: 107, SEQ ID NO: 111, SEQ ID NO: 120, and SEQ ID NO: 155.

In some embodiments, the antigen binding domain of the chimeric antigen receptor targeting MSLN includes a VHH.

In some embodiments, the VHH includes an amino acid sequence as set forth in any one of SEQ ID NO: 103, SEQ ID NO: 107, and SEQ ID NO: 155.

In some embodiments, the antigen binding domain of the chimeric antigen receptor targeting MSLN includes an LCDR3, and the LCDR3 includes an amino acid sequence as set forth in SEQ ID NO: 123 or SEQ ID NO: 114.

In some embodiments, the antigen binding domain of the chimeric antigen receptor targeting MSLN includes an LCDR2, and the LCDR2 includes an amino acid sequence as set forth in SEQ ID NO: 113 or SEQ ID NO: 122.

In some embodiments, the antigen binding domain of the chimeric antigen receptor targeting MSLN includes an LCDR1, and the LCDR1 includes an amino acid sequence as set forth in SEQ ID NO: 112 or SEQ ID NO: 121.

In some embodiments, the antigen binding domain of the chimeric antigen receptor targeting MSLN includes an LCDR1, an LCDR2, and an LCDR3, and the LCDR1, LCDR2, and LCDR3 includes any one group of amino acid sequences selected from:
(1) the LCDR1 includes an amino acid sequence as set forth in SEQ ID NO: 112, the LCDR2 includes an amino acid sequence as set forth in SEQ ID NO: 113, and the LCDR3 includes an amino acid sequence as set forth in SEQ ID NO: 114; and
(2) the LCDR1 includes an amino acid sequence as set forth in SEQ ID NO: 121, the LCDR2 includes an amino acid sequence as set forth in SEQ ID NO: 122, and the LCDR3 includes an amino acid sequence as set forth in SEQ ID NO: 123.

In some embodiments, the antigen binding domain of the chimeric antigen receptor targeting MSLN includes a VL, and the VL includes an amino acid sequence as set forth in SEQ ID NO: 115 or SEQ ID NO: 124.

In some embodiments, the antigen binding domain of the chimeric antigen receptor targeting MSLN includes an scFv, and the scFv includes an amino acid sequence as set forth in SEQ ID NO: 116 or SEQ ID NO: 125.

In some embodiments, the chimeric antigen receptor targeting MSLN includes an intracellular signaling domain, and the intracellular signaling domain includes an intracellular signaling domain derived from one or more proteins selected from a group consisting of: CD3ζ, CD3δ, CD3γ, CD3ε, CD79a, CD79b, FcεRIγ, FcεRIβ, FcγRIIa, bovine leukemia virus gp30, Epstein-Barr virus (EBV) LMP2A, simian immunodeficiency virus PBj14 Nef, Kaposi's sarcoma-associated herpesvirus (HSKV), DAP10, DAP-12, and a domain containing at least one ITAM.

In some embodiments, the intracellular signaling domain is a signaling domain derived from CD3ζ.

In some embodiments, the intracellular signaling domain includes an amino acid sequence as set forth in SEQ ID NO: 16.

In some embodiments, the chimeric antigen receptor targeting MSLN includes a transmembrane region, and the transmembrane region includes a transmembrane region derived from one or more proteins selected from a group consisting of: CD8, CD28, 4-1BB, CD4, CD27, CD7, PD-1, TRAC, TRBC, CD3ε, CD3ζ, CTLA-4, LAG-3, CD5, ICOS, OX40, NKG2D, 2B4, CD244, FcεRIγ, BTLA, CD30, GITR, HVEM, DAP10, CD2, NKG2C, LIGHT, DAP12, CD40L, TIM1, CD226, DR3, CD45, CD80, CD86, CD9, CD16, CD22, CD33, CD37, CD64, CD134, CD137, CD154, and SLAM.

In some embodiments, the transmembrane region is a transmembrane region derived from CD28.

In some embodiments, the transmembrane region includes an amino acid sequence as set forth in SEQ ID NO: 14.

In some embodiments, the chimeric antigen receptor targeting MSLN includes a hinge region, and the hinge region includes a hinge region derived from one or more proteins selected from a group consisting of: CD28, IgG1, IgG4, IgD, 4-1BB, CD4, CD27, CD7, CD8, PD-1, ICOS, OX40, NKG2D, NKG2C, FcεRIγ, BTLA, GITR, DAP10, CD40L, TIM1, CD226, SLAM, CD30, and LIGHT.

In some embodiments, the hinge region is a hinge region derived from CD28.

In some embodiments, the hinge region includes an amino acid sequence as set forth in SEQ ID NO: 12.

In some embodiments, the chimeric antigen receptor targeting MSLN includes a signal peptide.

In some embodiments, the signal peptide is derived from a signal peptide of CD8 protein.

In some embodiments, the signal peptide includes an amino acid sequence as set forth in SEQ ID NO: 2.

In some embodiments, the chimeric antigen receptor targeting MSLN further includes a low-density lipoprotein receptor-related protein or a fragment thereof.

In some embodiments, the low-density lipoprotein receptor-related protein or the fragment thereof includes one or more selected from a group consisting of: low-density lipoprotein receptor-related proteins 1-12 and functional fragments thereof.

In some embodiments, the low-density lipoprotein receptor-related protein or the fragment thereof is low-density lipoprotein receptor-related proteins 5 and/or 6 or fragments thereof.

In some embodiments, the low-density lipoprotein receptor-related protein or the fragment thereof includes an amino acid sequence as set forth in SEQ ID NO: 18.

In some embodiments, the chimeric antigen receptor targeting MSLN does not include a costimulatory domain.

The present application further provides nucleic acid molecules which encode various parts of the chimeric antigen receptor targeting MSLN.

### Cell

In the present application, the expression level of the chimeric antigen receptor targeting CLDN18.2 included and/or expressed in the cell may be approximately 1:1 with that of the chimeric antigen receptor targeting MSLN.

In the present application, the expression level of the chimeric antigen receptor targeting CLDN18.2 included and/or expressed in the cell may be approximately 2:1 with that of the chimeric antigen receptor targeting MSLN.

In the present application, the cell may further include and/or express a low-density lipoprotein receptor-related protein or a fragment thereof.

In the present application, the low-density lipoprotein receptor-related protein or the fragment thereof may include one or more selected from a group consisting of: low-density lipoprotein receptor-related proteins 1-12 and functional fragments thereof.

In the present application, the low-density lipoprotein receptor-related protein or the fragment thereof may be low-density lipoprotein receptor-related proteins 5 and/or 6 or fragments thereof.

In the present application, the low-density lipoprotein receptor-related protein or the fragment thereof may include an amino acid sequence as set forth in SEQ ID NO: 18.

In the present application, the cell may include an immune effector cell.

In the present application, the cell may include T cells, B cells, natural killer cells (NK cells), macrophages, NKT cells, monocytes, dendritic cells, granulocytes, lymphocytes, leukocytes, peripheral blood mononuclear cells, embryonic stem cells, lymphoid progenitor cells and/or pluripotent stem cells.

For example, the cell may be a T cell.

### Expression Vector

In the present application, the expression vector may further include a nucleic acid sequence which encodes the low-density lipoprotein receptor-related protein or the fragment thereof.

In the present application, the low-density lipoprotein receptor-related protein or the fragment thereof may include one or more selected from a group consisting of: low-density lipoprotein receptor-related proteins 1-12 and functional fragments thereof.

In the present application, the low-density lipoprotein receptor-related protein or the fragment thereof may be low-density lipoprotein receptor-related proteins 5 and/or 6 or fragments thereof.

In the present application, the nucleic acid sequence which encodes the low-density lipoprotein receptor-related protein or the fragment thereof may include a nucleic acid sequence as set forth in SEQ ID NO: 18.

In the present application, the nucleic acid sequence which encodes the chimeric antigen receptor targeting CLDN18.2 may be linked to the nucleic acid sequence which encodes the chimeric antigen receptor targeting MSLN via a cleaving peptide.

For example, the cleaving peptide may be selected from a group consisting of: P2A, T2A, F2A, and E2A.

For example, the cleaving peptide includes an amino acid sequence as set forth in SEQ ID NO: 10. For example, the nucleic acid sequence which encodes the cleaving peptide may include a nucleic acid sequence as set forth in SEQ ID NO: 9.

In the present application, the expression vector may include at least one nucleic acid sequence which encodes the chimeric antigen receptor targeting CLDN18.2.

In the present application, the expression vector may include two nucleic acid sequences which encode the chimeric antigen receptor targeting CLDN18.2.

In the present application, the nucleic acid sequence which encodes the chimeric antigen receptor targeting CLDN18.2 and the nucleic acid sequence which encodes the chimeric antigen receptor targeting MSLN may be located in the same nucleic acid molecule.

In the present application, the nucleic acid sequence which encodes the chimeric antigen receptor targeting CLDN18.2, the nucleic acid sequence which encodes the chimeric antigen receptor targeting MSLN, and the nucleic acid sequence which encodes the low-density lipoprotein receptor-related protein or the fragment thereof are located in the same nucleic acid molecule.

In the present application, in the expression vector, a first nucleic acid sequence which encodes the chimeric antigen receptor targeting CLDN18.2, a second nucleic acid sequence which encodes the chimeric antigen receptor targeting CLDN18.2, a nucleic acid sequence which encodes the chimeric antigen receptor targeting MSLN, and a nucleic acid sequence which encodes the low-density lipoprotein receptor-related protein or the fragment thereof may be located in the same nucleic acid molecule.

In the present application, the expression vector may include, sequentially from 5′ end to 3′ end, a nucleic acid sequence which encodes the chimeric antigen receptor targeting CLDN18.2, a nucleic acid sequence which encodes the cleaving peptide, a nucleic acid sequence which encodes the chimeric antigen receptor targeting MSLN, a nucleic acid sequence which encodes the low-density lipoprotein receptor-related protein or the fragment thereof.

In the present application, the expression vector may include, sequentially from 5′ end to 3′ end, a nucleic acid sequence which encodes the chimeric antigen receptor targeting CLDN18.2, a nucleic acid sequence which encodes the cleaving peptide, a nucleic acid sequence which encodes the chimeric antigen receptor targeting CLDN18.2, a nucleic acid sequence which encodes the cleaving peptide, a nucleic acid sequence which encodes the chimeric antigen receptor targeting MSLN, a nucleic acid sequence which encodes the low-density lipoprotein receptor-related protein or the fragment thereof.

In the present application, the expression vector includes, sequentially from 5′ end to 3′ end, a nucleic acid sequence which encodes the signal peptide, a nucleic acid sequence which encodes CLDN18.2 scFv, a nucleic acid sequence which encodes the hinge region, a nucleic acid sequence which encodes the transmembrane region, a nucleic acid sequence which encodes the costimulatory domain, a nucleic acid sequence which encodes the cleaving peptide, a nucleic acid sequence which encodes the signal peptide, a nucleic acid sequence which encodes MSLN VHH, a nucleic acid sequence which encodes the hinge region, a nucleic acid sequence which encodes the transmembrane region, a nucleic acid sequence which encodes the intracellular signaling domain, and a nucleic acid sequence which encodes the low-density lipoprotein receptor-related protein or the fragment thereof.

In the present application, the expression vector includes, sequentially from 5′ end to 3′ end, a nucleic acid sequence which encodes the CD8 signal peptide, a nucleic acid sequence which encodes CLDN18.2 scFv, a nucleic acid sequence which encodes the CD8 hinge region, a nucleic acid sequence which encodes the CD8 transmembrane region, a nucleic acid sequence which encodes the 4-1BB costimulatory domain, a nucleic acid sequence which encodes the cleaving peptide, a nucleic acid sequence which encodes the CD8 signal peptide, a nucleic acid sequence which encodes MSLN VHH, a nucleic acid sequence which encodes the CD28 hinge region, a nucleic acid sequence which encodes the CD28 transmembrane region, a nucleic acid sequence which encodes the CDRζ intracellular signaling domain, and a nucleic acid sequence which encodes the low-density lipoprotein receptor-related protein or the fragment thereof.

In the present application, the expression vector includes, sequentially from 5′ end to 3′ end, a nucleic acid sequence which encodes the signal peptide, a nucleic acid sequence which encodes CLDN18.2 scFv, a nucleic acid sequence which encodes the hinge region, a nucleic acid sequence which encodes the transmembrane region, a nucleic acid sequence which encodes the costimulatory domain, a nucleic acid sequence which encodes the cleaving peptide, a nucleic acid sequence which encodes the signal peptide, a nucleic acid sequence which encodes CLDN18.2 scFv, a nucleic acid sequence which encodes the hinge region, a nucleic acid sequence which encodes the transmembrane region, a nucleic acid sequence which encodes the costimulatory domain, a nucleic acid sequence which encodes the cleaving peptide, a nucleic acid sequence which encodes the signal peptide, a nucleic acid sequence which encodes MSLN VHH, a nucleic acid sequence which encodes the hinge region, a nucleic acid sequence which encodes the transmembrane region, a nucleic acid sequence which encodes the intracellular signaling domain, and a nucleic acid sequence which encodes the low-density lipoprotein receptor-related protein or the fragment thereof.

In the present application, the expression vector includes, sequentially from 5′ end to 3′ end, a nucleic acid sequence which encodes the CD8 signal peptide, a nucleic acid sequence which encodes CLDN18.2 scFv, a nucleic acid sequence which encodes the CD8 hinge region, a nucleic acid sequence which encodes the CD8 transmembrane region, a nucleic acid sequence which encodes the 4-1BB costimulatory domain, a nucleic acid sequence which encodes the cleaving peptide, a nucleic acid sequence which encodes the CD8 signal peptide, a nucleic acid sequence which encodes CLDN18.2 scFv, a nucleic acid sequence which encodes the CD8 hinge region, a nucleic acid sequence which encodes the CD8 transmembrane region, a nucleic acid sequence which encodes the 4-1BB costimulatory domain, a nucleic acid sequence which encodes the cleaving peptide, a nucleic acid sequence which encodes the CD8 signal peptide, a nucleic acid sequence which encodes MSLN VHH, a nucleic acid sequence which encodes the CD28 hinge region, a nucleic acid sequence which encodes the CD28 transmembrane region, a nucleic acid sequence which encodes the CD3ζ intracellular signaling domain, and a nucleic acid sequence which encodes the low-density lipoprotein receptor-related protein or the fragment thereof.

### Use

In another aspect, the present application provides use of the cell and/or the expression vector in the preparation of a drug for preventing and/or treating a disease and/or a disorder.

In another aspect, the present application further provides use of the chimeric antigen receptor targeting CLDN18.2 and the chimeric antigen receptor targeting MSLN in the preparation of a drug for preventing and/or treating a disease and/or a disorder.

In another aspect, the present application provides a method for preventing and/or treating a disease and/or a disorder, which includes administering to a subject in need thereof the cell of the present application.

In another aspect, the present application provides the cell and/or the expression vector, which are used for preventing and/or treating a disease and/or a disorder.

In some embodiments, the disease and/or disorder includes a tumor.

In some embodiments, the tumor includes a solid tumor and/or a non-solid tumor.

In some embodiments, the tumor includes a tumor simultaneously expressing both antigens CLDN18.2 and MSLN.

In some embodiments, the tumor includes gastric cancer, pancreatic cancer, and/or gastroesophageal junction carcinoma.

Without intending to be limited by any theory, the following examples are only to illustrate various technical schemes of the present application, and not intended to limit the scope of the present application.

### EXAMPLES

### Example 1. Generation of CLDN18.2 Single-Chain Antibody and Identification of Antigen Binding Activity

In this example, cells with stable expression of human CLDN18.2, i.e., SP2/0-hCLDN18.2 cells, were obtained by lentivirus infection and flow sorting. Balb/c mice were subject to cell immunization, with SP2/0-hCLDN18.2 cells as the immunogen. Using mouse hybridoma technology, CLDN18.2-positive monoclonal cells were obtained by flow screening and subcloning, which were cultured in vitro to give IgG-like CLDN 18.2 mouse monoclonal antibodies. By sequencing in accordance with degenerate primer amplification method, the sequences of heavy chain variable region (V_{H}) and light chain variable region (V_{L}) were obtained. The heavy chain variable regions (V_{H}) and the light chain variable region (V_{L}) of the antibody were linked via a synthetic linker peptide (Linker) gene to form a recombinant gene, and the antibody expressed by the recombinant gene was the CLDN18.2 single-chain antibody, i.e., 5E6-scFv (SEQ ID NO: 38). At the same time, SP240-hCLDN18.1 cells with stable expression of human CLDN18.1 were obtained by lentiviral infection and flow sorting.

By means of flow cytometry, the specific binding activity of the 5E6-scFv single-chain antibody and the Standard antibody (SEQ ID NO:143) was identified. The SP2/0-hCLDN18.2 cells were counted, re-suspended in a flow buffer and adjusted to 1×10⁶/ml, which were added to a V-bottomed 96-well plate at 30 μl/well. A primary antibody was added at 30 μl/well, wherein the primary antibody was subject to 4-fold gradient dilution with the flow buffer to form 7 gradients starting from a working concentration of 80 μg/ml. APBS negative control was set for each antibody. The cells were incubated at 4° C. for 1 h, and washed once with the flow buffer. A secondary antibody goat F(ab′)2-anti-human IgG-Fc (DyLight® 650)(abcam, Cat #ab98593) was added at 30 μl/well. The cells were incubated at 4° C. for 30 min. and washed twice with the flow buffer, shaken to loose, and 25 μl/well of the flow buffer was added for loading. At the same time, the SP2/0-hCLDN18.1 cells were counted and plated. The primary antibody was subject to 3-fold gradient dilution to form a total of 5 concentration gradients starting from a working concentration of 10 μg/ml. The secondary antibody was goat F(ab′)2-anti-human IgG-Fc (DyLight® 650) (abcam, Cat #ab98593). The original data were introduced into GraphPad8.0 software for plotting and calculation. The results are shown in FIG. 1. The 5E6-scFv single-chain antibody can specifically bind to human CLDN18.2, which is consistent with the Standard Antibody.

### Example 2. Identification of Binding Activity of CLDN18.2 Single-Chain Antibody to Non-Target Cells

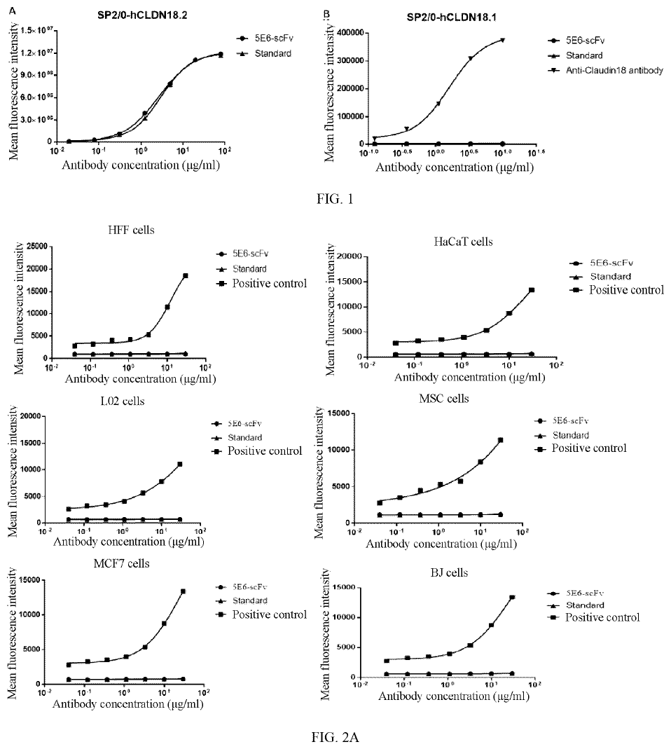

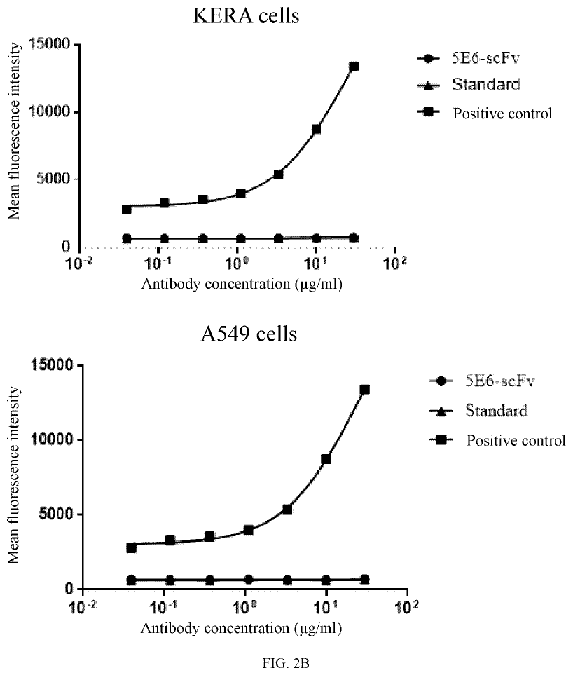

In this example, the binding activity of the 3E6-scFv single-chain antibody to various non-target cells in human tissues was identified by fluorescence-activated cell sorting (FACS). The cells were human foreskin fibroblasts (HFF), human immortal epidermal cells (HaCaT), human normal liver cells (LO2), mesenchymal stem cells (MSC), human normal epidermal cells (KERA), adenocarcinoma human alveolar basal epithelium cells (A549), human breast cancer cells (MCF-7), human skin fibroblasts (BJ), respectively. The antibody was subject to 3-fold gradient dilution to form a total of 7 concentration gradients starting from a working concentration of 30 μg/ml. The secondary antibody was goat F(ab′)2-anti-human IgG-Fc (DyLight® 650) (abcam, Cat #ab98593). The cells were incubated, then centrifuged, re-suspended in the flow buffer, and loaded. The results were introduced into GraphPad8.0 software for plotting. The results are shown in FIG. 2. The 5E6-scFv single-chain antibody does not specifically bind to the various non-target cells in human tissues, which is consistent with the Standard antibody, primarily verifying its specificity to the tumor targets, namely, there is no “off-target effect” phenomenon.

### Example 3 Stable Expression of CLDN18.2-Specific CAR in T Cells

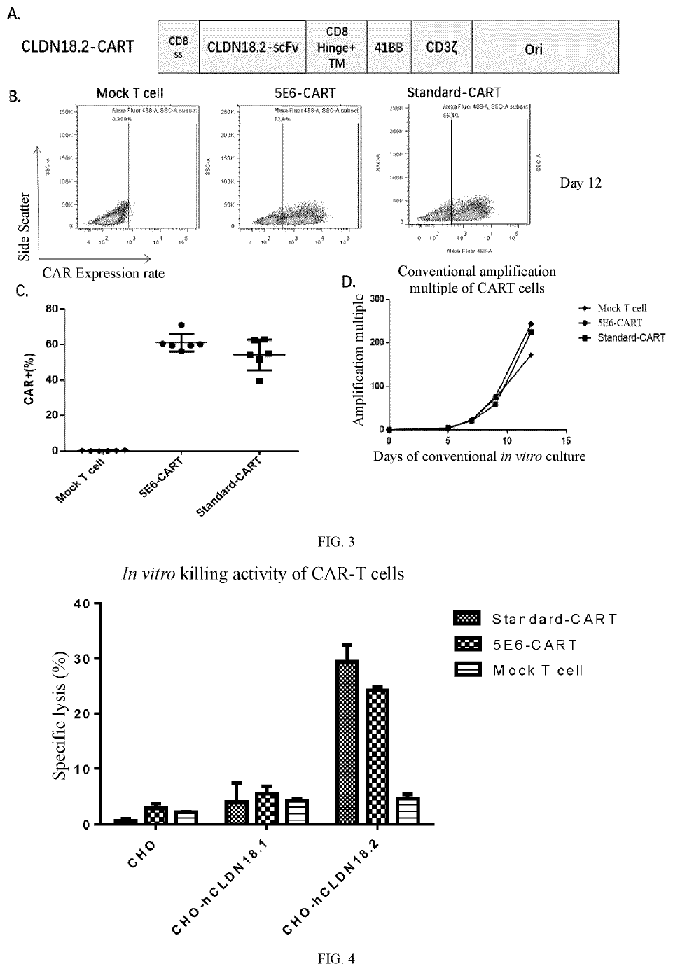

As shown in FIG. 3A, the CLDN18.2-specific CAR structure in this example is composed of a human CD8 signal peptide (SEQ ID NO: 2), an anti-human CLDN18.2 single-chain antibody (SEQ ID NO: 38), a human CD8 hinge region (SEQ ID NO: 4), a human CD8 transmembrane region (SEQ ID NO: 6), a human 4-1BB intracellular costimulatory domain (SEQ ID NO: 8), a human CD3ζ intracellular activation domain (SEQ ID NO: 16) and an added new Ori element (SEQ ID NO: 18). First, this example investigated the expression of CLDN18.2-specific CAR in human T cells and the amplification multiple of CART cells under conventional culture conditions in vitro. The specific method was as follows:
1) Human PBMC cells cryopreserved in liquid nitrogen were resuscitated in a 37° C. water bath, and centrifuged, re-suspended and rinsed three times using a system of 11 mL of PBS+1 mL of PBMC (500 g, 5 min; 400 g, 3 min; 300 g, 5 min). The rinsed human PBMC cells were mixed with CD3 MicroBeads, human (Miltenyi, 130-050-101), and subject to CD3 positive sorting with a magnetic grate, i.e., separating and retaining CD3⁺ T cells. The CD3⁺ T cells were re-suspended in a medium containing 4% FBS+X-VIVO (Lonza Company)+20 ng/ml of Factor 1+10 ng/ml of Factor 2 to a cell density of 1×10⁶ cells/mil. CD3/CD28 magnetic beads (Thermo Fisher 40203D, the magnetic beads were washed twice with the medium, absorbed to the magnetic grate, and left for 1 min) were added at a ratio of 1:3 (cells:magnetic beads) to activate the T cells. The T cells were added to the magnetic beads, mixed well, and supplemented with medium to 700 μl for each well of the 12-well plate. The cell number was 7×10⁵/well, the density was 1×10⁶ cells/ml, and the day of sorting was recorded as Day 0.
2) The activated T cells were cultured in a 37° C., CO₂ incubator for 20 h. Then, a corresponding virus supernatant was added at a multiplicity of infection (MOI) of 4. Polybrene was added to a final concentration of 10 μg/ml. After mixed well by pipetting, the cells were centrifuged at 1200 rpm in a horizontal centrifuge for 1 h. The well plate was placed back to the 37° C. CO₂ incubator, and cultured for 24 h. The day of infection was recorded as Day 1.
3) 24 h later, the cells in each well of the 12-well plate were repeatedly pipetted to uniform, transferred to a 1.5 mL EP tube, and centrifuged at 400 g for 3 min. The supernatant was removed, and the cells were re-suspended in fresh X-VIVO complete medium (1 mL) to a cell density of 7×10⁵ cells/mi and cultured in the 37° C., CO₂ incubator. The medium was replenished once it turned yellow. The cells were counted every 2 days, and replenished with fresh X-VIVO complete medium to adjust the cell density to 7×10⁵ cells/mi. The counting results were recorded and statistically analyzed, and plotted by GraphPad8.0 software to calculate the amplification multiple of the CART cells under conventional culture.
4) The CAR-T cells cultured for 9-14 days were detected for their cellular positive rate. The virus used to infect the cells carried Myc-tag so that the Myc positive rate of the cells after virus infection could be detected by a flow cytometer so as to obtain the expression positive rate of CAR. The directly labeled detection antibody used was Myc-Tag(9B11) Mouse mAb (Alexa Fluor® 488 Conjugate)(Cell Signaling, 2279S).

In accordance with MOI=4, the cells were infected with human CLDN18.2 sequence viruses, including the 5E6 sequence and Standard sequence viruses. The results are shown in FIG. 3B. On the 12^{th} day of the CART cell culture in vitro, the CAR positive rate was detected by flow cytometry. The positive rate of 5E6-CART was 72.6% and the positive rate of Positive control CAR-T was 65.4%, in this example, a total of 6 different PBMC donors were used to verify the CAR positive rate. FIG. 3C shows that the positive rate of 5E6-CART in the 6 different PBMC donors remains around 60%, which is consistent with that of the Standard-CART. FIG. 3D shows that the cumulative amplification multiple of 5E6-CART cells conventionally cultured in vitro far 12 days is about 240, which is not significantly different from the amplification multiple of Standard-CART.

### Example 4. In Vitro Specific Killing of Target Cells by CLDN18.2-Specific CAR

In this example, CO-hCLDN18.2 cells with stably high expression of human CLDN18.2 and CHO-hCLDN18.1 cells with stably high expression of human CLDN18.1 were first obtained by lentiviral infection and flow sorting. Further, the in vitro specific killing ability of the CAR-T cells was evaluated by LHD method using a cytotoxicity detection kit (Promega. Cat #G1780) in this example by the following steps.

The CAR-T and Mock T cells that had been conventionally cultured for 9 days in Example 3 were centrifuged and re-suspended in a blank X-VIVO medium to a cell density of 1×10⁵/ml, respectively. The target cells comprise three cells, that are, CHO, CHO-hCLDN18.2 and CHO-hCLDN18.1. The three target cells were digested and counted respectively, and then re-suspended in a blank X-VIVO medium to a cell density of 5×10⁵/ml. Then, a volume system with 100 μl of target cell suspension+100 μl of CAR-T/Mock T cell suspension in each well was mixed and added into a sterile v-bottomed 96-well plate. A control well was set according to the requirements of the kit. The resultant mixture was co-incubated in a 37° C. incubator for 24 h, and detected by a microplate reader to record the absorbance at 490 nM. The lysis percentage of the target cells was calculated using the formula given by the kit, and the data were analyzed and processed by Graphpad prism 8. The results are shown in FIG. 4. The 5E6-CART specifically induces the lysis of CLDN18.2-positive target cells in vitro, and produces no lysis effect on negative cells and CLDN18.1-positive cells, which is consistent with the Standard-CART, indicating that the 5E6-CART cells has high human CLDN18-specific killing activity on the target cell in vitro.

### Example 5. Factor Secretion of CLDN18.2-Specific CAR

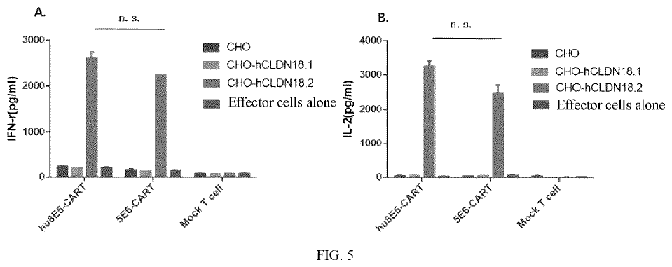

Human IFN-γ ELISA kit (R&D, DY285B) and Human IL-2 ELISA kit (R&D, DY202) were respectively used in this example to analyze the effects of CAR-T on the secretion of IFN-γ and IL-2 during the process of killing the target cells. The details are as follows. The target cells with high expression of CLDN18.2 (CHO-hCLDN18.2), the control cells with high expression of CLDN18.1 (CHO-hCLDN18.1) and the negative cells CHO were inoculated into a sterile 9-well plate at 1×10⁴ cells per well, respectively, and effector cells such as CAR-T cells (5E6-CART and Standard-CART) and unmodified T cells (Mock T cells) were added into the target cells at ratio of effector cells (Effector):target cells (Target)=5:1. After incubation for 24 h, the supernatant was sampled to detect the contents of IL-2 and IFN-γ by the enzyme-linked immunoassay (ELISA) with operation according to the instructions of the kit. The results are shown in FIGS. 5A and 5B. The 5E6-CART has a higher factor secretion level when co-incubated with CLDN18.2-positive cells, and has no significant factor secretion when co-incubated with negative cells and CLDN18.1-positive cells, which is consistent with the Standard-CART. Therefore, 5E6-CART cells have specific cytokine-inducing effect on the tumor cells with high expression of CLDN18.2.

### Example 6. Antitumor Effect of CLDN18.2-Specific CAR-T Cells in Mouse Colon Cancer CDX Models

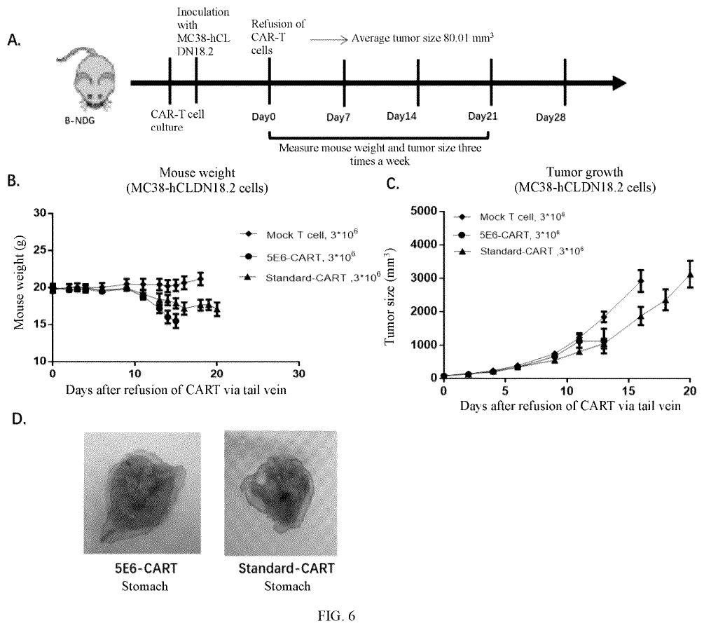

In this example, human CLDN18.2 gene was first introduced into MC38 cells by lentivirus, and then mouse colon cancer cells with high expression of human CLDN182. i.e., MC38-hCLDN18.2 cells, were sorted by a flow sorter. A mouse colon cancer tumor model was constructed by subcutaneous injection using heavily B-NDG-immunized female mice to verify the antitumor effect of the CLDN18.2-specific CAR-T cells in mice. The MC38-hCLDN18.2 cells were inoculated at a dose of 1.5×10⁶/mouse. On Day 8 after tumor inoculation of mice, the mice having tumor size of 41.68-120.6 mm³ (average tumor size of 80.01 mm³) were randomly divided into 3 groups, with 8 mice per group. The CAR-T (or Mock T) cells that had been cultured for 10 days were re-infused via the tail vein, wherein the refusion amount of CAR-T was 3*10⁶ cells/mouse in the 5E6-CART and Standard-CART groups, and the refusion amount was 1*10⁷ cells/mouse in the blank control Mock T cell group. The mice were detected three times a week for their tumor size and weight, and observed for 4 weeks, as shown in FIG. 6A. After the refusion of CAR-T (or Mock T) cells to the mice via the tail vein, in the 5E6-CART group, the mice had more than 10% of weight loss on D13, individual mice exhibited −34.51% of relative change in weight on D15, and the first death of mouse occurred on D13; and in the Standard-CART group, the mice had more than 10% of weight loss on D16; individual mice exhibited −36.93% of relative change in weight on D16; the first death of mouse occurred on D18, and the experiments ended at PG-D22 due to the serious weight loss of mice, and no significant antitumor effect was observed at the end of the experiment, see FIG. 6C (the end point was defined as mouse tumor size >3000 mm). The mice were euthanized and dissected. It was found that mice in both the 5E6-CART group and the Standard-CART group exhibited abnormal gastric damage and bleeding, as shown in FIG. 6D. To sum up, the mice in the 5E6-CART group showed intolerance problems and obvious side effects in this tumor model, which were specifically manifested as weight loss, back hair standing, curling up motionlessly, gastric bleeding, or the like; and the mice in the Mock T group showed tolerance and normal weight, etc.

In summary, 5E6-scFv is a single-chain antibody obtained by screening and identification via mouse hybridoma technology. The 5E6-scfv antibody can specifically bind to human CLDN18.2 positive cells and has high specificity for CLDN18.2. The 5E6-scFv single-chain antibody does not bind non-specifically to a variety of non-target cells in human tissues. The 5E6-CART is a second-generation CART structure that can be stably expressed and proliferated in T cells. The 5E6-CART has highly specific cell killing activity on CLDN18.2 in vitro and can specifically induce cytokine secretion. The 5E6-CART shows a certain toxicity in efficacy experiments on tumor-bearing mice, which is manifested as weight loss and gastric bleeding in mice, indicating to an extent that 5E6-CART has poor safety in mice. Therefore, this example discovers and combines new tumor targets by investigation, and designs a new CAR molecular structure which has improved in vivo safety while retaining the effectiveness of 5E6, and is designated as OriCAR-388.

### Example 7. Construction of OriCAR-388 Lentiviral Vector and Viral Packaging

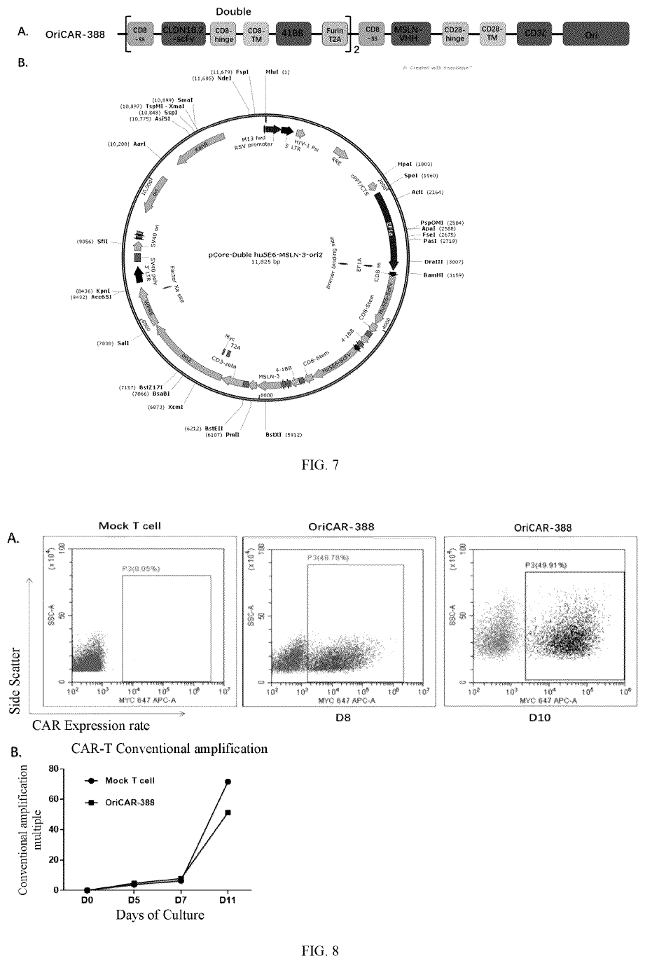

The tumor target MSLN is highly expressed in 50-55% of gastric cancer patients and 80-85% of pancreatic cancer patients, and can be used as gastric cancer target for research. In normal human tissues, the target MSLN has high expression in trachea, fallopian tube, blastoderm and tonsils, moderate expression in nasopharynx and endometrium, low expression in oral mucosa, uterus and skin, and no expression in normal lung, stomach, liver, pancreas, and gallbladder, which does not substantially overlap with the expression distribution of CLDN182 in normal human tissues, and is an ideal choice of dual CAR-T target. To improve the safety of 5E6-CART, we designed a molecular structure of dual CAR-T by linking a human CD8 signal peptide (SEQ ID NO: 2), an anti-CLDN18.2 scFv antigen-binding domain in Example 1 (5E6-scFv, SEQ ID NO: 38), a human CD8 hinge region (SEQ ID NO: 4), a human CD8 transmembrane region (SEQ ID NO: 6), a human 4-1BB intracellular costimulatory domain (SEQ ID NO: 8), and FurinT2A self-cleaving peptide to form Fragment 1; linking a human CD8 signal peptide (SEQ ID NO: 2), an anti-human MSLN VHH antigen binding domain (SEQ ID NO: 103), a human CD28 hinge region (SEQ ID NO: 12), a human CD28 transmembrane region (SEQ ID NO; 14), and a human CD3, intracellular signaling domain (SEQ ID NO: 16) to form Fragment 2, designating the Ori element as Fragment 3, and then linking a combination of “Fragment 1+Fragment 1+Fragment 2+Fragment 3” together. The schematic structural view is shown in FIG. 7A. The linked fragments were constructed into the in-house pCore four-plasmid lentiviral expression vector by homologous recombination. The constructed vector was sequenced, and then subject to plasmid extraction. The extracted plasmids were filtered, sterilized, and detected for their concentration. The vector map is shown in FIG. 78.

In this example, the lentiviral expression vectors were subject to lentiviral packaging with a four-plasmid system as follows: 1) The four-plasmid system composed of lentiviral expression vector plasmid and helper plasmids PRH1/PMH2/PVH3 were mixed with a FUGENE HD transfection reagent (Promega Company, FuGENE HD:Plasmid=3:1), added into a volume of opti-MEM medium (Gibco), mixed well, and left for 15 min. 2) The mixed liquor was evenly added dropwise to a 6-well plate coated with 293T cells, mixed gently, and immediately cultured in a CO incubator at 37° C. for 18 h. 3) After 18 h, the medium was replaced with fresh medium, 1.5 mL of DMEM medium containing 5% FBS (preheated at 37° C. for 30 min in advance) was gently added dropwise, the mixture was cultured overnight for additional 24 h, and then the lentivirus culture supernatant was collected for virus titer detection. 4) 10 μl of the virus supernatant was taken to infect 293T cells, cultured in a 37° C., 5% CO₂ incubator for 72 h, and then an appropriate amount of virus-infected 293T cells were detected by flow cytometry for the virus titer, wherein the detection antibody used was myc-tag (9B11) mouse mAb (Alexa R 647)(Cell signaling, 2233S), and the virus titer was calculated by the formula of: titer=number of infected cells*positive rate/volume of infected virus (ml).

### Example 8. Transfection of T Cells by Lentivirus, Stable Expression of CAR In T Cells, and Stable Proliferation of CAR-T Cells

The PBMCs were resuscitated, positive sorted and activated according to the method shown in Example 3 to obtain the activated CD3-positive T cells. Corresponding volume of virus supernatant was added at a multiplicity of infection (MOI) of 3, and polybrene was added to a final concentration of 10 μg/ml (1:1000). The cells were mixed well by pipetting, and centrifuged at 1200 rpm in a horizontal centrifuge for 1 h. Then, the well plate was placed back into the 37° C., CO₂ incubator, and cultured for 18-24 h. The day of virus infection was recorded as Day 1. The next day, virus was removed, and the medium was replaced with fresh medium. Then, the well plate was placed in the 37° C. CO₂ incubator for further culture. The medium was replenished with complete X-VIVO medium once it turned yellow, and the medium preparation was the same as that in Example 3. CAR-T cells were detected for their CAR-positive rates on Day 8 and Day 10 of conventional culture, where the detection antibody was myc-tag (9B11) mouse mAb (Alexa R 647)(Cell signaling, 2233S), while Mock T was set as the negative control. The results are shown in FIG. 8A. The OriCAR-388 can be stably expressed in T cells, and the CAR expression rate is maintained at around 49%. The CAR-T cells and the Mock T cells were counted on Day 5, Day 7 and Day 11 of conventional CAR-T cell culture. After statistics, the data were plotted and analyzed by Graphpad prism8.0 drawing software. The results are shown in FIG. 8B. The OriCAR-388 T cells can be stably proliferated in vitro, and the cumulative amplification multiple is around 50 after 11 days of in vitro culture.

### Example 9. Detection of In Vitro Killing of OriCAR-388 T Cells by Luciferase Method

The conventional culture of CAR-T and blank T cells was carried out according to the method shown in Example 8. The CAR-T cells (OriCAR-388 T cells and 5E6-CART cells) that have been conventionally cultured in vitro for 12 days and untransfected blank T cells (Mock T) were centrifuged and re-suspended in blank X-VIXO medium (Lonza) to a cell density of 1.33*10⁵/ml. The CHO-NFkB-hCLDN18.2 is a cell with high expression of both human CLDN18.2 and luciferase and obtained by introducing the NFkB luciferase reporter gene by molecular construction based on the construction of Example 4, followed by subcloning, which is used as CLDN18.2 single-target target cell for research. The NUGC4 cell is a human gastric cancer cell that naturally express human CLDN18.2 and human MSLN under conventional in vim culture conditions. The NUGC4-NFkB cell is constructed by introducing the NFkB luciferase reporter gene, which is used as dual-target positive target cell for research. The OVCAR3-NFkB is a human ovarian cancer cell that naturally highly expresses human MSLN protein under conventional in vitro culture conditions. The OVCAR3-NFkB cell is constructed by introducing the NFkB luciferase reporter gene and used as MSLN single-target positive target cell for research. The three target cells were digested and counted respectively, and re-suspended in blank X-VIXO medium (Lonza) to a cell density of 1.33*10⁵/ml. A volume system of “75 μl of target cell suspension+75 μl of CAR-T/Mock T cell suspension” was added into a white-bottomed, opaque, sterile 96-well plate (Nunclon Delta Surface CAT: 136101), that is, at an effector-to-target ratio of E:T=1:1. At the same time, a well for incubating the target cells alone, i.e., “75 μl of target cell suspension+75 μl of blank X-VIVO medium”, was set. The %-well plate was cultured in a 37° C., CO₂ incubator for 18-24 h. Then, an equal volume of luciferase reaction substrate was added following the operating requirements of the ONE Glo Luciferase Assay System kit (promega, E6120), and the cells in the 96-well plate were detected for their luciferase content within 5 min at room temperature in the dark, by a TECAN multi-function microplate reader Spark (TECAN, Switzerland) The killing efficiency was calculated according to the following formula: Killing rate %=(1−RLU experimental well/RU only target cell well)×100%.

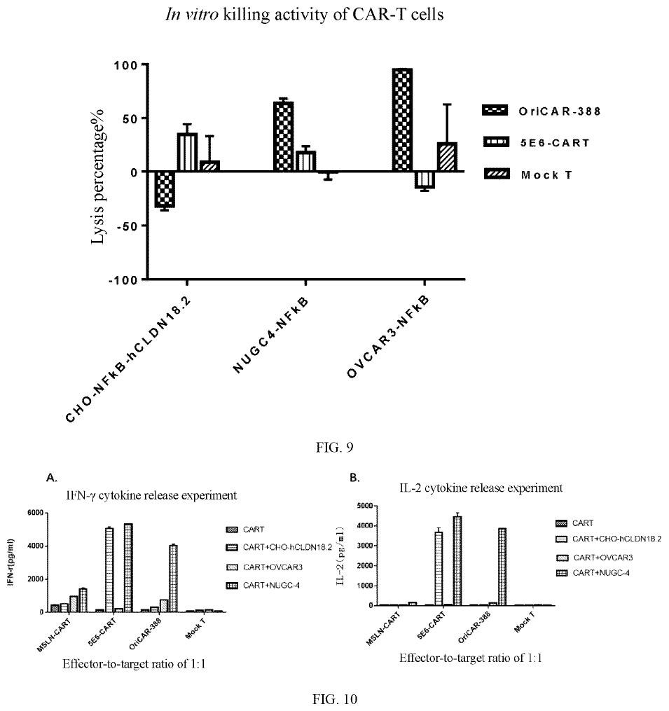

The results are shown in FIG. 9. For CHO-NFkB-hCLDN18.2 cells, the OriCAR-388 T cells have no killing activity, and the 5E6-CART cells have strong killing activity. For OVCAR3-NFkB cells, due to their high expression of MSLN protein, the OriCAR-388 T cells have strong killing activity, and the 5E6-CART cells have no killing activity. For NUGC4-NFkB cells expressing both the MSLN protein and the CLDN18.2 protein, the OriCAR-388 T cells have strong killing activity, and the 5E6-CART cells have moderate killing activity.

### Example 10. Determination of Cytokine Secretion of OriCAR-3 T Cells by ELISA

In this example, CHO-hCLDN18.2 was selected as the human CLDN18.2 single-positive target cell, OVCAR-3 as the human MSLN single-positive target cell, NUGC-4 as the MSLN/CLDN18.2 double-positive target cell, and the effector cells were CAR-T cells and Mock T cells that have been cultured in vitro for 8 days. The effector cells were centrifuged to remove the supernatant, diluted with blank X-VIVO to 2*10⁵/ml, and 100 μl was sampled from each well for use. The target-free well (that is, CAR-T cells which were not co-cultured with the target cells) and the wells in which the effector cells were co-incubated with target cells at an effector-to-target ratio of 1:1 were inoculated in a flat-bottomed 96-well plate, and incubated for 20 h. Then, the supernatant was aspirated and detected for the content of IL-2 and IFN-γ by a R&D Cytokine Kit (R&D, DY285B and R&D. DY202).

The results are shown in FIG. 10. MSLN-CART cells secret factors only when co-incubated with OVCAR-3 or NUGC-4 cells, and the secretion level is relatively low. 5E6-CART cells secrete a large number of IFN-γ and IL-2 cytokines when co-incubated with CHO-hCLDN18.2 or NUGC-4 cells. And OriCAR-388 T cells substantially do not secret IFN-γ and IL-2 cytokines when co-incubated with CHO-hCLDN18.2, and exhibit no difference in the factor secretion level from the only-CAR-T incubation well. OriCAR-388 T cells can secrete a small amount of IFN-γ and IL-2 cytokines when co-incubated with OVCAR-3, wherein the IL-2 secretion level is significantly higher than that of the only-CAR-T incubation cells. And OriCAR-388 T cells have an extremely significant improvement in the IL-2 and IFN-γ cytokine secretion level when co-incubated with NUGC-4 (p<0.0001). This also excellently verifies the design concept of the dual CAR-T structure, that is: the MSLN-VHH is directly linked to the CD3ζ intracellular signaling domain, which can independently transduce the signal of the extracellular MSLN antigen-binding domain and activate the secretion of cytokines; CLDN18.2-scFv is directly linked to the 4-1BB costimulatory domain, which cannot independently accomplish the signal transduction of the extracellular CLDN18.2 antigen-binding domain, thereby failing to activate the secretion of cytokines; and by linking the two elements in parallel via a self-cleaving polypeptide T2A, in theory, only when CLDN18.2 and MSLN antigens coexist, CD3ζ can specifically transduce dual-target signals at the same time, while the 4-1BB domain stimulates the secretion of more cytokines.

### Example 11. Antitumor Effect of OriCAR-388 T Cells in Human Gastric Cancer CDX Model

The NUGC-4 cell is a human gastric cancer cell naturally co-expressing human CLDN18.2 and human MSLN proteins under conventional in vitro culture conditions, which can be used as target cells co-expressing dual targets for research of the in viva efficacy of the OriCAR-388 T cells. Specifically, the mouse strain used was severely immunodeficient B-NDG mice. All the mice were fed in cage for one week of adaptation, and then subcutaneously inoculated with tumors (NUGC-4 cells, 3*10⁶ cells/mouse). 43 days after tumor inoculation, mice with average tumor size of 976 mm³ were randomly divided into 3 groups, with 5 mice per group. The OriCAR-388 T cells, 5E6-CART cells and Mock T cells that had been cultured for 11 days were re-infused via the tail vein (the day of refusion was recorded as Day 0), as shown in FIG. 11A, wherein the Mock T cells were re-infused at a dose of 1*10⁷ cells/mouse (labeled as G1 group: Mock T cell, 1*10⁷), the OriCAR-388 T cells were re-infused at high- and low-dose, respectively, i.e., 1*10⁷ cells/mouse (labeled as G2 group: OriCAR-388 T, 1*10⁷) and 3*10⁶ cells/mouse (labeled as Group G3: OriCAR-388 T, 3*10⁶). The mice were measured for their tumor size and weight three times a week, and observed for about 4 weeks. That is, the mice were euthanized to end the experiment after 33 days. The results are shown in FIG. 11B. The mice in the 5E6-CART group began to exhibit weight loss since the 8^{th} day after the CART refusion, while in the Mock T cell group, OriCAR-388 Thigh-dose group and OriCAR-388 T low-dose group, the mice was normal in weight, had bright fur, and were normal in their behavior, food and water intake, and color of feces and urine throughout the experimental observation period (observed until Day 33 after the CAR-T refusion). Compared with the Mock T cell group, both the OriCAR-388 T cell high-dose and low-dose groups showed extremely significant anti-tumor activity (p<0.0001), as shown in FIG. 11C (the endpoint was defined as mouse tumor size >3000 mm³). On Day 33 after the CART refusion, the average tumor size of the mice in the high-dose OriCAR-388 T cell group decreased to 273.10 mm³, and the average tumor size of the mice in the low-dose OriCAR-388 T cell group decreased to 310.44 mm. It was confirmed that the OriCAR-388 T cells had a tumor elimination effect and good safety in mice.

To sum up, OriCAR-388 can be stably expressed and proliferated in T cells, the CAR expression rate is maintained at around 49%, and the amplification multiple of CART cells conventionally cultured in wino for 11 days is about 50. OriCAR-388 T cells have strong killing activity only on the NUGC-4 double target positive cells, and have no or low killing activity on the CLDN18.2 single positive target cells or the MSLN single positive target cells. OriCAR-388 T cells substantially do not secrete IFN-γ and IL-2 cytokines when co-incubated with CHO-hCLDN18.2 cells, and secrete a small amount of IFN-γ and IL-2 cytokines when co-incubated with OVCAR-3, while OriCAR-388 T cells exhibited extremely improved secretion levels of IL-2 and IFN-γ cytokines when co-incubated with NUGC-4 cells. The immunodeficient mice were subcutaneously inoculated with tumor NUGC-4 cells, and subject to CAR-T cell refusion when the average tumor size reached 976 mm³. The purpose was to obtain possible toxic side effects under full and repeated activation of CAR-T by using this super-large tumor model to simulate the high in vivo tumor load. It was observed in the experiment that in the OriCAR-388 T high- and low-dose groups, the mice did not have abnormality in weight, had bright fur, and were normal in their behavior, food and water intake, and color of feces and urines throughout the experimental observation period. Both the OriCAR-388 T cell high- and low-dose groups showed extremely significant anti-tumor activity. On Day 33 after the CART refusion, the average tumor size of mice in the high-dose OriCAR-388 T cell group decreased to 273.10 mm³ and the average tumor size of mice in the low-dose OriCAR-388 T cell group decreased to 310.44 mm³, confirming that the OriCAR-388 T cells had a certain antitumor effect and good safety in mice. However, the 5E6-CART targeting the Claudin18.2 single target shows serious toxicity in an efficacy experiment on tumor-bearing mice, manifested as dramatic weight loss, gastric bleeding, etc.

### Example 12. Toxicology Experiment of OriCAR-388 T Celts in Mice

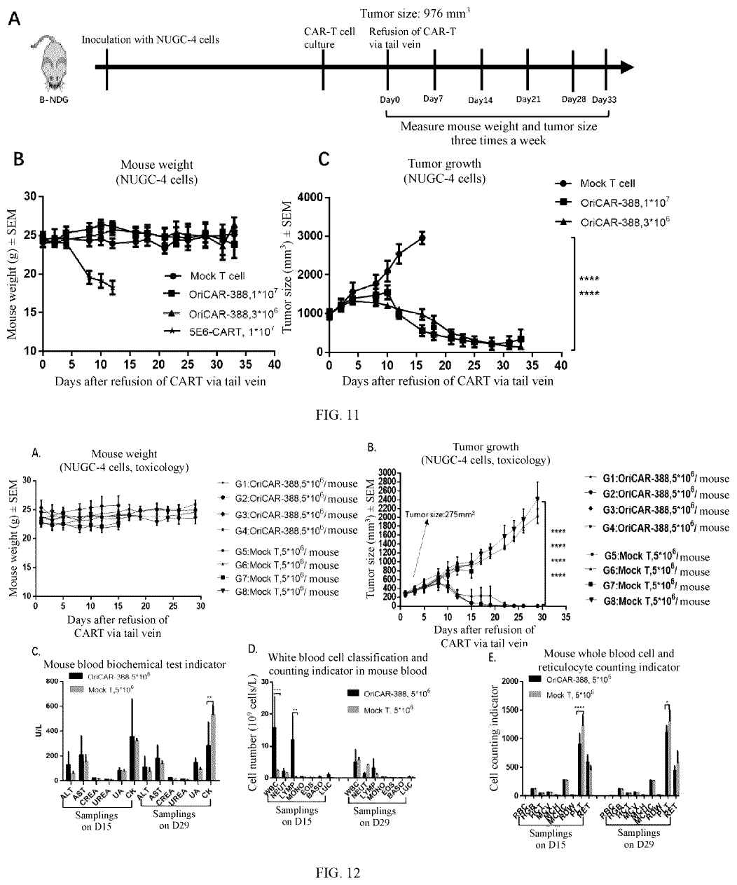

To further verify the effectiveness and safety of OriCAR-388 T, this example conducted a single-dose toxicity test in a tumor-bearing mouse model, i.e., mouse toxicology test. The details are as follows: 12 female B-NDG mice were subcutaneously inoculated with tumor NUGC-4 cells, and received CAR-T cell refusion when the tumor size reached about 270 mm³. The mice were evenly divided into 4 groups with the refusion doses of 5*10⁶ cells/mouse for Mock T and 5*10⁶ cells/mouse for OriCAR-388 T. After the refusion, the mice were continuously observed to PG-D29. The specific grouping scheme is shown in Table 1. The weight of the mice is shown in FIG. 12A. For the four groups G3/G6/G8/G10, the mice were euthanized on Day 15 after the refusion of CAR-T or Mock T cells (PG-D15) via the tail vein, and dissected for harvesting the organs. Thus, the weight of the mice was only continuously observed until PG-D15. During the observation period, the weight of the mice remained normal, and them was no significant weight loss. For the four groups of G4/G7/G9/G11, the mice were euthanized on PG-D29, and dissected for harvesting the organs. During the observation period, the weight of the mice also remained normal, and there was no significant weight loss. When the average tumor size of the mice reached 275 mm³ the mice were divided into groups and received the refusion of CAR-T/Mock T cells on the day. During the observation period, the data of tumor growth in mice were analyzed as shown FIG. 12B. All the mice in the OriCAR-389 refusion group exhibited extremely significant difference (p<0.0001) in tumor size at the end of tumor monitoring as compared with the tumor si: of the mice in the Mock T group measured at the same time, showing a significant anti-tumor activity. On PG-D15 and PG-D29, the mice were detected for blood biochemical indicators, white blood cell differential counting indicators, whole blood cell and reticulocyte counting indicators, and the detection results were statistically analyzed. The mouse blood biochemical test indicators are shown in FIG. 12C. Among all the detection indicators of the D15 sampling results, the ALT/AST level of the OriCAR-388 group was higher than that of the Mock T group, but there was no statistical difference (p>0.05). Among all the detection indicators of the D29 sampling results, the ALT/AST level of the OriCAR-388 group was still higher than that of the Mock T group, but there was also no statistical difference (p>405). The CK level of the OriCAR-388 group was significantly lower than that of the Mock T group (p<0.01), and this significant difference did not have a clinical significance. The white blood cell classification and counting indicators in mouse blood are shown in FIG. 12D. In the D15 sampling results, the cell counting level of LYMP (lymphocytes) of the OriCAR-388 group was significantly higher than that of the Mock T group (p<0.01), and in the D29 sampling test, the cell count level of LYMP (lymphocytes) of the OriCAR-388 group was still higher than that of the Mock T group, which was slowed down, and there was no statistical difference (p>0.05). The detection results of mouse whole blood cell and reticulocyte counting indicators were shown in FIG. 12E. When sampled on Day 15, the PLT level of the OriCAR-388 group was extremely significantly lower than that of the Mock T group (p<0.0001), while when sampled on D29, there was still a significant difference in the PLT level of the OriCAR-388 group compared to that of the Mock T group, but the difference was slowly reduced (p<0.05). This example further confirms the effectiveness and safety of OriCAR-388 T cells in mice.

TABLE 1

| Group- | Tumor | CAR-T cell | Sampling | |
|---|---|---|---|---|
| ing | cells | refusion | time | Detection Indicator |
| G1 | NUGC-4, | OriCAR-388 | PG-D15 | Blood biochemistry testing, copy number |
| | 3*10⁶ | T cells | | detection in various tissues |
| G2 | cells/ | OriCAR-388 | PG-D29 | Blood biochemistry testing, copy number |
| | mouse | T cells | | detection in various tissues |
| G3 | | OriCAR-388 | PG-D15 | Blood cells, flow cytometry (CD3/CD4/CD8), |
| | | T cells | | copy number detection |
| G4 | | OriCAR-388 | PG-D29 | Blood cells, flow cytometry (CD3/CD4/CD8), |
| | | T cells | | copy number detection |
| G5 | | Mock T cells | PG-D15 | Blood cells, flow cytometry (CD3/CD4/CD8), |
| | | | | copy number detection |
| G6 | | Mock T cells | PG-D29 | Blood cells, flow cytometry (CD3/CD4/CD8), |
| | | | | copy number detection |
| G7 | | Mock T cells | PG-D15 | Blood biochemistry testing, copy number |
| | | | | detection in various tissues |
| G8 | | Mock T cells | PG-D29 | Blood biochemistry testing, copy number |
| | | | | detection in various tissues |

### Example 13. Comparation and Analysis of CAR Expression Levels in T Cells and CART Cell Proliferation

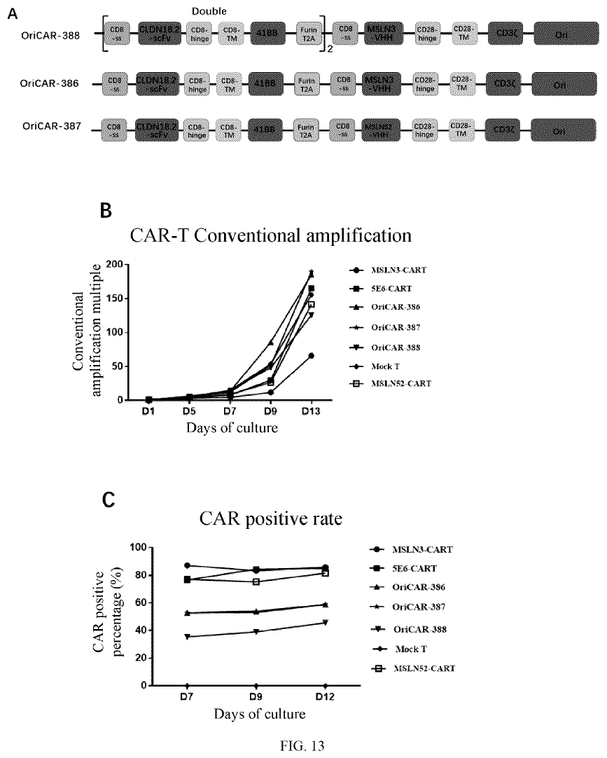

in this example, three MSLN/CLDN18.2 dual-target CARs with different structures, OriCAR-388, OriCAR-387 and OriCAR-386 were constructed at the same time. The differences in their CA R-T vector structures are shown in FIG. 13A. Compared with OriCAR-388, a double sequence was removed from OriCAR-386, i.e., a sequence of “CD8ss+CLDN18.2-scFv+CD8hinge+CD8TM-41BB+FutinT2A”, Compared with OriCAR-386, the MSLN3-VHH sequence in OriCAR-387 was replaced with another humanized sequence (named MSLN52-VHH), while other sequences and structures remain unchanged. By performing viral packing, viral titer detection, and CAR-T cell preparation using the operational methods such as those in Examples 7 and 8, 5E6-CART (CLDN18.2), MSLN3-CART, MSLNS2-CART, OriCAR-388, OriCAR-387, OriCAR-386 and Mock T cells were prepared. The cells were counted and statistically analyzed on D1/D5/D7/D9/D13 of the CAR-T cell culture. The results are shown in FIG. 13B. On the 13^{th} day of the CAR-T cell culture, the cumulative amplification multiple was approximately 125 in the OriCAR-388 T cells, approximately 189 in the OriCAR-387 T cells, approximately 185 in the OriCAR-386 T cells, and approximately 155 in the Mock T cells. The cumulative amplification multiple of OriCAR-387 was consistent with that of OriCAR-386, better than that of OriCAR-388, and was not significantly different from that of Mock T cells. The cells were subject to flow cytometry for their CAR positive rates on D7, D9 and D12 of the CAR-T cell culture, respectively. The detection antibody used was myc-tag (9B11) mouse mAb (Alexa Fluor 647)(Cell signaling, 2233S). The results are shown in FIG. 13C. The OriCAR-386, OriCAR-387 and OriCAR-388 can all be stably expressed in T cells. The CAR positive rate of OriCAR-388 is maintained at around 40%. The CAR positive rate of OriCAR-386 is consistent with that of OriCAR-387, and their CAR positive rates are maintained at around 55%, which is higher than that of OriCAR-388. That is, under conventional in vitro culture conditions, the conventional amplification multiple of dual-target OriCAR-386 and OriCAR-387 T cells is higher than that of OriCAR-388 T cells; the CAR positive rates of 5E6-CAR, MSLN3-CAR, MSLN52-CAR are higher than those of OriCAR-386 and OriCAR-387, and the positive rates of OriCAR-386 and OriCAR-387 is higher than that of OriCAR-388.

### Example 14 Comparison and Analysis of Directional Proliferation of CART Cells Under Repeated Stimulation of Target Cells In Vitro

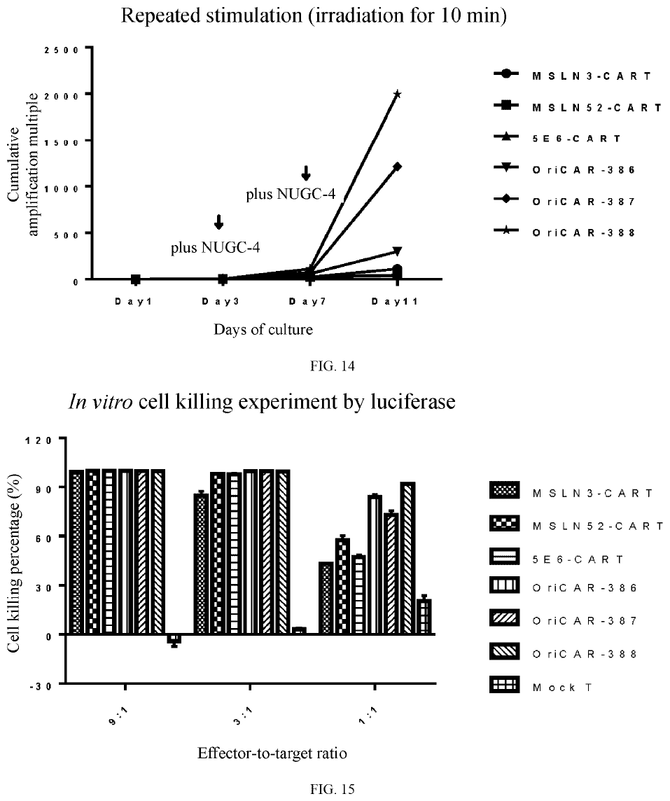

In this example, 5E6-CART, MSLN3-CART, MSLN52-CART, OriCAR-388, OriCAR-387 and OriCAR-386 cells that had been amplified and cultured in vino for 12 days in Example 13 were detected by flow cytometry for their positive rate, and then adjusted with blank T cells to allow the proportion of infected CAR-T cells be consistent and the total cell number be consistent. Specifically, the target cells were human gastric cancer cells NUGC-4 with co-expression of human CLDN18.2 and human MSLN proteins. The NUGC-4 was subject to UV irradiation for 10 min. The number of target cells was 1*10⁵/well. The effector-to-target (ET) ratio was 1:1 in the first round of stimulation, 1:2 in the second round of stimulation, and 1:2 in the third round of stimulation. Different control wells were set. The target cells and effector cells were mixed, centrifuged, re-suspended in X-VIVO blank medium, and plated into a 12-well plate. The cells were cultured in a 37° C. incubator for about 3.4 days, counted and statistically analyzed. The results are shown in FIG. 14. By comparing the dual-target CART cells, after three rounds of repeated stimulation, OriCAR-388 has the best proliferation effect, and the cumulative proliferation of T cells in the three rounds of targeted stimulation were 1996-fold after the irradiation of target cells. The second was OriCAR-387 T cells, and the cumulative proliferation of T cells in the three rounds of targeted stimulation was 1211-fold after the irradiation of target cells. OriCAR-386 T cells were relatively poor, and the cumulative proliferation of T cells in the three rounds of targeted stimulation was about 299-fold. For single-target CART cells, they can also be proliferated after three rounds of co-incubation with irradiated target cells, but the proliferation effect was very poor. The cumulative amplification of 5E6-CART cells was about 40-fold in 11 days, the cumulative amplification of MSLN3-CART cells in the three rounds of targeted stimulation was about 110-fold, and the cumulative amplification of MSLN52-CART cells in three rounds of targeted stimulation was about 45-fold. That is, under repeated stimulation of target cells in vitro, the sustained amplification capacity of OriCAR-388 T cells is stronger than that of OriCAR-387 T cells, and significantly stronger than that of OriCAR-386 T cells, while the single-target 5E6-CART, MSLN3-CART and MSLN52-CART cells only exhibit a small number of targeted amplification. This result reflects that when the CAR-T encounters a situation of large tumor loading upon injection into the body, the sustained amplification capacity of the CAR-T after repeated stimulation of the target antigen is closely related to the efficacy of CAR-T in inhibiting tumor growth, indicating that the unique CAR-T structure that OriCAR-388 T, OriCAR-387 T and OriCAR-386 T possess improves the effectiveness of sustainable amplification capacity of the CAR-T cells.

### Example 15 Comparison and Analysis of In Vitro Killing of CAR-T Cells

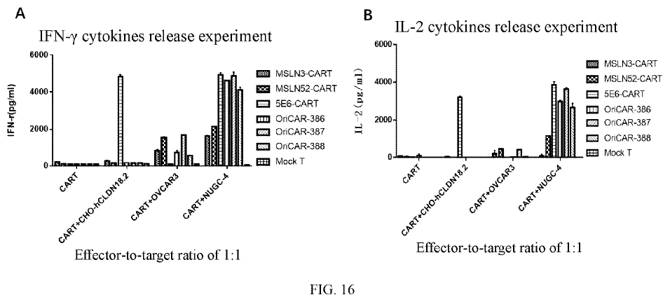

The luciferase method was used in this example to detect the killing effect of 5E6-CART, MSLN3-CART, MSLNS2-CART, OriCAR-388, OriCAR-387, OriCAR-386 and Mock T cells on NUGC4-NFkB cells in vin). CAR-T or Mock T cells that had been conventionally cultured in vitro for 11 days in the above Example 13 were selected as effector cells. The specific detection method and kit used were the same as those in Example 9. Three different effector-to-target ratios were set, i.e., E:T=1:1, 3:1, 9:1, incubation was performed for a total of 20 h before detection, and statistical analysis was performed on the detection results. The results are shown in FIG. 15. All CAR-T cells have killing activity. At the low effector-to-target ratio of 1:1, the killing activity of dual-target OriCAR-388, OriCAR-387 and OriCAR-386 T cells is significantly stronger than the killing activity of single-target 5E6-CART, MSLN3-CART and MSLN52-CART cells on target cells, among which OriCAR-388 has the strongest killing activity; under the effector-target ratio of 3:1 and 9:1, it was detected that the killing percentage of all CAR-T sequences was about 90%, but there was no difference between sequences. It shows that the unique CAR-T structure of dual-target OriCAR-388 T, OriCAR-397 T and OriCAR-386 T improves the effectiveness of CAR-T cells in specifically killing tumors.

### Example 16 Comparison and Analysis of In Vitro Cytokine Secretion by CAR-T Cells

in this example, the CAR-T or Mock T cells that had been conventionally cultured in vitro for 11 days in the above Example 13 were selected as effector cells, and CHO-hCLDN18.2, NUGC-4 and OVCAR3 cells were selected as target cells. The secretion of cytokines after co-incubation for 20 h was detected by the ELISA method as described in Example 10, and the specific detection method and kit used were the same as those in Example 10. The effector-to-target ratio was set as E:T=1:1, and the detection was performed after 20 h of co-incubation. The detection results were analyzed statistically. The results are shown in FIG. 16. When co-incubated with CHO-hCLDN18.2 cells, only E6-CART cells secreted IFN-γ and IL-2 factors; when co-incubated with OVCAR3 cells, five other CART cells, except 5E6-CART and Mock T, secreted IFN-γ and IL-2 factors at low levels, wherein the factor secretion level of MSLN52-CART cells was higher than that of the MSLN3-CART cells, and the factor secretion level of OriCAR-387 was higher than that of OriCAR-388 and OriCAR-386; when co-incubated with NUGC-4 cells, the factor secretion level of OriCAR-388, OriCAR-387 and OriCAR-386 was significantly improved, wherein OriCAR-387 has the best performance, better than the factor secretion of MSLN3-CART and MSLN52-CART cells.

### Example 17 Comparison and Analysis of Antitumor Effect of CAR-T Cells in Human Gastric Cancer Mouse CDX Tumor Models

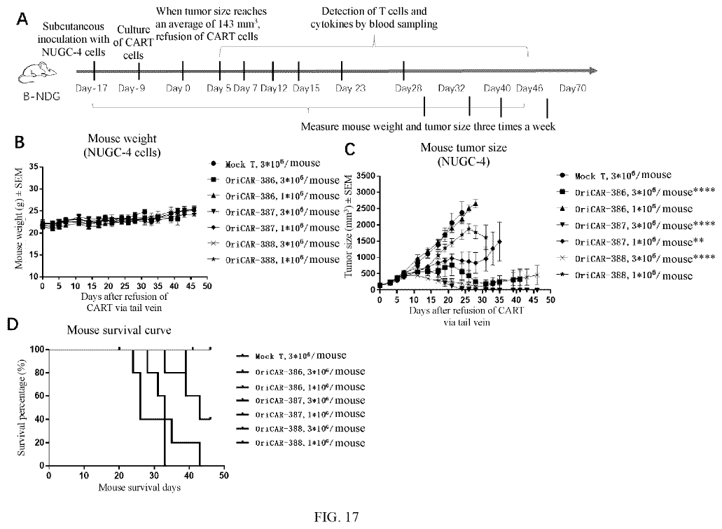

The effectiveness and safety of three different structures of MSLN/CLDN8.2 dual CAR-T cells in mice were verified in this example. The details are as follows: Target cells NUGC-4 with expression of both MSLN and CLDN18.3 antigens were cultured in vitro. Mice were subcutaneously inoculated with tumor at a dose of 3*10⁶/mouse. The tumor-bearing mice were severely immunodeficient B-NDG mice. After tumor inoculation, the mice were measured for their weight and tumor size at a frequency of three times a week. When the average tumor size of all the mice reached 143 mm³, the mice were divided into 7 groups (see FIGS. 17B, 17C and 17D for the groups), with 5 mice per group. On the day of grouping, the CART/Mock T cells were re-infused via the tail vein, and the day of refusion was recorded as PG-Day0. The mice were measured for their weight and tumor size three times a week until Day 46 after the refusion via the tail vein, i.e., PG-Day46, as shown in FIG. 17A. Throughout the observation period, except for one mouse in Group 2: OriCAR-386, 3*10⁶/mouse group, which had a weight loss rate of more than 15% and was euthanized on PG-D41, the body weight and physiology of all other mice included in the group during the experiment kept a normal status, as shown in FIG. 17B. At the end of the experiment, OriCAR-386/OriCAR-387/OriCAR-388 T cells showed extremely significant anti-tumor efficacy compared to Mock T cells in the human gastric cancer mouse transplantation models, and the anti-tumor activity of OriCAR-387 is better than that of OriCAR-388, and the anti-tumor activity of OriCAR-388 is better than that of OriCAR-386, as shown in FIG. 17C (the endpoint is defined as mouse tumor size >3000 mm³): mice in the OriCAR-386/OriCAR-387/OriCAR-388 T cell high-dose group (3*10⁶/mouse) all have extremely significant antitumor activity compared to mice in the Mock T cell group (3*10⁶/mouse), with a statistical difference of P<0.0001. Among them, three mice in the OriCAR-386 T cell high-dose group achieved tumor elimination (3.1), and five mice in the OriCAR-387 T cell high-dose group achieved tumor elimination (5/5), two mice in the OriCAR-388 T cell high-dose group achieved tumor elimination (2/5); compared with the Mock T cell group, the OriCAR-386 low-dose group (1*10⁶/mouse) showed no significant anti-tumor activity, there was no statistical difference. The OriCAR-387 low-dose group (1*10⁶/mouse) showed significant anti-tumor activity (P<0.01), and the OriCAR-388 low-dose group (1*10⁶/mouse) showed no significant anti-tumor activity, and there was no statistical difference. Among them, no mice in the OriCAR-386 low-dose group had tumors eliminated (0/5), two mice in the OriCAR-387 low-dose group had tumors eliminated (2/5), and one mouse in the OriCAR-388 low-dose group had tumors eliminated (1/5). The mouse survival curve is shown in FIG. 17D. By the end of the experiment (tumor size >3000 mm³), mice in the OriCAR-386/OriCAR-387/OriCAR-388 T cell high-dose group (3*100/mouse) could achieve 100% survival rate. The statistical results showed that the median survival period of mice in the Mock T (1*10⁶/mouse) group was 26 days, the median survival period of mice in the OriCAR-386 (1*10⁶/mouse) group was 33 days, the median survival period of mice in the OriCAR-387 (1*10⁶/mouse) group was 43 days, and the median survival period of mice in the OriCAR-388 (1*10⁶/mouse) group was 43 days. In general, in the human gastric cancer mouse CDX tumor models, the safety and antitumor efficacy of OriCAR-387 T cells were better than those of OriCAR-388 T cells and OriCAR-386 T cells.

### Example 18 Measurement of Specific Binding Activity of CLDN18.2 Targeting Moiety to CLDN18.2

By fluorescence-activated cell sorting (FACS), the specific binding activity of the chimeric antibody to the target cells was detected by iQue Screener flow cytometer (purchased from intelliCyt Company) using PBS containing 0.1% BSA as buffer. Three target cells including a stably transfected cell line expressing human CLDN18.2, a stably transfected cell line expressing human CLDN18.1, and a tumor cell line were selected for the detection of the binding activity, respectively.

1. Detection of Binding Activity of the CLDN18.2 Antigen-Binding Protein of the Present Application to Cells with High Expression of Human CLDN18.2 by Flow Cytometry

Cell lines with stably high expression of CLDN18 were constructed and labeled as 293T-human CLDN18.2. CHO-human CLDN18.2 and SP2/0-human CLDN18.2 cells respectively. The cells were digested, counted, re-suspended in a flow buffer, adjusted to 1×10⁶/ml, and added to a V-bottomed 96-well plate at 30 μl/well. A primary antibody was added at 30 μl/well, and subject to 2- or 3-fold gradient dilution with the flow buffer to form 7 gradients starting from a concentration of 30 μg/ml. A PBS negative control was set for each antibody. The positive control antibody was Zolbetuximab purified in Example 1. The plate was incubated at 4° C. for 1 h, and washed once with the flow buffer. A secondary antibody (abcam, Cat #ab98593) was added at 30 μl/well, incubated at 4° C. for 30 min, and washed twice with the flow buffer. The cells were shaken to loose, and the flow buffer was added at 25 μl/well for loading. The original data were introduced into the GraphPad8.0 software for plotting and calculation. The results are shown in FIG. 18.

2. Detection of Binding Activity of the CLDN18.2 Antigen-Binding Protein of the Present Application to Cells with High Expression of Human CLDN18.1 by Flow Cytometry

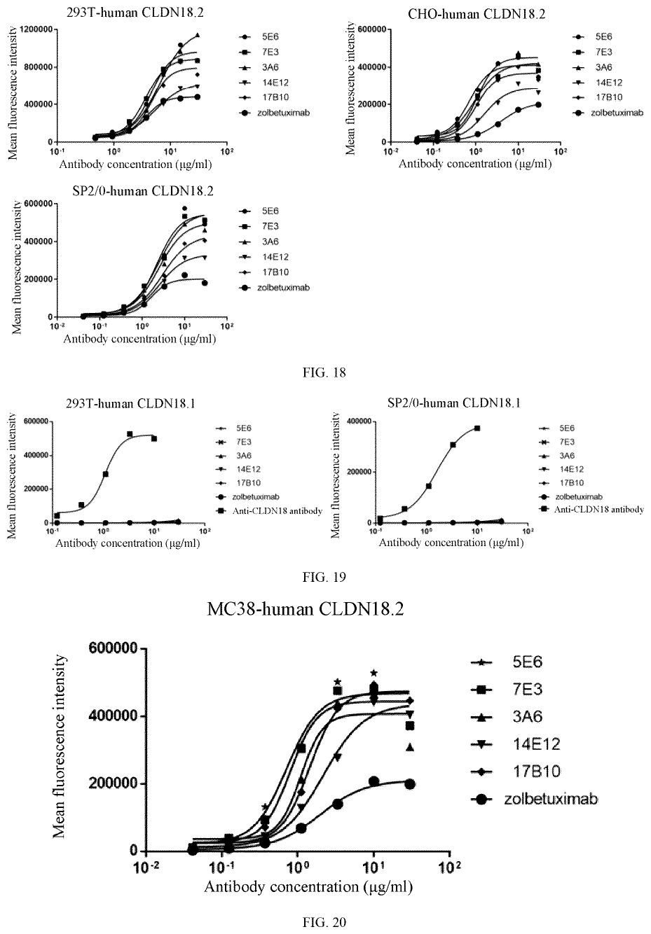

The positive control antibody was a commercially available anti-CLDN18 antibody (Anti-Claudin18 antibody) (abcam, Cat #ab203563), which had an antigen-binding site located in the intracellular part of the CLDN18.2 quaternary transmembrane protein, and required flow cytometric intracellular staining analysis. Specifically, the cells were the constructed 293T-human CLDN18.1 and SP2/0-human CLDN18.1 cells. The cells were digested, counted, fixed, and subject to rupture treatment of cell membrane. The treated cells were re-suspended in a flow buffer to 1×10⁶/ml, and added to a V-bottomed 96-well plate at 30 μl/well. A primary antibody was added at 30 μl/well, and subject to 3-fold gradient dilution with the flow buffer to form 7 gradients starting from a concentration of 30 μg/ml A PBS negative control was set for each antibody. The positive control antibody was diluted under the same conditions as above. The plate was incubated at 4° C. for 1 h, and washed once with the flow buffer. A secondary antibody (Cat #ab98593 and Cat #ab150079) was added at 30 μl/well, incubated at 4° C. for 30 min; and washed twice with the flow buffer. The cells were shaken to loose, and the flow buffer was added at 25 μl/well for loading. The original data were introduced into the GraphPad8.0 software for plotting and calculation. The results are shown in FIG. 19.

### 3. Detection of Binding Activity of the CLDN18.2 Antigen-Binding Protein of the Present Application to the Tumor Cell Lines by Flow Cytometry

The constructed tumor cells MC38-human CLDN18.2 with stably high expression of human CLDN18.2 were selected as the target cells. The cells were digested, counted, re-suspended in a flow buffer, adjusted to 1×10⁶/ml, and added to a V-bottomed 96-well plate at 30 μl/well. A primary antibody was added at 30 μl/well, and subject to 3-fold gradient dilution with the flow buffer to form 7 gradients starting from a concentration of 30 μg/ml. A PBS negative control was set for each antibody. The positive control antibody was the Zolbetuximab obtained by purification, which was diluted under the same conditions as above. The plate was incubated at 4° C. for 1 h, and washed once with the flow buffer. A secondary antibody (abcam, Cat #ab98593) was added at 30 μl/well, incubated at 4° C. for 30 min, and washed twice with the flow buffer. The cells were shaken to loose, and the flow buffer was added at 25 μl/well for loading. The original data were introduced into the GraphPad8.0 software for plotting and calculation. The results are shown in FIG. 20.

By the above operation steps, five CLDN18.2 antigen-binding proteins were obtained by expression and purification (wherein, the 5E6 has a VH sequence as set forth in SEQ ID NO: 26, and a VL sequence as set forth in SEQ ID NO:34; the 7E3 has a VH sequence as set forth in SEQ ID NO: 73, and a VL sequence as set forth in SEQ ID NO: 49; the 3A6 has a VH sequence as set forth in SEQ ID NO: 61, and a VL sequence as set forth in SEQ ID NO: 65; the 14E12 has a VH sequence as set forth in SEQ ID NO: 79, and a VL sequence as set forth in SEQ ID NO: 87, the 17B10 has a VH sequence as set forth in SEQ ID NO: 79, and a VL sequence as set forth in SEQ ID NO: 93), which were verified by flow cytometry for their antigen-binding activity. As shown in FIG. 18, the five antigen-binding proteins all show concentration-dependent binding activity to human CLDN18.2, and most of them are stronger than the Zolbetuximab positive antibody, and the test results are consistent for the three types of cells. As shown in FIG. 19, all the five antigen-binding proteins can specifically bind to human CLDN18.2, but not bind to human CLDN18.1. As shown in FIG. 20, all the five antigen-binding proteins strongly bind to the mouse colon cancer MC38 cells with stably high expression of human CLDN18.2, and the binding activity intensity is concentration-dependent, with the effect equivalent to or stronger than that of the Zolbetuximab positive antibody (with a heavy chain sequence as set forth in SEQ ID NO: 157 and a light chain sequence as set forth in SEQ ID NO: 158).

The foregoing detailed description is provided by way of explanation and examples and is not intended to limit the scope of the appended claims. Various changes of the embodiments currently set forth in this application are obvious to those of ordinary skills in the art and are reserved within the scope of the appended claims and their equivalents.

| ID | LABEL | NOTE | TYP | ORG | SEQ | ALIGN |
|---|---|---|---|---|---|---|
| 1 | Human | Human | DNA | Artificial | atggctctgcctgtgaccgctctgctgctg | |
| | CD8 | CD8 | | sequence | cctctggctctgctgctgcatgccgcaaga | |
| | signal | signal | | | cct | |
| | peptide | peptide | | | | |
| | (DNA) | (DNA) | | | | |
| 2 | Human | Human | PRT | Artificial | MALPVTALLLPLALLLHAARP | |
| | CD8 | CD8 | | sequence | | |
| | signal | signal | | | | |
| | peptide | peptide | | | | |
| | (PRT) | (PRT) | | | | |
| 3 | Human | Human | DNA | Artificial | accacgacgccagcgccgcggccgccaaca | |
| | CD8 | CD8 | | sequence | ccggcgcccaccatcgcgtcgcagcccctg | |
| | hinge | hinge | | | tccctgcgcccagaggcgtgccggccagcg | |
| | region | region | | | gggggggcgcagtgcacacgagggggctgg | |
| | (DNA) | (DNA) | | | acttcgcctgtgat | |
| 4 | Human | Human | PRT | Artificial | TTTPAPRPPTPAPTIASQPLSERPEACRPA | |
| | CD8 | CD8 | | sequence | AGGAVHTRGLDFACD | |
| | hinge | hinge | | | | |
| | region | region | | | | |
| | (PRT) | (PRT) | | | | |
| 5 | Human | Human | DNA | Artificial | atctacatctgggcgcccttggccgggact | |
| | CD8 | CD8 | | sequence | tgtggggtccttctcctgtcactggttatc | |
| | trans- | trans- | | | accctttactgc | |
| | membrane | membrane | | | | |
| | region | region | | | | |
| | (DNA) | (DNA) | | | | |
| 6 | Human | Human | PRT | Artificial | IYIWAPLAGTCGVLLLSLVITLYC | |
| | CD8 | trans- | | sequence | | |
| | CD8 | membrane | | | | |
| | trans- | region | | | | |
| | membrane | (PRT) | | | | |
| | region | | | | | |
| | (PRT) | | | | | |
| 7 | Human | Human | DNA | Artificial | aaacggggcagaaagaaactcctgtatata | |
| | 41BB | 41BB | | sequence | ttcaaacaaccatttatgagaccagtacaa | |
| | intra- | intra- | | | actactcaagaggaagatggctgtagctgc | |
| | cellular | cellular | | | cgatttccagaagaagaagaaggaggatgt | |
| | region | region | | | gaactg | |
| | (DNA) | (DNA) | | | | |
| 8 | Human | Human | PRT | Artificial | KRGRKKLLYIFKQPFMRPVQTTQEEDGCSC | |
| | 41BB | 41BB | | sequence | REPEEEEGGCEL | |
| | intra- | intra- | | | | |
| | cellular | cellular | | | | |
| | region | region | | | | |
| | (PRT) | (PRT) | | | | |
| 9 | FurinT2 | FurinT2 | DNA | Artificial | cgtagaaagcgttcgggatcgggagagggc | |
| | A | A | | sequence | agaggaagtcttctaacatgcggtgacgtg | |
| | self- | self- | | | gaggagaatcccggccct | |
| | cleaving | cleaving | | | | |
| | peptide | peptide | | | | |
| | (DNA) | (DNA) | | | | |
| 10 | FurinT2 | FurinT2 | PRT | Artificial | RRKRSGSGEGRGSLLTCGDVEENPGP | |
| | A | A | | sequence | | |
| | self- | self- | | | | |
| | cleaving | cleaving | | | | |
| | peptide | peptide | | | | |
| | (PRT) | (PRT) | | | | |
| 11 | Human | Human | DNA | Artificial | atcgaggtgatgtacccccctccctacctg | |
| | CD28 | CD28 | | sequence | gacaacgagaagagcaacggcaccatcatc | |
| | hinge | hinge | | | cacgtgaagggcaagcacctgtgccctagc | |
| | region | region | | | cccctgttccccggcccct | |
| | (DNA) | (DNA) | | | | |
| 12 | Human | Human | PRT | Artificial | TEVMYPPPYLDNEKSNGTIIHVKGKHLCPS | |
| | CD28 | CD28 | | sequence | PLFPGPSKP | |
| | hinge, | hinge | | | | |
| | region | region | | | | |
| | (PRT) | (PRT) | | | | |
| 13 | Human | Human | DNA | Artificial | ttctgggtgctcgtcgtgggggcggcgtgc | |
| | CD28 | CD28 | | sequence | tggcctgctacagcctgctggtgaccgtgg | |
| | trans- | trans- | | | cattcatcatcttctgcgtg | |
| | membrane | membrane | | | | |
| | region | region | | | | |
| | (DNA) | (DNA) | | | | |
| 14 | Human | Human | PRT | Artificial | FWVLVVVGGVLACYSLLVTVAFIIFWV | |
| | CD28 | CD28 | | sequence | | |
| | trans- | trans- | | | | |
| | membrane | membrane | | | | |
| | region | region | | | | |
| | (PRT) | (PRT) | | | | |
| 15 | Human | Human | DNA | Artificial | agagtgaagttcagcaggagcgcagacgcc | |
| | CD3ζ | CD3ζ | | sequence | cccgcgtaccagcagggccagaaccagctc | |
| | intra- | intra- | | | tataacgagctcaatctaggacgaagagag | |
| | cellular | cellular | | | gagtacgatgtattggacaagagacgtggc | |
| | region | region | | | cgggaccctgagatggggggaaagccgaga | |
| | (DNA) | (DNA) | | | aggaaganccctcaggaaggcctgtacaat | |
| | | | | | gaactgcagaaagataagatggcggaggcc | |
| | | | | | tacagtgagattgggatgaaaaggcgagcg | |
| | | | | | ccggaggggcaaggggcacgatggccttta | |
| | | | | | ccagggtctcagtacagccaccaaggacac | |
| | | | | | ctacgacgcccttcacatgcaggccctgcc | |
| | | | | | ccctcgc | |
| 16 | Human | Human | PRT | Artificial | RVKFSRSADAPAYQQGQNQLYNELNLGRRE | |
| | CD3ζ | CD3ζ | | sequence | EYDVLDKRRGRDPEMGGKPRRKNPQEGLYN | |
| | intra- | intra- | | | ELQKDKMAEAYSEIGMKGERRRGKGHDGLY | |
| | cellular | cellular | | | QGLSTATKDTYDALHMQALPPR | |
| | region | region | | | | |
| | (PRT) | (PRT) | | | | |
| 17 | Ori | Ori | DNA | Artificial | gagggcagaggaagtcttctaacatgcggt | |
| | element | element | | sequence | gacgtggaggagaatcccggccctatgggg | |
| | (DNA) | (DNA) | | | gccgtcctgaggagcctcctggcctgcagc | |
| | | | | | ttctgtgtgctcctgagagcggagcagaaa | |
| | | | | | ctcatctctgaagaggatctggaacctcca | |
| | | | | | acatgttctcctcagcagtttacttgtttc | |
| | | | | | acgggggaaattgactgtatccctgtggct | |
| | | | | | tggcggtgcgatgggtttactgaatgtgaa | |
| | | | | | gaccacagtgatgaactcaattgtcctgta | |
| | | | | | tgctcagagtcccagttccagtgtgccagt | |
| | | | | | gggcagtgtattgatggtgccctccgatgc | |
| | | | | | aatggagatgcaaactgccaggacaaatca | |
| | | | | | gatgagaagaactgtgaagtgctttgttta | |
| | | | | | attgatcagttccgctgtgccaatggtcag | |
| | | | | | tgcattggaaagcacaagaagtgtgatcat | |
| | | | | | aatgtggattgcagtgacaagtcagatgaa | |
| | | | | | ctggattgttatccgactgaagaaccagca | |
| | | | | | ccacaggccaccaatacagttggttctgtt | |
| | | | | | attggcgtaattgtcaccatttttgtgtct | |
| | | | | | ggaactgtatactttatctgccagaggatg | |
| | | | | | ttgtgtccacgtatgaagggagatggggaa | |
| | | | | | actatgactaatgactatgtagttcatgga | |
| | | | | | ccagcttctgtgcctcttggttatgtgcca | |
| | | | | | cacccaagttctttgtcaggatctcttcca | |
| | | | | | ggaatgtctcgaggtaaatcaatgatcagc | |
| | | | | | tccctcagtatcatggggggaagcagtgga | |
| | | | | | cccccctatgaccgagcccatgttacagga | |
| | | | | | gcatcatcaagtagttcttcaagcaccaaa | |
| | | | | | ggcacttacttccctgcaattttgaaccct | |
| | | | | | ccaccatccccagccacagagcgatcacat | |
| | | | | | tacactatggaattggatattcttcaaaca | |
| | | | | | gtccttccactcataggtcatacagctaca | |
| | | | | | ggccatatagctaccggcactttgcacccc | |
| | | | | | ccaccacaccctgcagcacagatgtttgtg | |
| | | | | | acagtgactatgctcctagtcggagaatga | |
| | | | | | cctcagtggcaacagccaagggctatacca | |
| | | | | | gtgacttgaactatgattcagaacctgtgc | |
| | | | | | ccccacctcccacaccccgaagccaatact | |
| | | | | | tgtcagcaagaactatgaaagctgcccacc | |
| | | | | | ttctccatacacagagaggagctattctca | |
| | | | | | tcacctctacccaccgccaccctctccctg | |
| | | | | | tacagactcctcc | |
| 18 | Ori | Or | PRT | Artificial | EGRGSLLTCGDVEENPGPMGAVLRSLLACS | |
| | element | element | | sequence | FCVLLRAEQKLISEEDLEPPTCSPQQFTCF | |
| | (PRT) | (PRT) | | | TGEIDCIPVAWRCDGFTECEDHSDELNCPV | |
| | | | | | CSESQFQCASGQCIDGALRQNGDANCQDKS | |
| | | | | | DEKNCEVLCLIDQFRCANGQCIGKHKKCDH | |
| | | | | | NVDCSDKSDELDCYPTEEPAPQATNTVGSV | |
| | | | | | IGVIVTIFVSGTVYFICQRMLCPRMKGDGE | |
| | | | | | TMTNDYVVHGPASVPLGYVPHPSSLSGSLP | |
| | | | | | GMSRGKSMISSLSIMGGSSGPPYDRAHVTG | |
| | | | | | ASSSSSSSTKGTYFPAILNPPPSPATERSH | |
| | | | | | YTMEFGYSSNSPSTHRSYSYRPYSYRHFAP | |
| | | | | | PTTPCSTDVCDSDYAPSRRMTSVATAKGYT | |
| | | | | | SDLNYDSEPVPPPPTPRSQYLSAEENYESC | |
| | | | | | PPSPYTERSYSHHLYPPPPSPCTDSS | |
| 19 | 5E6 | HCDR1 | DNA | Artificial | AACTATGTGATGAAC | |
| | HCDR1 | | | sequence | | |
| | (DNA) | | | | | |
| 20 | 5E6 | HCDR1 | PRT | Artificial | NYVMN | |
| | HCDR1 | | | sequence | | |
| 21 | 5E6 | HCDR2 | DNA | Artificial | TATATTAACCCATATAATGATGGCACCAAA | |
| | HCDR2 | | | sequence | TATAATGAAAGATITAAAGGC | |
| | (DNA) | | | | | |
| 22 | 5E6 | HCDR2 | PRT | Artificial | YINPYNDGTKYNERFKG | |
| | HCDR2 | | | sequence | | |
| 23 | 5E6 | HCDR3 | DNA | Artificial | CTGTATAGAGGCAATGCCATGGATTAT | |
| | HCDR3 | | | sequence | | |
| | (DNA) | | | | | |
| 24 | 5E6 | HCDR3 | PRT | Artificial | LYRGNAMDY | |
| | HCDR3 | | | sequence | | |
| 25 | 5E6 | VH | DNA | Artificial | CAAGTGCAGCTGGTGCAGAGTGGTGCAGAA | |
| | VH | | | sequence | GTGAAAAAACCTGGTGCAAGTGTGAAAGTG | |
| | (DNA) | | | | AGCTGCANAGCAAGTGGCTATACCTTTACC | |
| | | | | | AACTATGTGATGAACTGGGTGAGACAAGCC | |
| | | | | | CCTGGTCAGAGACTGGAATGGATGGGCTAT | |
| | | | | | ATTAACCCATATAATGATGGCACCAAATAT | |
| | | | | | AATGAAAGATTTAAAGGCAGAGTCACCATC | |
| | | | | | ACTAGAGATACAAGTGCAAGCACTGCCTAT | |
| | | | | | ATGGAACTGAGCAGCCTGAGAAGTGAAGAT | |
| | | | | | ACTGCAGTGTATTATTGTGCAAGACTGTAT | |
| | | | | | AGAGGCAATGCCATGGATTATTGGGGCCAA | |
| | | | | | GGCACCCTGGTGACTGTGAGCAGC | |
| 26 | 5E6 | VH | PRT | Artificial | QVQLVQSGAEVKKPGASVKVSCKASGYTFT | |
| | VH | | | sequence | NYVMNWVRQAPGQRLEWMGYINPYNDGTKY | |
| | | | | | NERFKGRVTITRDTSASTAYMELSSLRSED | |
| | | | | | TAVYYCARLYRGNAMDYWGQGTLVTVSS | |
| 27 | 5E6 | LCDR1 | DNA | Artificial | AAAAGCAGTCAGAGCCTGCTGGCCAGTGGC | |
| | LCDR1 | | | sequence | AATCAGAAAAACTATCTGACC | |
| | (DNA) | | | | | |
| 28 | 5E6 | LCDR1 | PRT | Artificial | KSSQSLLASGNQKNYLT | |
| | LCDR1 | | | sequence | | |
| 29 | 5E6 | LCDR2 | DNA | Artificial | TGGGCAAGCACTAGAGAAAGT | |
| | LCDR2 | | | sequence | | |
| | (DNA) | | | | | |
| 30 | 5E6 | LCDR2 | PRT | Artificial | WASTRES | |
| | LCDR2 | | | sequence | | |
| 31 | 5E6 | LCDR3 | DNA | Artificial | CAGAATGTGTATATTTATCCATTTACC | |
| | LCDR3 | | | sequence | | |
| | (DNA) | | | | | |
| 32 | 5E6 | LCDR3 | PRT | Artificial | QNVYTYPET | |
| | LCDR3 | | | sequence | | |
| 33 | 5E6 | VL | DNA | Artificial | GATATTGTGATGACTCAGAGCCCTGATAGC | |
| | VL | | | sequence | CTGGCAGTGAGCCTGGGTGAAAGAGCCACC | |
| | (DNA) | | | | ATTAACTGCAAAAGCAGTCAGAGCCTGCTG | |
| | | | | | GCCAGTGGCAATCAGAAAAACTATCTGACC | |
| | | | | | TGGTATCAGCAGAAACCTGGTCAGCCACCA | |
| | | | | | AAACTGCTGATTTATTGGGCAAGCACTAGA | |
| | | | | | GAAAGTGGTGTGCCTGATAGATTTAGTGGC | |
| | | | | | AGTGGCAGTGGCACTGATTITACCCTGACC | |
| | | | | | ATTAGCAGCCTGCAAGCAGAAGATGTGGCA | |
| | | | | | GTGTATTATTGTCAGAATGTGTATATTTAT | |
| | | | | | CCATTTACCTTTGGCCAAGGCACCAAACTG | |
| | | | | | GAAATTAAAAGA | |
| 34 | 5E6 VL | VL | PRT | Artificial | DIVMTQSPDSLAVSLGERATINCKSSQSLL | |
| | | | | sequence | ASGNQKNYLTWYQQKPGQPPKLLIYWASTR | |
| | | | | | ESGVPDRFSGSGSGTDFTLTISSLQAEDVA | |
| | | | | | VYYCQNVYTYPFTFGQGTKLEIKR | |
| 35 | Linker of | Linker | DNA | Artificial | GCCTCCACCGGTGGGGGTGGAAGCGGCGGT | |
| | 5E6 scFv | | | sequence | GGCGGAAGCGGCGGTGGCGGCAGC | |
| | (DNA) | | | | | |
| 36 | Linker of | Linker | PRT | Artificial | ASTGGGGSGGGGSGGGGS | |
| | 5E6 scFv | | | sequence | | |
| 37 | 5E6 scFv | scFv | DNA | Artificial | CAAGTGCAGCTGGTGCAGAGTGGTGCAGAA | |
| | (DNA) | | | sequence | GTGAAAAAACCTGQTGCAAGTGTGAAAGTG | |
| | | | | | AGCTGCAAAGCAAGTGGCTATACCTTTACC | |
| | | | | | AACTATGTGATGAACTGGGTGAGACAAGCC | |
| | | | | | CCTGGTCAGAGACTGGAATGGATGGGCTAT | |
| | | | | | ATTAACCCATATAATGATGGCACCAAATAT | |
| | | | | | AATGAAAGATTTAAAGGCAGAGTCACCATC | |
| | | | | | ACTAGAGATACAAGTGCAAGCACTGCCTAT | |
| | | | | | ATGGAACTGAGCAGCCTGAGAAGTGAAGAT | |
| | | | | | ACTGCAGTGTATTATTGTGCAAGACTGTAT | |
| | | | | | AGAGGCAATGCCATGGATTATTGGGGCCAA | |
| | | | | | GGCACCCTGGTGACTGTGAGCAGCGCCTCC | |
| | | | | | ACCGGTGGCGGTGGAAGCGGCGGTGGCGGA | |
| | | | | | AGCGGCGGTGGCGGCAGCGATATTGTGATG | |
| | | | | | ACTCAGAGCCCTGATAGCCTGGCAGTGAGC | |
| | | | | | CTGGGTGAAAGAGCCACCATTAACTGCAAA | |
| | | | | | AGCAGTCAGAGCCTGCTGGCCAGTGGCAAT | |
| | | | | | CAGAAAAACTATCTGACCTGGTATCAGCAG | |
| | | | | | AAACCTGGTCAGCCACCAAAACTGCTGATT | |
| | | | | | TATTGGGCAAGCACTAGAGAAAGTGGTGTG | |
| | | | | | CCTGATAGATTTAGTGGCAGTGGCAGTGGC | |
| | | | | | ACTGATTTTACCCTGACCATTAGCAGCCTG | |
| | | | | | CAAGCAGAAGATGTGGCAGTGTATTATTGT | |
| | | | | | CAGAATGTGTATATTTATCCATTTACCTTT | |
| | | | | | GGCCAAGGCACCAAACTGGAAATTAAAAGA | |
| 38 | 5E6 | scFv | PRT | Artificial | QVQLVQSGAEVKKPGASVKVSCKASGYTFT | |
| | scFv | | | sequence | NYVMNWVRQAPGQRLEWMGYINPYNDGTKY | |
| | | | | | NERFKGRVTITRDTSASTAYMELSSLRSED | |
| | | | | | TAVYYCARLYRGNAMDYWGQGTLVTVSSAS | |
| | | | | | TGGGGSGGGGSGGGGSDIVMTQSPDSLAVS | |
| | | | | | LGERATINCKSSQSLLASGNQKNYLTWYQQ | |
| | | | | | KPGQPPKLLIYWASTRESGVPDRFSGSGSG | |
| | | | | | TDETLTISSLQAEDVAVYYCQNVYIYPFTF | |
| | | | | | GQGTKLEIKR | |
| 39 | 5E5 | HCDR1 | DNA | Artificial | AACTATGTTATGAAC | |
| | HCDR1 (DNA) | | | sequence | | |
| 20 | 5E5 HCDR1 | HCDR1 | PRT | Artificial | NYVMN | |
| | | | | sequence | | |
| 40 | 5E5 HCDR2 | HCDR2 | DNA | Artificial | TATATTAATCCTTACAATGATGGTACTAAG | |
| | (DNA) | | | sequence | TACAATGAGAGGTTCAAAGGC | |
| 22 | 5E5 HCDR2 | HCDR2 | PRT | Artificial | YINPYNDGTKYNERFKG | |
| | | | | sequence | | |
| 41 | 5E5 HCDR3 | HCDR3 | DNA | Artificial | CTATATAGAGGCAATGCTATGGACTAC | |
| | (DNA) | | | sequence | | |
| 24 | 5E5 HCDR3 | HCDR3 | PRT | Artificial | LYRGNAMDY | |
| | | | | sequence | | |
| 42 | 5E5 VH (DNA) | VH | DNA | Artificial | GAGGTCCAGCTGCAGCAGTCTGGACCTGAG | |
| | | | | sequence | CTGGTAAAGCCTGGGGCTTCAGTGAAGATG | |
| | | | | | TCCTGCAAGGCTTCTGGATACACATTCACT | |
| | | | | | AACTATGTTATGAACTGGGTGAAGCAGAAG | |
| | | | | | CCTGGGCAGGGCCTTGAGTGGATTGGATAT | |
| | | | | | ATTAATCCTTACAATGATGGTACTAAGTAC | |
| | | | | | AATGAGAGGTTCAAAGGCAAGGCCACACTG | |
| | | | | | ACTTCAGACAAATCCTCCAGCACAGCCTTC | |
| | | | | | ATGGAGGTCAGCAGCCTGACCTCTGAGGAC | |
| | | | | | TCTGCGGTCTACTACTGTGCAAGACTATAT | |
| | | | | | AGAGGCAATGCTATGGACTACTGGGGTCAA | |
| | | | | | GGAACCTCGGTCACCGTCTCCTCA | |
| 43 | 5E5 VH | VH | PRT | Artificial | EVQLQQSGPELVKPGASVKMSCKASGYTET | |
| | | | | sequence | NYVMNWVKQKPGQGLEWIGYINPYNDGTKY | |
| | | | | | NERFKGKATLTSDKSSSTAFMEVSSLTSED | |
| | | | | | SAVYYCARLYRGNAMDYWGQGTSVTVSS | |
| 44 | 5E5 LCDR1 | LCDR1 | DNA | Artificial | AAGTCCAGTCAGAGTCTGTTAAACAGTGGA | |
| | (DNA) | | | sequence | AATCAAAAGAACTACTTGACC | |
| 45 | 5E5 LCDR1 | LCDR1 | PRT | Artificial | KSSQSLLNSGNQKNYLT | |
| | | | | sequence | | |
| 46 | 5E5 LCDR2 | LCDR2 | DNA | Artificial | TGGGCATCCACTAGGGAATCT | |
| | (DNA) | | | sequence | | |
| 30 | 5E5 LCDR2 | LCDR2 | PRT | Artificial | WASTRES | |
| | | | | sequence | | |
| 47 | 5E5 LCDR3 | LCDR3 | DNA | Artificial | CAGAATGTTTATATTTATCCGTTCACG | |
| | (DNA) | | | sequence | | |
| 32 | 5E5 LCDR3 | LCDR3 | PRT | Artificial | QNVYIYPFT | |
| | | | | sequence | | |
| 48 | 5E5VL (DNA) | VL | DNA | Artificial | GACATTGTGATGACACAGTCTCCATCCTCC | |
| | | | | sequence | CTGACTGTGACAGTAAGAGAGAAGGTCACT | |
| | | | | | TTGAGCTGCAAGTCCAGTCAGAGTCTGTTA | |
| | | | | | AACAGTGGAAATCAAAAGAACTACTTGACC | |
| | | | | | TGGTACCAGCAGAAACCAGGGCAGCCTCCT | |
| | | | | | AAACTTTTGATCTACTGGGCATCCACTAGG | |
| | | | | | GAATCTGGGGTCCCTGATCGCTTCACAGGC | |
| | | | | | AGTGGATCTGGCACAGATTTCACTCTCACC | |
| | | | | | ATCAGCAGTGTGCAGGCTGAAGACCTGGCA | |
| | | | | | GTTTATTACTGTCAGAATGTTTATATTTTA | |
| | | | | | TCCGTTCACGTTCGGTGCTGGGACCAAGCT | |
| | | | | | GGAGCTGAAACGG | |
| 49 | 5E5VL | VL | PRT | Artificial | DIVMTQSPSSLTVTVREKVTLSCKSSQSLL | |
| | | | | sequence | NSGNQKNYLTWYQQKPGQPPKLLIYWASTR | |
| | | | | | ESGVPDRFTGSGSGTDFTLTISSVQAEDLA | |
| | | | | | VYYCQNVYTYPFTFGAGTKLELKR | |
| 50 | Linker of | Linker | DNA | Artificial | GGCGGAGGAGGATCCGGAGGCGGAGGAAGC | |
| | 5E5 scFv (DNA) | | | sequence | GGAGGAGGCGGATCT | |
| 51 | Linker of | Linker | PRT | Artificial | GGGGSGGGGSGGGGS | |
| | 5E5 scFv | | | sequence | | |
| 52 | 5E5 scFv (DNA) | scFv | DNA | Artificial | GACATTGTGATGACACAGTCTCCATCCTCC | |
| | | | | sequence | CTGACTGTGACAGTAAGAGAGAAGGTCACT | |
| | | | | | TTGAGCTGCAAGTCCAGTCAGAGTCTGTTA | |
| | | | | | AACAGTGGAAATCAAAAGAACTACTTGACC | |
| | | | | | TGGTACCAGCAGAAACCAGGGCAGCCTCCT | |
| | | | | | AAACTTTTGATCTACTGGGCATCCACTAGG | |
| | | | | | GAATCTGGGGTCCCTGATCGCTTCACAGGC | |
| | | | | | AGTGGATCTGGCACAGATTTCACTCTCACC | |
| | | | | | ATCAGCAGTGTGCAGGCTGAAGACCTGGCA | |
| | | | | | GTTTATTACTGTCAGAATGTTTATATTTAT | |
| | | | | | CCGTTCACGTTCGGTGCTGGGACCAAGCTG | |
| | | | | | GAGCTGAAACGGGGCGGAGGAGGATCCGGA | |
| | | | | | GGCGGAGGAAGCGGAGGAGGCGGATCTGAG | |
| | | | | | GTCCAGCTGCAGCAGTCTGGACCTGAGCTG | |
| | | | | | GTAAAGCCTGGGGCTTCAGTGAAGATGTCC | |
| | | | | | TGCAAGGCTTCTGGATACACATTCACTAAC | |
| | | | | | TATGTTATGAACTGGGTGAAGCAGAAGCCT | |
| | | | | | GGGCAGGGCCTTGAGTGGATTGGATATATT | |
| | | | | | AATCCTTACAATGATGGTACTAAGTACAAT | |
| | | | | | GAGAGGTTCAAAGGCAAGGCCACACTGACT | |
| | | | | | TCAGACAAATCCTCCAGCACAGCCTTCATG | |
| | | | | | GAGGTCAGCAGCCTGACCTCTGAGGACTCT | |
| | | | | | GCGGTCTACTACTGTGCAAGACTATATAGA | |
| | | | | | GGCAATGCTATGGACTACTGGGGTCAAGGA | |
| | | | | | ACCTCGGTCACCGTCTCCTCA | |
| 53 | 5E5 scFv | scFv | PRT | Artificial | DIVMTQSPSSLTVTVREKVTLSCKSSQSLL | |
| | | | | sequence | NSGNQKNYLTWYQQKPGQPPKLLIYWASTR | |
| | | | | | ESGVPDRFTGSGSGTDFTLTISSVQAEDLA | |
| | | | | | VYYCQNVYIYPFTFGAGTKLELKRGGGGSG | |
| | | | | | GGGSGGGGSEVQLQQSGPELVKPGASVKMS | |
| | | | | | CKASGYTFTNYVMNWVKQKPGQGLEWIGYI | |
| | | | | | NPYNDGTKYNERFKGKATLTSDKSSSTAFM | |
| | | | | | EVSSLTSEDSAVYYCARLYRGNAMDYWGQG | |
| | | | | | TSVTVSS | |
| 54 | 3A6 HCDR1 | HCDR1 | DNA | Artificial | CGTTATGGCATGTCT | |
| | (DNA) | | | sequence | | |
| 55 | 3A6 HCDR1 | HCDR1 | PRT | Artificial | RYGMS | |
| | | | | sequence | | |
| 56 | 3A6 HCDR2 | HCDR2 | DNA | Artificial | ACCATTATTAGTGGTGGTATTAACACCTAC | |
| | (DNA) | | | sequence | TATTCAGACAGTGTGAAGGGG | |
| 57 | 3A6 HCDR2 | HCDR2 | PRT | Artificial | THISGGINTYYSDSVKG | |
| | | | | sequence | | |
| 58 | 3A6 HCDR3 | HCDR3 | DNA | Artificial | CTCTACTATGGCAATGCTATGGACTAC | |
| | (DNA) | | | sequence | | |
| 59 | 3A6 HCDR3 | HCDR3 | PRT | Artificial | LYYGNAMDY | |
| | | | | sequence | | |
| 60 | 3A6 VH (DNA) | VH | DNA | Artificial | GAGGTGCAGTTGGTGGAGTCTGGGGGAGAC | |
| | | | | sequence | TTAGTGCAGCCTGGAGGGTCCCTGAAACTC | |
| | | | | | TCCTGTGCAGCCTCTGGATTCACTTTAAGT | |
| | | | | | CGTTATGGCATGTCTTGGGTTCGCCAGACT | |
| | | | | | CCTGACAAGAGGCTGGAGTGGGTCTCAACC | |
| | | | | | ATTATTAGTGGTGGTATTAACACCTACTAT | |
| | | | | | TCAGACAGTGTGAAGGGGCGATTCTCCATC | |
| | | | | | TCCAGAGACAATGCCAGGAACACCCTGTAC | |
| | | | | | CTGCAAATGAGCAGTCTGAAGTCTGAGGAC | |
| | | | | | ACAGCCATGTATTACTGTGGAAGACTCTAC | |
| | | | | | TATGGCAATGCTATGGACTACTGGGGGCAA | |
| | | | | | GGAACCGCTGTCACCGTCTCCTCA | |
| 61 | 3A6 VH | VH | PRT | Artificial | EVQLVESGGDLVQPGGSLKLSCAASGFTLS | |
| | | | | sequence | RYGMSWVRQTPDKRLEWVSTIISGGINTYY | |
| | | | | | SDSVKGRFSISRDNARNTLYLQMSSLKSED | |
| | | | | | TAMYYCGRLYYGNAMDYWGQGTAVTVSS | |
| 44 | 3A6 | LCDR1 | DNA | Artificial | AAGTCCAGTCAGAGTCTGTTAAACAGTGGA | |
| | LCDR1 | | | sequence | AATCAAAAGAACTACTTGACC | |
| | (DNA) | | | | | |
| 45 | 3A6 LCDR1 | LCDR1 | PRT | Artificial | KSSQSLLNSGNQKNYLT | |
| | | | | sequence | | |
| 46 | 3A6 LCDR2 | LCDR2 | DNA | Artificial | TGGGCATCCACTAGGGAATCT | |
| | (DNA) | | | sequence | | |
| 30 | 3A6 LCDR2 | LCDR2 | PRT | Artificial | WASTRES | |
| | | | | sequence | | |
| 62 | 3A6 LCDR3 | LCDR3 | DNA | Artificial | CAGAATGATTATAGITATCCTCTCACG | |
| | (DNA) | | | sequence | | |
| 63 | 3A6 LCDR3 | LCDR3 | PRT | Artificial | QNDYSYPLT | |
| | | | | sequence | | |
| 64 | 3A6 VL (DNA) | VL | DNA | Artificial | GACATTGTGATGACACAGTCTCCATCCTCC | |
| | | | | sequence | CTGACTGTGACAGCAGGAGAGAAGGTCACT | |
| | | | | | ATGAGCTGCAAGTCCAGTCAGAGTCTGTTA | |
| | | | | | AACAGTGGAAATCAAAAGAACTACTTGACC | |
| | | | | | TGGTACCAGCAGAAACCAGGGCAGCCTCCT | |
| | | | | | AAACTGTTGATCTACTGGGCATCCACTAGG | |
| | | | | | GAATCTGGGGTCCCTGATCGCTTCACAGGC | |
| | | | | | AGTGGATCTGGAACACATTTCACTCTCACC | |
| | | | | | ATCAGCAGTGTGCAGGCTGAAGACCTGGCA | |
| | | | | | GTTTATTACTGTCAGAATGATTATAGTTAT | |
| | | | | | CCTCTCACGTTCGGTGCTGGGACCAAGCTG | |
| | | | | | GAGCTGAAACGG | |
| 65 | 3A6 VL | VL | PRT | Artificial | DIVMTQSPSSLTVTAGEKVTMSCKSSQSLL | |
| | | | | sequence | NSGNQKNYLTWYQQKPGQPPKLLIYWASTR | |
| | | | | | ESGVPDRFTGSGSGTHETLTISSVQAEDLA | |
| | | | | | VYYCQNDYSYPLTFGAGTKLELKR | |
| 50 | Linker of | Linker | DNA | Artificial | GGCGGAGGAGGATCCGGAGGCGGAGGAAGC | |
| | 3A6 scFv | | | sequence | GGAGGAGGCGGATCT | |
| | (DNA) | | | | | |
| 51 | Linker of | Linker | PRT | Artificial | GGGGSGGGGSGGGGS | |
| | 3A6 scFv | | | sequence | | |
| 66 | 3A6 | scFv | DNA | Artificial | GACATTGTGATGACACAGTCTCCATCCTCCC | |
| | scFv | | | sequence | TGACTGTGACAGCAGGAGAGAAGGTCACTA | |
| | (DNA) | | | | TGAGCTGCAAGTCCAGTCAGAGTCTGTTAA | |
| | | | | | ACAGTGGAAATCAAAAGAACTACTTGACCT | |
| | | | | | GGTACCAGCAGAAACCAGGGCAGCCTCCTA | |
| | | | | | AACTGTTGATCTACTGGGCATCCACTAGGG | |
| | | | | | AATCTGGGGTCCCTGATCGCTTCACAGGCA | |
| | | | | | GTGGATCTGGAACACATTTCACTCTCACCA | |
| | | | | | TCAGCAGTGTGCAGGCTGAAGACCTGGCAG | |
| | | | | | TTTATTACTGTCAGAATGATTATAGTTATC | |
| | | | | | CTCTCACGTTCGGTGCTGGGACCAAGCTGG | |
| | | | | | AGCTGAAACGGGGCGGAGGAGGATCCGGAG | |
| | | | | | GCGGAGGAAGCGGAGGAGGCGGATCTGAGG | |
| | | | | | TGCAGTTGGTGGAGTCTGGGGGAGACTTAG | |
| | | | | | TGCAGCCTGGAGGGTCCCTGAAACTCTCCT | |
| | | | | | GTGCAGCCTCTGGATTCACTTTAAGTCGTT | |
| | | | | | ATGGCATGTCTTGGGTTCGCCAGACTCCTG | |
| | | | | | ACAAGAGGCTGGAGTGGGTCTCAACCATTA | |
| | | | | | TTAGTGGTGGTATTAACACCTACTATTCAG | |
| | | | | | ACAGTGTGAAGGGGCGATTCTCCATCTCCA | |
| | | | | | GAGACAATGCCAGGAACACCCTGTACCTGC | |
| | | | | | AAATGAGCAGTCTGAAGTCTGAGGACACAG | |
| | | | | | CCATGTATTACTGTGGAAGACTCTACTATG | |
| | | | | | GCAATGCTATGGACTACTGGGGGCAAGGAA | |
| | | | | | CCGCTGTCACCGTCTCCTCA | |
| 67 | 3A6 scFv | scFv | PRT | Artificial | DIVMTQSPSSLTVTAGEKVTMSCKSSQSLL | |
| | | | | sequence | NSGNQKNYLTWYQQKPGQPPKLLIYWASTR | |
| | | | | | ESGVPDRFTGSGSGTHFTLTISSVQAEDLA | |
| | | | | | VYYCQNDYSYPLTFGAGTKLELKRGGGGSG | |
| | | | | | GGGSGGGGSEVQLVESGGDLVQPGGSLKLS | |
| | | | | | CAASGFTLSRYGMSWVRQTPDKRLEWVSTI | |
| | | | | | ISGGINTYYSDSVKGRFSISRDNARNTLYL | |
| | | | | | QMSSLKSEDTAMYYCGRLYYGNAMDYWGQG | |
| | | | | | TAVTVSS | |
| 68 | 7E3 HCDR1 | HCDR1 | DNA | Artificial | AACTATATTATACAC | |
| | (DNA) | | | sequence | | |
| 69 | 7E3 HCDR1 | HCDR1 | PRT | Artificial | NYIIH | |
| | | | | sequence | | |
| 70 | 7E3 HCDR2 (DNA) | HCDR2 | DNA | Artificial | TATATTAATCCTTACACTGATGGTCCTAAG | |
| | | | | sequence | TACAATGAGAAGTTCAAAGGC | |
| 71 | 7E3 HCDR2 | HCDR2 | PRT | Artificial | YINPYTDGPKYNEKEKG | |
| | | | | sequence | | |
| 72 | 7E3 HCDR3 (DNA) | HCDR3 | DNA | Artificial | CTAAATAGAGGCAATGCTATGGACTAC | |
| | | | | sequence | | |
| 73 | 7E3 HCDR3 | HCDR3 | PRT | Artificial | LNRGNAMDY | |
| | | | | sequence | | |
| 74 | 7E3 VH | VH | DNA | Artificial | GAGGTCCAGTTGCAGCAGTCTGGACCTGAG | |
| | (DNA) | | | sequence | CTGGTAAAGCCTGGGGCTICAGTGAAGATG | |
| | | | | | TCCTGCAAGGCTTCTGGATACACATTCACT | |
| | | | | | AACTATATTATACACTGGGTGAAGCAGAAG | |
| | | | | | CCTGGGCAGGGCCTTGAGTGGATTGGATAT | |
| | | | | | ATTAATCCTTACACTGATGGTCCTAAGTAC | |
| | | | | | AATGAGAAGTTCAAAGGCAGGGCCACACTG | |
| | | | | | ACTTCAGACAAATCCTCCAGTACAGCCTAC | |
| | | | | | ATGGAGTTCAGCAGCCTGACCTCTGAGGAC | |
| | | | | | TCTGCGGTCTATTACTGTGCAAGACTAAAT | |
| | | | | | AGAGGCAATGCTATGGACTACTGGGGTCAA | |
| | | | | | GGAACCTCAGTCACCGTCTCCTCA | |
| 75 | 7E3 VH | VH | PRT | Artificial | EVQLQQSGPELVKPGASVKMSCKASGYTFT | |
| | | | | sequence | NYIIHWVKQKPGQGLEWIGYINPYTDGPKY | |
| | | | | | NEKFKGRATLTSDKSSSTAYMEFSSLTSED | |
| | | | | | SAVYYCARLNRGNAMDYWGQGTSVTVSS | |
| 44 | 7E3 LCDR1 (DNA) | LCDR1 | DNA | Artificial | AAGTCCAGTCAGAGTCTGITAAACAGTGGA | |
| | | | | sequence | AATCAAAAGAACTACTTGACC | |
| 45 | 7E3 LCDR1 | LCDR1 | PRT | Artificial | KSSQSLLNSGNQKNYLT | |
| | | | | sequence | | |
| 46 | 7E3 LCDR2 (DNA) | LCDR2 | DNA | Artificial | TGGGCATCCACTAGGGAATCT | |
| | | | | sequence | | |
| 30 | 7E3 LCDR2 | LCDR2 | PRT | Artificial | WASTRES | |
| | | | | sequence | | |
| 47 | 7E3 LCDR3 (DNA) | LCDR3 | DNA | Artificial | CAGAATGTTTATATTTATCCGTTCACG | |
| | | | | sequence | | |
| 32 | 7E3 LCDR3 | LCDR3 | PRT | Artificial | QNVYTYPET | |
| | | | | sequence | | |
| 48 | 7E3 VL (DNA) | VL | DNA | Artificial | GACATTGTGATGACACAGTCTCCATCCTCC | |
| | | | | sequence | CTGACTGTGACAGTAAGAGAGAAGGTCACT | |
| | | | | | TTGAGCTGCAAGTCCAGTCAGAGTCTGTTA | |
| | | | | | AACAGTGGAAATCAAAAGAACTACTTGACC | |
| | | | | | TGGTACCAGCAGAAACCAGGGCAGCCTCCT | |
| | | | | | AAACTTTTGATCTACTGGGCATCCACTAGG | |
| | | | | | GAATCTGGGGTCCCTGATCGCTTCACAGGC | |
| | | | | | AGTGGATCTGGCACAGATTTCACTCTCACC | |
| | | | | | ATCAGCAGTGTGCAGGCTGAAGACCTGGCA | |
| | | | | | GTTTATTACTGTCAGAATGTTTATATTTAT | |
| | | | | | CCGTTCACGTTCGGTGCTGGGACCAAGCTG | |
| | | | | | GAGCTGAAACGG | |
| 49 | 7E3 VL | VL | PRT | Artificial | DIVMTQSPSSLTVTVREKVTLSCKSSQSLL | |
| | | | | sequence | NSGNQKNYLTWYQQKPGQPPKLLIYWASTR | |
| | | | | | ESGVPDRFTGSGSGTDFTLTISSVQAEDLA | |
| | | | | | VYYCQNVYIYPFTFGAGTKLELKR | |
| 50 | Linker of 7E3 | Linker | DNA | Artificial | GGCGGAGGAGGATCCGGAGGCGGAGGAAGC | |
| | scFv (DNA) | | | sequence | GGAGGAGGCGGATCT | |
| 51 | Linker of | Linker | PR | Artificial | GGGGSGGGGSGGGGS | |
| | 7E3 scFv | | | sequence | | |
| 76 | 7E3 scFv | scFv | DNA | Artificial | GACATTGTGATGACACAGTCTCCATCCTCC | |
| | (DNA) | | | sequence | CTGACTGTGACAGTAAGAGAGAAGGTCACT | |
| | | | | | TTGAGCTGCAAGTCCAGTCAGAGTCTGTTA | |
| | | | | | AACAGTGGAAATCAAAAGAACTACTTGACC | |
| | | | | | TGGTACCAGCAGAAACCAGGGCAGCCTCCT | |
| | | | | | AAACTTTTGATCTACTGGGCATCCACTAGG | |
| | | | | | GAATCTGGGGTCCCTGATCGCTTCACAGGC | |
| | | | | | AGTGGATCTGGCACAGATTTCACTCTCACC | |
| | | | | | ATCAGCAGTGTGCAGGCTGAAGACCTGGCA | |
| | | | | | GTTTATTACTGTCAGAATGTTTATATTTAT | |
| | | | | | CCGTTCACGTTCGGTGCTGGGACCAAGCTG | |
| | | | | | GAGCTGAAACGGGGCGGAGGAGGATCCGGA | |
| | | | | | GGCGGAGGAAGCGGAGGAGGCGGATCTGAG | |
| | | | | | GTCCAGTTGCAGCAGTCTGGACCTGAGCTG | |
| | | | | | GTAAAGCCTGGGGCTTCAGTGAAGATGTCC | |
| | | | | | TGCAAGGCTTCTGGATACACATTCACTAAC | |
| | | | | | TATATTATACACTGGGTGAAGCAGAAGCCT | |
| | | | | | GGGCAGGGCCTTGAGTGGATTGGATATATT | |
| | | | | | AATCCTTACACTGATGGTCCTAAGTACAAT | |
| | | | | | GAGAAGTTCAAAGGCAGGGCCACACTGACT | |
| | | | | | TCAGACAAATCCTCCAGTACAGCCTACATG | |
| | | | | | GAGTTCAGCAGCCTGACCTCTGAGGACTCT | |
| | | | | | GCGGTCTATTACTGTGCAAGACTAAATAGA | |
| | | | | | GGCAATGCTATGGACTACTGGGGTCAAGGA | |
| | | | | | ACCTCAGTCACCGTCTCCTCA | |
| 77 | 7E3 scFv | scFv | PRT | Artificial | DIVMTQSPSSLTVTVREKVTLSCKSSQSLL | |
| | | | | sequence | NSGNQKNYLTWYQQKPGQPPKLLIYWASTR | |
| | | | | | ESGVPDRFTGSGSGTDFTLTISSVQAEDLA | |
| | | | | | VYYCQNVYIYPFTFGAGTKLELKRGGGGSG | |
| | | | | | GGGSGGGGSEVQLQQSGPELVKPGASVKMS | |
| | | | | | CKASGYTFTNYIIHWVKQKPGQGLEWIGYI | |
| | | | | | NPYTDGPKYNEKFKGRATLTSDKSSSTAYM | |
| | | | | | EFSSLTSEDSAVYYCARLNRGNAMDYWGQG | |
| | | | | | TSVTVSS | |
| 68 | 14E12 HCDR1 | HCDR1 | DNA | Artificial | AACTATATTATACAC | |
| | (DNA) | | | sequence | | |
| 69 | 14E12 HCDR1 | HCDR1 | PRT | Artificial | NYIIH | |
| | | | | sequence | | |
| 70 | 14E12 HCDR2 | HCDR2 | DNA | Artificial | TATATTAATCCTTACACTGATGGTCCTAAG | |
| | (DNA) | | | sequence | TACAATGAGAAGTTCAAAGGC | |
| 71 | 14E12 HCDR2 | HCDR2 | PRT | Artificial | YINPYTDGPKYNEKFKG | |
| | | | | sequence | | |
| 72 | 14E12 HCDR3 | HCDR3 | DNA | Artificial | CTAAATAGAGGCAATGCTATGGACTAC | |
| | (DNA) | | | sequence | | |
| 73 | 14E12 HCDR3 | HCDR3 | PRT | Artificial | LNRGNAMDY | |
| | | | | sequence | | |
| 78 | 14E12 VH (DNA) | VH | DNA | Artificial | GAGGTGCAGCTGCAGGAGTCTGGACCTGAG | |
| | | | | sequence | CTGGTAAAGCCTGGGGCTTCAGTGAAGATG | |
| | | | | | TCCTGCAAGGCTTCTGGATACACATTCACT | |
| | | | | | AACTATATTATACACTGGGTGAAGCAGAAG | |
| | | | | | CCTGGGCAGGGCCTTGAGTGGATTGGATAT | |
| | | | | | ATTAATCCTTACACTGATGGTCCTAAGTAC | |
| | | | | | AATGAGAAGTTCAAAGGCAGGGCCACACTG | |
| | | | | | ACTTCAGACAAATCCTCCAGTACAGCCTAC | |
| | | | | | ATGGAGTTCAGCAGCCTGACCTCTGAGGAC | |
| | | | | | TCTGCGGTCTATTACTGTGCAAGACTAAAT | |
| | | | | | AGAGGCAATGCTATGGACTACTGGGGTCAA | |
| | | | | | GGAACCTCAGTCACCGTCTCCTCA | |
| 79 | 14E12 VH | VH | PRT | Artificial | EVQLQESGPELVKPGASVKMSCKASGYTFT | |
| | | | | sequence | NYIIHWVKQKPGQGLEWIGYINPYTDGPKY | |
| | | | | | NEKFKGRATLTSDKSSSTAYMEFSSLTSED | |
| | | | | | SAVYYCARLNRGNAMDYWGQGTSVTVSS | |
| 80 | 14E12 LCDR1 | LCDR1 | DNA | Artificial | AAGTCCAGTCAGAGTCTGTTAAACAATGGA | |
| | (DNA) | | | sequence | AATCAAAAGAACTATTTGGCC | |
| 81 | 14E12 LCDR1 | LCDR1 | PRT | Artificial | KSSQSLLNNGNQKNYLA | |
| | | | | sequence | | |
| 82 | 14E12 LCDR2 | LCDR2 | DNA | Artificial | GGGGCATCCACTAGGGAATCT | |
| | (DNA) | | | sequence | | |
| 83 | 14E12 LCDR2 | LCDR2 | PRT | Artificial | GASTRES | |
| | | | | sequence | | |
| 84 | 14E12 LCDR3 | LCDR3 | DNA | Artificial | CAGAATGATCTTTATTATCCATTCACG | |
| | (DNA) | | | sequence | | |
| 85 | 14E12 LCDR3 | LCDR3 | PRT | Artificial | QNDLYYPFT | |
| | | | | sequence | | |
| 86 | 14E12 | VL | DNA | Artificial | GATATTGTGATGACCCAGTCTCCATCTTCC | |
| | VL (DNA) | | | sequence | CTGAGTGTGTCAGCAGGAGAGAAGGTCACT | |
| | | | | | ATGAGCTGCAAGTCCAGTCAGAGTCTGTTA | |
| | | | | | AACAATGGAAATCAAAAGAACTATTTGGCC | |
| | | | | | TGGTACCAGCAGAAACCAGGGCAGCCTCCT | |
| | | | | | AAACTGTTGATCTACGGGGCATCCACTAGG | |
| | | | | | GAATCTGGGGTCCCTGATCGCTTCACAGGC | |
| | | | | | AGTGGATCTGGAACCGATTTCGCTCTTACC | |
| | | | | | ATCAGCAGTGTGCAGGCTGAAGACCTGGCA | |
| | | | | | GTTTATTACTGTCAGAATGATCTTTATTAT | |
| | | | | | CCATTCACGTTCGGCTCGGGGACAAAGTTG | |
| | | | | | GACATAAAACGG | |
| 87 | 14E12 VL | VL | PRT | Artificial | DIVMTQSPSSLSVSAGEKVTMSCKSSQSLL | |
| | | | | sequence | NNGNQKNYLAWYQQKPGQPPKLLIYGASTR | |
| | | | | | ESGVPDRFTGSGSGTDFALTISSVQAEDLA | |
| | | | | | VYYCQNDLYYPFTFGSGTKLDIKR | |
| 50 | Linker of | Linker | DNA | Artificial | GGCGGAGGAGGATCCGGAGGCGGAGGAAGC | |
| | 14E12 | | | sequence | GGAGGAGGCGGATCT | |
| | scFv (DNA) | | | | | |
| 51 | Linker of | Linker | PRT | Artificial | GGGGSGGGGSGGGGS | |
| | 14E12 scFv | | | sequence | | |
| 88 | 14E12 scFv | scFv | DNA | Artificial | GATATTGTGATGACCCAGTCTCCATCTTCC | |
| | (DNA) | | | sequence | CTGAGTGTGTCAGCAGGAGAGAAGGTCACT | |
| | | | | | ATGAGCTGCAAGTCCAGTCAGAGTCTGTTA | |
| | | | | | AACAATGGAAATCAAAAGAACTATTTGGCC | |
| | | | | | TGGTACCAGCAGAAACCAGGGCAGCCTCCT | |
| | | | | | AAACTGTTGATCTACGGGGCATCCACTAGG | |
| | | | | | GAATCTGGGGTCCCTGATCGCTTCACAGGC | |
| | | | | | AGTGGATCTGGAACCGATTTCGCTCTTACC | |
| | | | | | ATCAGCAGTGTGCAGGCTGAAGACCTGGCA | |
| | | | | | GTTTATTACTGTCAGAATGATCTTTATTAT | |
| | | | | | CCATTCACGTTCGGCTCGGGGACAAAGTTG | |
| | | | | | GACATAAAACGGGGCGGAGGAGGATCCGGA | |
| | | | | | GGCGGAGGAAGCGGAGGAGGCGGATCTGAG | |
| | | | | | GTGCAGCTGCAGGAGTCTGGACCTGAGCTG | |
| | | | | | GTAAAGCCTGGGGCTTCAGTGAAGATGTCC | |
| | | | | | TGCAAGGCTTCTGGATACACATTCACTAAC | |
| | | | | | TATATTATACACTGGGTGAAGCAGAAGCCT | |
| | | | | | GGGCAGGGCCTTGAGTGGATTGGATATATT | |
| | | | | | AATCCTTACACTGATGGTCCTAAGTACAAT | |
| | | | | | GAGAAGTTCAAAGGCAGGGCCACACTGACT | |
| | | | | | TCAGACAAATCCTCCAGTACAGCCTACATG | |
| | | | | | GAGTTCAGCAGCCTGACCTCTGAGGACTCT | |
| | | | | | GCGGTCTATTACTGTGCAAGACTAAATAGA | |
| | | | | | GGCAATGCTATGGACTACTGGGGTCAAGGA | |
| | | | | | ACCTCAGTCACCGTCTCCTCA | |
| 89 | 14E12 scFv | scFv | PRT | Artificial | DIVMTQSPSSLSVSAGEKVTMSCKSSQSLL | |
| | | | | sequence | NNGNQKNYLAWYQQKPGQPPKLLIYGASTR | |
| | | | | | ESGVPDRFTGSGSGTDFALTISSVQAEDLA | |
| | | | | | VYYCQNDLYYPFTFGSGTKLDIKRGGGGSG | |
| | | | | | GGGSGGGGSEVQLQESGPELVKPGASVKMS | |
| | | | | | CKASGYTFTNYHIHWVKQKPGQGLEWIGYI | |
| | | | | | NPYTDGPKYNEKFKGRATLTSDKSSSTAYM | |
| | | | | | EFSSLTSEDSAVYYCARLNRGNAMDYWGQG | |
| | | | | | TSVTVSS | |
| 68 | 17B10 HCDR1 | HCDR1 | DNA | Artificial | AACTATATTATACAC | |
| | (DNA) | | | sequence | | |
| 69 | 17B10 HCDR1 | HCDR1 | PRT | Artificial | NYNIH | |
| | | | | sequence | | |
| 70 | 17B10 HCDR2 | HCDR2 | DNA | Artificial | TATATTAATCCTTACACTGATGGTCCTAAG | |
| | (DNA) | | | sequence | TACAATGAGAAGTTCAAAGGC | |
| 71 | 17B10 HCDR2 | HCDR2 | PRT | Artificial | YINPYTDGPKYNEKFKG | |
| | | | | sequence | | |
| 72 | 17B10 HCDR3 | HCDR3 | DNA | Artificial | CTAAATAGAGGCAATGCTATGGACTAC | |
| | (DNA) | | | sequence | | |
| 73 | 17B10 HCDR3 | HCDR3 | PRT | Artificial | LNRGNAMDY | |
| | | | | sequence | | |
| 78 | 17B10 VH (DNA) | VH | DNA | Artificial | GAGGTGCAGCTGCAGGAGTCTGGACCTGAG | |
| | | | | sequence | CTGGTAAAGCCTGGGGCTTCAGTGAAGATG | |
| | | | | | TCCTGCAAGGCTTCTGGATACACATTCACT | |
| | | | | | AACTATATTATACACTGGGTGAAGCAGAAG | |
| | | | | | CCTGGGCAGGGCCTTGAGTGGATTGGATAT | |
| | | | | | ATTAATCCTTACACTGATGGTCCTAAGTAC | |
| | | | | | AATGAGAAGTTCAAAGGCAGGGCCACACTG | |
| | | | | | ACTTCAGACAAATCCTCCAGTACAGCCTAC | |
| | | | | | ATGGAGTTCAGCAGCCTGACCTCTGAGGAC | |
| | | | | | TCTGCGGTCTATTACTGTGCAAGACTAAAT | |
| | | | | | AGAGGCAATGCTATGGACTACTGGGGTCAA | |
| | | | | | GGAACCTCAGTCACCGTCTCCTCA | |
| 79 | 17B10 VH | VH | PRT | Artificial | EVQLQESGPELVKPGASVKMSCKASGYTFT | |
| | | | | sequence | NYIIHWYKQKPGQGLEWIGYINPYTDGPKY | |
| | | | | | NEKFKGRATLTSDKSSSTAYMEFSSLTSED | |
| | | | | | SAVYYCARLNRGNAMDYWGQGTSVTVSS | |
| 44 | 17B10 LCDR1 | LCDR1 | DNA | Artificial | AAGTCCAGTCAGAGTCTGTTAAACAGTGGA | |
| | (DNA) | | | sequence | AATCAAAAGAACTACTTGACC | |
| 45 | 17B10 LCDR1 | LCDR1 | PRT | Artificial | KSSQSLLNSGNQKNYLT | |
| | | | | sequence | | |
| 46 | 17B10 LCDR2 | LCDR2 | DNA | Artificial | TGGGCATCCACTAGGGAATCT | |
| | (DNA) | | | sequence | | |
| 30 | 17B10 LCDR2 | LCDR2 | PRT | Artificial | WASTRES | |
| | | | | sequence | | |
| 90 | 17B10 LCDR3 | LCDR3 | DNA | Artificial | CAGAATGATTATAGTTTTCCATTCACG | |
| | (DNA) | | | sequence | | |
| 91 | 17B10 LCDR3 | LCDR3 | PRT | Artificial | QNDYSFPFT | |
| | | | | sequence | | |
| 92 | 17B10 VL (DNA) | VL | DNA | Artificial | GATATTGTGATGACCCAGTCTCCATCCTCC | |
| | | | | sequence | CTGACTGTGACAGCAGGAGAGAAGGTCACT | |
| | | | | | ATGAACTGCAAGTCCAGTCAGAGTCTGTTA | |
| | | | | | AACAGTGGAAATCAAAAGAACTACTTGACC | |
| | | | | | TGGTACCAGCAGAAACCAGGGCAGCCTCCT | |
| | | | | | AAACTGTTGATCTACTGGGCATCCACTAGG | |
| | | | | | GAATCTGGGGTCCCTGATCGGTTCACAGGC | |
| | | | | | AGTGGATCTGGAACAGATTTCACTCTCACC | |
| | | | | | ATCAGCAGTGTGCAGGCTGAAGACCTGGCA | |
| | | | | | GTTTATTACTGTCAGAATGATTATAGTTTT | |
| | | | | | CCATTCACGTTCGGCTCGGGGACAAAGTTG | |
| | | | | | GAAATAAAACGG | |
| 93 | 17B10 VL | VL | PRT | Artificial | DIVMTQSPSSLTVTAGEKVTMNCKSSQSLL | |
| | | | | sequence | NSGNQKNYLTWYQQKPGQPPKLLIYWASTR | |
| | | | | | ESGVPDRFTGSGSGTDETLTISSVQAEDLA | |
| | | | | | VYYCQNDYSFPFTFGSGTKLEIKR | |
| 50 | Linker of | Linker | DNA | Artificial | GGCGGAGGAGGATCCGGAGGCGGAGGAAGC | |
| | 17B10 | | | sequence | GGAGGAGGCGGATCT | |
| | scFv (DNA) | | | | | |
| 51 | Linker of | Linker | PRT | Artificial | GGGGSGGGGSGGGGS | |
| | 17B10 scFv | | | sequence | | |
| 94 | 17B10 scFv | scFv | DNA | Artificial | GATATTGTGATGACCCAGTCTCCATCCTCC | |
| | (DNA) of 7E3 | | | sequence | CTGACTGTGACAGCAGGAGAGAAGGTCACT | |
| | scFv (DNA) | | | | ATGAACTGCAAGTCCAGTCAGAGTCTGTTA | |
| | | | | | AACAGTGGAAATCAAAAGAACTACTTGACC | |
| | | | | | TGGTACCAGCAGAAACCAGGGCAGCCTCCT | |
| | | | | | AAACTGTTGATCTACTGGGCATCCACTAGG | |
| | | | | | GAATCTGGGGTCCCTGATCGGTTCACAGGC | |
| | | | | | AGTGGATCTGGAAGAGATTTCACTCTCACC | |
| | | | | | ATCAGCAGTGTGCAGGCTGAAGACCTGGCA | |
| | | | | | GTTTATTACTGTCAGAATGATTATAGTTTT | |
| | | | | | CCATTCACGTTCGGCTCGGGGACAAAGTTG | |
| | | | | | GAAATAAAACGGGGCGGAGGAGGATCCGGA | |
| | | | | | GGCGGAGGAAGCGGAGGAGGCGGATCTGAG | |
| | | | | | GTGCAGCTGCAGGAGTCTGGACCTGAGCTG | |
| | | | | | GTAAAGCCTGGGGCTTCAGTGAAGATGTCC | |
| | | | | | TGCAAGGCTTCTGGATACACATTCACTAAC | |
| | | | | | TATATTATACACTGGGTGAAGCAGAAGCCT | |
| | | | | | GGGCAGGGCCTTGAGTGGATTGGATATATT | |
| | | | | | AATCCTTACACTGATGGTCCTAAGTACAAT | |
| | | | | | GAGAAGTTCAAAGGCAGGGCCACACTGACT | |
| | | | | | TCAGACAAATCCTCCAGTACAGCCTACATG | |
| | | | | | GAGTTCAGCAGCCTGACCTCTGAGGACTCT | |
| | | | | | GCGGTCTATTACTGTGCAAGACTAAATAGA | |
| | | | | | GGCAATGCTATGGACTACTGGGGTCAAGGA | |
| | | | | | ACCTCAGTCACCGTCTCCTCA | |
| 95 | 17B10 scFv | scFv | PRT | Artificial | DIVMTQSPSSLTVTAGEKVTMNCKSSQSLL | |
| | | | | sequence | NSGNQKNYLTWYQQKPGQPPKLLIYWASTR | |
| | | | | | ESGVPDRFTGSGSGTDETLTISSVQAEDLA | |
| | | | | | VYYCQNDYSFPFTFGSGTKLEIKRGGGGSG | |
| | | | | | GGGSGGGGSEVQLQESGPELVKPGASVKMS | |
| | | | | | CKASGYTFTNYIIHWVKQKPGQGLEWIGYI | |
| | | | | | NPYTDGPKYNEKFKGRATLTSDKSSSTAYM | |
| | | | | | EFSSLTSEDSAVYYCARLNRGNAMDYWGQG | |
| | | | | | TSVTVSS | |
| 96 | MSLN3 HCDR1 | HCDR1 | DNA | Artificial | cgctataacatgggc | |
| | (DNA) | | | sequence | | |
| 97 | MSLN3 HCDR1 | HCDR1 | PRT | Artificial | RYNMG | |
| | | | | sequence | | |
| 98 | MSLN3 HCDR2 | HCDR2 | DNA | Artificial | tatattagctggcgcggtggcagcacctat | |
| | (DNA) | | | sequence | tacccagatagcgtgganggc | |
| 99 | MSLN3 HCDR2 | HCDR2 | PRT | Artificial | YISWRGGSTYYPDSVEG | |
| | | | | sequence | | |
| 100 | MSLN3 HCDR3 | HCDR3 | DNA | Artificial | agcatcaaaaccgccgatccagatgattac | |
| | (DNA) | | | sequence | gattac | |
| 101 | MSLN3 HCDR3 | HCDR3 | PRT | Artificial | SIKTADPDDYDY | |
| | | | | sequence | | |
| 102 | MSLN3 VHH (DNA) | VHH | DNA | Artificial | gaggtgcagctggtggagagcggtggtggt | |
| | | | | sequence | ctcgtgcagcccggcggtagtctgcgcctc | |
| | | | | | agctgtgccgccagcggtaacacccttagc | |
| | | | | | cgctataacatgggctggtttcgccaagcc | |
| | | | | | cccggcaaaggtcgcgaactggtggcctat | |
| | | | | | attagctggcgcggtggcagcacctattac | |
| | | | | | ccagatagcgtggaaggccgcttcaccatc | |
| | | | | | agccgcgataacgccaagcgcatggtgtat | |
| | | | | | ctgcagatgaacagtctgcgcgccgaggac | |
| | | | | | accgccgtgtattattgtgccgccagcatc | |
| | | | | | aaaaccgccgatccagatgattacgattac | |
| | | | | | tggggccaaggcacccaagtgaccgtgagc | |
| | | | | | agc | |
| 103 | MSLN3 | VHH | PRT | Artificial | EVQLVESGGGLVQPGGSLRLSCAASGNTLS | |
| | VHH | | | sequence | RYNMGWFRQAPGKGRELVAYISWRGGSTYY | |
| | | | | | PDSVEGRETISRDNAKRMVYLQMNSLRAED | |
| | | | | | TAVYYCAASIKTADPDDYDYWGQGTQVTVS | |
| | | | | | S | |
| 104 | A06 HCDR1 | HCDR1 | PRT | Artificial | DYYMG | |
| | | | | sequence | | |
| 105 | A06 HCDR2 | HCDR2 | PRT | Artificial | GITSSGSDTLYPDSVEG | |
| | | | | sequence | | |
| 106 | A06 HCDR3 | HCDR3 | PRT | Artificial | TYQGNRPVRTDQFLGGYNY | |
| | | | | sequence | | |
| 107 | A06 VHH | VHH | PRT | Artificial | EVQLVESGGGLVQPGGSLRISCAASGIIFS | |
| | | | | sequence | DYYMGWFRQAPGKGRELVAGITSSGSDTLY | |
| | | | | | PDSVEGRFTISRDNAKRMVYLQMNSLRAED | |
| | | | | | TAVYYCAATYQGNRPVRTDQFLGGYNYWGQ | |
| | | | | | GTLVTVSS | |
| 108 | SS1 HCDR1 | HCDR1 | PRT | Artificial | GYTMN | |
| | | | | sequence | | |
| 109 | SS1 HCDR2 | HCDR2 | PRT | Artificial | LITPYNGASSYNQKFRG | |
| | | | | sequence | | |
| 110 | SS1 HCDR3 | HCDR3 | PRT | Artificial | GGYDGRGEDY | |
| | | | | sequence | | |
| | SS1 VH | VH | PRT | Artificial | QVQLQQSGPELEKPGASVKLSCKASGYSFT | |
| | | | | sequence | GYTMNWVKQSHGKSLEWIGLITPYNGASSY | |
| | | | | | NQKFRGKATLTVDKSSSTAYMDLLSLTSED | |
| | | | | | SAVYFCARGGYDGRGFDYWGQGTTVTVSS | |
| 112 | SS1 LCDR1 | LCDR | PRT | Artificial | SASSSVSYMH | |
| | | | | sequence | | |
| 113 | SS1 LCDR2 | LCDR2 | PRT | Artificial | DTSKLAS | |
| | | | | sequence | | |
| 114 | SS1 LCDR3 | LCDR3 | PRT | Artificial | QQWSGYPLT | |
| | | | | sequence | | |
| 115 | SS1 VL | VL | PRT | Artificial | DIELTQSPAIMSASPGEKVTMTCSASSSVS | |
| | | | | sequence | YMHWYQQKSGTSPKRWIYDTSKLASGVPGR | |
| | | | | | FSGSGSGNSYSLTISSVEAEDDATYYCQQW | |
| | | | | | SGYPLTFGAGTKLEIKR | |
| 116 | SS1 scFv | scFv | PRT | Artificial | QVQLQQSGPELEKPGASVKLSCKASGYSFT | |
| | | | | sequence | GYTMNWVKQSHGKSLEWIGLITPYNGASSY | |
| | | | | | NQKFRGKATLTVDKSSSTAYMDLLSLTSED | |
| | | | | | SAVYFCARGGYDGRGFDYWGQGTTVTVSSG | |
| | | | | | VGGSGGGGSGGGGSDIELTQSPAIMSASPG | |
| | | | | | EKVTMTCSASSSVSYMHWYQQKSGTSPKRW | |
| | | | | | IYDTSKLASGVPGRFSGSGSGNSYSLTISS | |
| | | | | | VEAEDDATYYCQQWSGYPLTEGAGTKLEIK | |
| | | | | | R | |
| 117 | YP218 HCDR1 | HCDR1 | PRT | Artificial | FYFYAC | |
| | | | | sequence | | |
| 118 | YP218 HCDR2 | HCDR2 | PRT | Artificial | CIYTAGSGSTYYASWAKG | |
| | | | | sequence | | |
| 119 | YP218 HCDR3 | HCDR3 | PRT | Artificial | STANTRSTYYLNL | |
| | | | | sequence | | |
| 120 | YP218 VH | VH | PRT | Artificial | QQQLEESGGGLVKPEGSLTLTCKASGFDLG | |
| | | | | sequence | FYFYACWVRQAPGKGLEWIACIYTAGSGST | |
| | | | | | YYASWAKGRFTISKASSTTVTLQMTSLAAA | |
| | | | | | DTATYFCARSTANTRSTYYLNLWGPGTLVT | |
| | | | | | VSS | |
| 121 | YP218 LCDR1 | LCDR1 | PRT | Artificial | QASQRISSYLS | |
| | | | | sequence | | |
| 122 | YP218 LCDR2 | LCDR2 | PRT | Artificial | GASTLAS | |
| | | | | sequence | | |
| 123 | YP218 LCDR3 | LCDR3 | PRT | Artificial | QSYAYFDSNNWHA | |
| | | | | sequence | | |
| 124 | YP218 VL | VL | PRT | Artificial | DVVMTQTPASVSEPVGGTVTIKCQASQRIS | |
| | | | | sequence | SYLSWYQQKPGQRPKLLIFGASTLASGVPS | |
| | | | | | RFKGSGSGTEYTLTISDLECADAATYYCQS | |
| | | | | | YAYFDSNNWHAFGGGTEVVV | |
| 125 | YP218 scFv | scFv | PRT | Artificial | QQQLEESGGGLVKPEGSLTLTCKASGFDLG | |
| | | | | sequence | FYFYACWVRQAPGKGLEWIACIYTAGSGST | |
| | | | | | YYASWAKGRETISKASSTTVTLQMTSLAAA | |
| | | | | | DTATYFCARSTANTRSTYYLNLWGPGTLVT | |
| | | | | | VSSGGGGSGGGGSGGGGSDVVMTQTPASVS | |
| | | | | | EPVGGTVTIKCQASQRISSYLSWYQQKPGQ | |
| | | | | | RPKLLIFGASTLASGVPSRFKGSGSGTEYT | |
| | | | | | LTISDLECADAATYYCQSYAYFDSNNWHAF | |
| | | | | | GGGTEVVV | |
| 126 | Standard HCDR1 | HCDR1 | DNA | Artificial | AGCGGCTACAACTGGCAC | |
| | (DNA) | | | sequence | | |
| 127 | Standard HCDR1 | HCDR1 | PRT | Artificial | SGYNWH | |
| | | | | sequence | | |
| 128 | Standard | HCDR2 | DNA | Artificial | TACATCCACTACACCGGCAGCACCAACTAC | |
| | HCDR2 | | | sequence | AACCCCGCCCTGAGAAGC | |
| | (DNA) | | | | | |
| 129 | Standard HCDR2 | HCDR2 | PRT | Artificial | YIHYTGSTNYNPALRS | |
| | | | | sequence | | |
| 130 | Standard HCDR3 | HCDR3 | DNA | Artificial | ATCTACAACGGCAACAGCTTCCCTTAT | |
| | (DNA) | | | sequence | | |
| 131 | Standard HCDR3 | HCDR3 | PRT | Artificial | TYNGNSFPY | |
| | | | | sequence | | |
| 132 | Standard VH | VH | DNA | Artificial | CAGGTGCAACTACAGGAGAGCGGCCCCGGT | |
| | (DNA) | | | sequence | CTGATCAAGCCCAGCCAGACCCTGAGCCTG | |
| | | | | | ACCTGCACCGTGAGCGGCGGCAGCATCAGC | |
| | | | | | AGCGGCTACAACTGGCACTGGATCAGACAG | |
| | | | | | CCCCCCGGCAAGGGCCTGGAGTGGATCQGC | |
| | | | | | TACATCCACTACACCGGCAGCACCAACTAC | |
| | | | | | AACCCCGCCCTGAGAAGCAGAGTGACCATC | |
| | | | | | AGCGTGGACACCAGCAAGAACCAGTTCAGC | |
| | | | | | CTGAAGCTGAGCAGCGTGACCGCCGCCGAC | |
| | | | | | ACCGCCATCTACTACTGCGCCAGAATCTAC | |
| | | | | | AACGGCAACAGCTTCCCTTATTGGGGCCAG | |
| | | | | | GGCACCACCGTGACCGTGAGCAGC | |
| 133 | Standard VH | VH | PRT | Artificial | QVQLQESGPGLIKPSQTLSLTCTVSGGSIS | |
| | | | | sequence | SGYNWHWIRQPPGKGLEWIGYIHYTGSTNY | |
| | | | | | NPALRSRVTISVDTSKNQFSLKLSSVTAAD | |
| | | | | | TAIYYCARIYNGNSFPYWGQGTTVTVSS | |
| 134 | Standard LCDR1 | LCDR1 | DNA | Artificial | AAGAGCAGCCAGAGCCTGTTCAACAGCGGC | |
| | (DNA) | | | sequence | AACCAGAAGAACTACCTGACC | |
| 135 | Standard LCDR1 | LCDR1 | PRT | Artificial | KSSQSLFNSGNQKNYLT | |
| | | | | sequence | | |
| 136 | Standard LCDR2 | LCDR2 | DNA | Artificial | TGGGCCAGCACCAGAGAGAGC | |
| | (DNA) | | | sequence | | |
| 30 | Standard LCDR2 | LCDR2 | PRI | Artificial | WASTRES | |
| | | | | sequence | | |
| 137 | Standard LCDR3 | LCDR3 | DNA | Artificial | CAGAACGCCTATAGCTTTCCCTATACA | |
| | (DNA) | | | sequence | | |
| 138 | Standard LCDR3 | LCDR3 | PRT | Artificial | QNAYSFPYT | |
| | | | | sequence | | |
| 139 | Standard VL | VL | DNA | Artificial | GACATCGTGATGACCCAGAGCCCCGACAGC | |
| | (DNA) | | | sequence | CTGGCCGTGAGCCTGGGCGAGAGAGCCACC | |
| | | | | | ATCAACTGCAAGAGCAGCCAGAGCCTGTTC | |
| | | | | | AACAGCGGCAACCAGAAGAACTACCTGACC | |
| | | | | | TGGTACCAGCAGAAGCCCGGCCAGCCCCCC | |
| | | | | | AAGCTGCTGATCTACTGGGCCAGCACCAGA | |
| | | | | | GAGAGCGGCGTGCCCGACAGATTCAGCGGC | |
| | | | | | AGCGGCAGCGGCACCGACTTCACCCTGACC | |
| | | | | | ATCAGCAGCCTGCAGGCCGAGGACGTGGCC | |
| | | | | | GTGTACTACTGCCAGAACGCCTATAGCTTT | |
| | | | | | CCCTATACATTTGGCGGTGGGACCAAGCTG | |
| | | | | | GAGATCAAGCGG | |
| 140 | Standard VL | VL | PRT | Artificial | DIVMTQSPDSLAVSLGERATINCKSSQSLE | |
| | | | | sequence | NSGNQKNYLTWYQQKPGQPPKLLIYWASTR | |
| | | | | | ESGVPDRFSGSGSGTDFTITISSLQAEDVA | |
| | | | | | VYYCQNAYSFPYTFGGGTKLEIKR | |
| 141 | Linker of | Linker | DNA | Artificial | GGTGGGGGGGGCTCTGGTGGTGGAGGCTCC | |
| | standard scFv | | | sequence | GGAGGCGGTGGGAGC | |
| | (DNA) | | | | | |
| 51 | Linker of | Linker | PRT | Artificial | GGGGSGGGGSGGGGS | |
| | standard scFv | | | sequence | | |
| 142 | Standard scFv | scFv | DNA | Artificial | GACATCGTGATGACCCAGAGCCCCGACAGC | |
| | (DNA) | | | sequence | CTGGCCGTGAGCCTGGGCGAGAGAGCCACC | |
| | | | | | ATCAACTGçAAGAGCAGCCAGAGCCTGTTC | |
| | | | | | AACAGCGGCAACCAGAAGAACTACCTGACC | |
| | | | | | TGGTACCAGCAGAAGCCCGGCCAGCCCCCC | |
| | | | | | AAGCTGCTGATCTACTGGGCCAGCACCAGA | |
| | | | | | GAGAGCGGCGTGCCCGACAGATTCAGCGGC | |
| | | | | | AGCGGCAGCGGCACCGACTTCACCCTGACC | |
| | | | | | ATCAGCAGCCTGCAGGCCGAGGACGTGGCC | |
| | | | | | GTGTACTACTGCCAGAACGCCTATAGCTTT | |
| | | | | | CCCTATACATTTGGCGGTGGGACCAAGCTG | |
| | | | | | GAGATCAAGCGGGGTGGGGGGGGCTCTGGT | |
| | | | | | GGTGGAGGCTCCGGAGGCGGTGGGAGCCAG | |
| | | | | | GTGCAACTACAGGAGAGCGGCCCCGGTCTG | |
| | | | | | ATCAAGCCCAGCCAGACCCTGAGCCTGACC | |
| | | | | | TGCACCGTGAGCGGCGGCAGCATCAGCAGC | |
| | | | | | GGCTACAACTGGCACTGGATCAGACAGCCC | |
| | | | | | CCCGGCAAGGGCCTGGAGTGGATCGGCTAC | |
| | | | | | ATCCACTACACCGGCAGCACCAACTACAAC | |
| | | | | | CCCGCCCTGAGAAGCAGAGTGACCATCAGC | |
| | | | | | GTGGACACCAGCAAGAACCAGTTCAGCCTG | |
| | | | | | AAGCTGAGCAGCGTGACCGCCGCCGACACC | |
| | | | | | GCCATCTACTACTGCGCCAGAATCTACAAC | |
| | | | | | GGCAACAGCTTCCCTTATTGGGGCCAGGGC | |
| | | | | | ACCACCGTGACCGTGAGCAGC | |
| 143 | Standard scFv | scFv | PRT | Artificial | DIVMTQSPDSLAVSLGERATINCKSSQSLF | |
| | | | | sequence | NSGNQKNYLTWYQQKPGQPPKLLIYWASTR | |
| | | | | | ESGVPDRFSGSGSGTDFTLTISSLQAEDVA | |
| | | | | | VYYCQNAYSFPYTEGGGTKLEIKRGGGGSG | |
| | | | | | GGGSGGGGSQVQLQESGPGLIKPSQTLSLT | |
| | | | | | CTVSGGSISSGYNWHWIRQPPGKGLEWIGY | |
| | | | | | IHYTGSTNYNPALRSRVTISVDTSKNQFSL | |
| | | | | | KLSSVTAADTAIYYCARIYNGNSFPYWGQG | |
| | | | | | TTVTVSS | |
| 144 | CLDN1 8.2 HCDR3 | 5E6 HCDR3: | PRT | Artificial | L[NY][RY]GNAMDY | |
| | general formula | 5E5 HCDR3: | | sequence | | |
| | | 3A6 HCDR3: | | | | |
| | | 7E3 HCDR3: | | | | |
| | | 14E12 HCDR3: | | | | |
| | | 17B10 HCDR3 | | | | |
| 145 | CLDN1 8.2 HCDR2 | 5E6 HCDR2: | PRT | Artificial | [IY]I[IN][PS][GY][GNT][DI][GN] | |
| | general formula | 5E5 HCDR2: | | sequence | [PT][KY]Y[NS][DE][KRS][FV]KG | |
| | | 3A6 HCDR2: | | | | |
| | | 7E3 HCDR2: | | | | |
| | | 14E12 HCDR2: | | | | |
| | | 17B10 HCDR2 | | | | |
| 146 | CLDN1 8.2 HCDR1 | 5E6 HCDR1: | PRT | Artificial | [NR]Y[GIV][IM][HNS] | |
| | general formula | 5E5 HCDR1: | | sequence | | |
| | | 3A6 HCDR1: | | | | |
| | | 7E3 HCDR1: | | | | |
| | | 14E12 HCDR1: | | | | |
| | | 7B10 HCDR1 | | | | |
| 147 | CLDN1 8.2 VH | 5E6 VH: | PRT | Artificial | [EQ]VQL[QV][EQ]SG[AGP][DE][LV] | |
| | general formula | 5E5 VH: | | sequence | [KV][KQ]PG[AG]S[LV]K[LMV]DC[AK] | |
| | | 3A6 VH: | | | [ASG][FY]T[F][ST][NR]Y[GIV][IM | |
| | | 7E3 VH: | | | ][HNS]WV[KR]Q[AKT]P[DG][KQ][GR] | |
| | | 14E12 VH: | | | [LEW][IMV][GS][TY]I[IN][PS][GY] | |
| | | 17B10 VH | | | [GNT][DI][GN][PT][KY]Y[NS][DE] | |
| | | | | | [KRS][FV]KG[KR][AFV][ST][IL] | |
| | | | | | [ST][RS]D[KNT][AS][ARS][NS]T[A | |
| | | | | | L][FY][LM][EQ][FLMV]SSL[KRT]SE | |
| | | | | | D[ST]A[MV]YYC[AG]RL[NY][RY]GNA | |
| | | | | | MDYWGQGT[ALS]VTVSS | |
| 148 | CLDN1 8.2 LCDR3 | 5E6 LCDR3: | PRT | Artificial | QN[DV][LY][ISY][FY]P[FL]T | |
| | general formula | 5E5 LCDR3: | | sequence | | |
| | | 7E3 LCDR3: | | | | |
| | | 3A6 LCDR3: | | | | |
| | | 14E12 LCDR3: | | | | |
| | | 17B10 LCDR3 | | | | |
| 149 | CLDN1 8.2 LCDR2 | 5E6 LCDR2: | PRT | Artificial | [GW]ASTRES | |
| | general formula | 5E5 LCDR2: | | sequence | | |
| | | 3A6 LCDR2: | | | | |
| | | 7E3 LCDR2: | | | | |
| | | 17B10 LCDR2: | | | | |
| | | Standard LCDR2: | | | | |
| | | 14E12 LCDR2 | | | | |
| 150 | CLDN1 8.2 LCDR1 | 5E6 LCDR1: | PRT | Artificial | KSSQSLL[AN][NS]GNQKNYL[AT] | |
| | general formula | 5E5 LCDR1: | | sequence | | |
| | | 3A6 LCDR1: | | | | |
| | | 7E3 LCDR1: | | | | |
| | | 17B10 LCDR1: | | | | |
| | | 14E12 LCDR1 | | | | |
| 151 | CLDN1 8.2 VL | 5E6 VL: | PRT | Artificial | DIVMTQSP[DS]SL[AST]V[ST][ALV][ | |
| | general formula | SES VL: | | sequence | GR]E[KR][AV]T[ILM][NS]CKSSQSLL | |
| | | 7E3 VL: | | | [AN][NS]GNQKNYL[AT]WYQQKPGQPPK | |
| | | 3A6 VL: | | | LLIY[GW]ASTRESGVPDRF[ST]GSGSGT | |
| | | 14E12 VL: | | | [DH]F[AT]LTISS[LV]QAED[LV]AVYY | |
| | | 17B10 VL | | | CQN[DV][LY][ISY][FY]P[FL]TEG[A | |
| | | | | | QS]GTKL[DE][IL]KR | |
| 152 | MSLN5 2 HCDR1 | MSLN5 | PRT | Artificial | YYPKA | |
| | | 2 HCDR1 | | sequence | | |
| 153 | MSLN5 2 HCDR2 | MSLN5 | PRT | Artificial | SIGWGGRMTAYADSVKG | |
| | | HCDR2 | | sequence | | |
| 154 | MSLNS 2 HCDR3 | MSLN5 2 | PRT | Artificial | GIGWAPTADSGEYDY | |
| | | HCDR3 | | sequence | | |
| 155 | MSLN5 2 VHH | MSLN5 2 | PRT | Artificial | EVQLLESGGGLVQPGGSLRLSCAASGHTDS | |
| | | VHH | | sequence | YYPKAWFRQAPGKEREFVASIGWGGRMTAY | |
| | | | | | ADSVKGRFTISRDNSKNTLYLQMNSLRAED | |
| | | | | | TAVYYCAAGIGWAPTADSGEYDYWGQGTLV | |
| | | | | | TVSS | |
| 156 | MSLN5 2 VHH | MSLN5 | DNA | Artificial | GAGGTGCAGCTCCTGGAATCCGGCGGGGGC | |
| | (DNA) | 2 VHH (DNA) | | sequence | CTGGTGCAGCCCGGCGGGAGCCTCAGACTG | |
| | | | | | AGCTGCGCTGCTAGCGGCCACACCGACAGC | |
| | | | | | TACTACCCCAAGGCCTGGTTCAGACAAGCC | |
| | | | | | CCCGGCAAGGAGAGAGAGTTCGTGGCTAGC | |
| | | | | | ATTGGCTGGGGCGGCAGAATGACCGCCTAC | |
| | | | | | GCCGACAGCGTGAAGGGCAGATTCACCATC | |
| | | | | | AGCAGAGACAACAGCAAGAACACCCTGTAC | |
| | | | | | CTGCAGATGAACAGCCTGAGAGCCGAGGAC | |
| | | | | | ACAGCCGTGTACTACTGTGCCGCCGGCATC | |
| | | | | | GGCTGGGCCCCTACAGCCGACAGCGGCGAG | |
| | | | | | TACGACTACTGGGGCCAAGGCACCCTGGTG | |
| | | | | | ACCGTGAGCAGC | |
| 157 | Zolbetuximab | Zolbetuximab | PRT | Artificial | QVQLQQPGAELVRPGASVKLSCKASGYTFT | |
| | heavy chain | heavy chain | | sequence | SYWINWVKQRPGQGLEWIGNIYPSDSYTNY | |
| | | | | | NQKFKDKATLTVDKSSSTAYMQLSSPTSED | |
| | | | | | SAVYYCTRSWRGNSFDYWGQGTTLTVSSAS | |
| | | | | | TKGPSVFPLAPSSKSTSGGTAALGCLVKDY | |
| | | | | | FPEPVTVSWNSGALTSGVHTFPAVLQSSGL | |
| | | | | | YSLSSVVTVPSSSLGTQTYICNVNHKPSNT | |
| | | | | | KVDKRVEPKSCDKTHTCPPCPAPELLGGPS | |
| | | | | | VFLFPPKPKDTLMISRTPEVTCVVVDVSHE | |
| | | | | | DPEVKFNWYVDGVEVHNAKTKPREEQYNST | |
| | | | | | YRVVSVLTVLHQDWLNGKEYKCKVSNKALP | |
| | | | | | APIEKTISKAKGQPREPQVYTLPPSREEMT | |
| | | | | | KNQVSLTCLVKGFYPSDIAVEWESNGQPEN | |
| | | | | | NYKTTPPVLDSDGSFFLYSKLTVDKSRWQQ | |
| | | | | | GNVESCSVMHEALHNHYTQKSLSLSPGK | |
| 158 | Zolbetuximab | Zolbetuximab | PRT | Artificial | DIVMTQSPSSLTVTAGEKVTMSCKSSQSLL | |
| | light chain | light chain | | sequence | NSGNQKNYLTWYQQKPGQPPKLLIYWASTR | |
| | | | | | ESGVPDRFTGSGSGTDFTLTISSVQAEDLA | |
| | | | | | VYYCQNDYSYPFTFGSGTKLEIKRTVAAPS | |
| | | | | | VFIFPPSDEQLKSGTASVVCLLNNFYPREA | |
| | | | | | KVQWKVDNALQSGNSQESVTEQDSKDSTYS | |
| | | | | | LSSTLTLSKADYEKHKVYACEVTHQGLSSP | |
| | | | | | VTKSFNRGEC | |

1. A cell, comprising and/or expressing a chimeric antigen receptor targeting claudin 18.2 (CLDN18.2), and a chimeric antigen receptor targeting mesothelin (MSLN) protein, wherein said chimeric antigen receptor targeting CLDN18.2 comprises an antigen binding domain targeting CLDN18.2, said chimeric antigen receptor targeting MSLN comprises an antigen binding domain targeting MSLN.

2. (canceled)

3. (canceled)

4. The cell of claim 1, wherein said antigen binding domain targeting CLDN18.2 comprises HCDR3, HCDR2, HCDR1, LCDR3, LCDR2, and LCDR1, and said HCDR3 comprises an amino acid sequence as set forth in SEQ ID NO: 144, said HCDR2 comprises an amino acid sequence as set forth in SEQ ID NO: 145, said HCDR1 comprises an amino acid sequence as set forth in SEQ ID NO: 146 (X₁YX₂×₃×₄, wherein X₁ is N or R, X₂ is G, I or V, X₃ is I or M, and X₄ is H, N or S), said LCDR3 comprises an amino acid sequence as set forth in SEQ ID NO: 148, said LCDR2 comprises an amino acid sequence as set forth in SEQ ID NO: 149, and said LCDR1 comprises an amino acid sequence as set forth in SEQ ID NO: 150.

5. The cell of claim 1, wherein said antigen binding domain targeting CLDN18.2 comprises an HCDR3, HCDR2, HCDR1, LCDR3, LCDR2, and LCDR1, wherein said HCDR3 comprises an amino acid sequence as set forth in any one of SEQ ID NO: 24, SEQ ID NO: 59, and SEQ ID NO: 73, said HCDR2 comprises an amino acid sequence as set forth in any one of SEQ ID NO: 22, SEQ ID NO: 57, and SEQ ID NO: 71, and said HCDR1 comprises an amino acid sequence as set forth in any one of SEQ ID NO: 20, SEQ ID NO: 55, and SEQ ID NO: 69, said LCDR3 comprises an amino acid sequence as set forth in any one of SEQ ID NO: 32, SEQ ID NO: 63, SEQ ID NO: 85, and SEQ ID NO: 91, said LCDR2 comprises an amino acid sequence as set forth in SEQ ID NO: 30 or SEQ ID NO: 83, and said LCDR1 comprises an amino acid sequence as set forth in any one of SEQ ID NO: 28, SEQ ID NO: 45, and SEQ ID NO: 81.

6-10. (canceled)

11. The cell of claim 1, wherein said antigen binding domain targeting CLDN18.2 comprises a VH and VL, and said VH comprises an amino acid sequence as set forth in any one of SEQ ID NO: 147, SEQ ID NO: 26, SEQ ID NO: 43, SEQ ID NO: 61, SEQ ID NO: 75, and SEQ ID NO: 79, and said VL comprises an amino acid sequence as set forth in any one of SEQ ID NO: 151, SEQ ID NO: 34, SEQ ID NO: 49, SEQ ID NO: 65, SEQ ID NO: 87, and SEQ ID NO: 93.

12-24. (canceled)

25. The cell of claim 1, wherein said antigen binding domain targeting CLDN18.2 comprises an scFv, said scFv comprises an amino acid sequence as set forth in any one of SEQ ID NO: 38, SEQ ID NO: 53, SEQ ID NO: 67, SEQ ID NO: 77, SEQ ID NO: 89, and SEQ ID NO: 95.

26. (canceled)

27. The cell of claim 1, wherein said chimeric antigen receptor targeting CLDN18.2 comprises a costimulatory domain, transmembrane region, hinge region, and/or signal peptide, and/or wherein said chimeric antigen receptor targeting CLDN18.2 does not comprise an intracellular signaling domain, said costimulatory domain comprises a costimulatory domain derived from one or more proteins selected from a group consisting of: CD28, 4-1BB, CD27, CD2, CD7, CD8, OX40, CD226, DR3, SLAM, CDS, ICAM-1, NKG2D, NKG2C, B7-H3, 2B4, FcεRIγ, BTLA, GITR, HVEM, DAP10, DAP12, CD30, CD40, CD40L, TIM1, PD-1, LFA-1, LIGHT, JAML, CD244, CD100, ICOS, a ligand of CD83, CD40, and MyD88, said transmembrane region comprises a transmembrane region derived from one or more proteins selected from a group consisting of: CD8, CD28, 4-1BB, CD4, CD27, CD7, PD-1, TRAC, TRBC, CD3ε, CD3ζ, CTLA-4, LAG-3, CD5, ICOS, OX40, NKG2D, 2B4, CD244, FcεRIγ, BTLA, CD30, GITR, HVEM, DAP10, CD2, NKG2C, LIGHT, DAP12, CD40L, TIM1, CD226, DR3, CD45, CD80, CD86, CD9, CD16, CD22, CD33, CD37, CD64, CD134, CD137, CD154, and SLAM, said hinge region comprises a hinge region derived from one or more proteins selected from a group consisting of: CD28, IgG1, IgG4, IgD, 4-1BB, CD4, CD27, CD7, CD8, PD-1, ICOS, OX40, NKG2D, NKG2C, FcεRIγ, BTLA, GITR, DAP10, CD40L, TIM1, CD226, SLAM, CD30, and LIGHT, said signal peptide is derived from a signal peptide of CD8 protein.

28-43. (canceled)

44. The cell of claim 1, wherein said antigen binding domain targeting MSLN comprises HCDR3, HCDR2, HCDR1, LCDR3, LCDR2, and LCDR1, and said HCDR3 comprises an amino acid sequence as set forth in any one of SEQ ID NO: 101, SEQ ID NO: 106, SEQ ID NO: 110, SEQ ID NO: 119, and SEQ ID NO: 154, said HCDR2 comprises an amino acid sequence as set forth in any one of SEQ ID NO: 99, SEQ ID NO: 105, SEQ ID NO: 109, SEQ ID NO: 118, and SEQ ID NO: 153, said HCDR1 comprises an amino acid sequence as set forth in any one of SEQ ID NO: 97, SEQ ID NO: 104, SEQ ID NO: 108, SEQ ID NO: 117, and SEQ ID NO: 152, said LCDR3 comprises an amino acid sequence as set forth in SEQ ID NO: 123 or SEQ ID NO: 114, said LCDR2 comprises an amino acid sequence as set forth in SEQ ID NO: 113 or SEQ ID NO: 122, and said LCDR1 comprises an amino acid sequence as set forth in SEQ ID NO: 112 or SEQ ID NO: 121.

45.-47. (canceled)

48. The cell of claim 1, wherein said antigen binding domain targeting MSLN comprises a VH and VL, and said VH comprises an amino acid sequence as set forth in any one of SEQ ID NO: 103, SEQ ID NO: 107, SEQ ID NO: 111, SEQ ID NO: 120, and SEQ ID NO: 155, and said VL comprises an amino acid sequence as set forth in SEQ ID NO: 115 or SEQ ID NO: 124.

49. The cell of claim 1, wherein said antigen binding domain targeting MSLN comprises a VHH, said VHH comprises HCDR3, HCDR2, and HCDR1, wherein said HCDR3 comprises an amino acid sequence as set forth in any one of SEQ ID NO: 101, SEQ ID NO: 106, SEQ ID NO: 110, SEQ ID NO: 119, and SEQ ID NO: 154, said HCDR2 comprises an amino acid sequence as set forth in any one of SEQ ID NO: 99, SEQ ID NO: 105, SEQ ID NO: 109, SEQ ID NO: 118, and SEQ ID NO: 153, and said HCDR1 comprises an amino acid sequence as set forth in any one of SEQ ID NO: 97, SEQ ID NO: 104, SEQ ID NO: 108, SEQ ID NO: 117, and SEQ ID NO: 152.

50. The cell of claim 49, wherein said VHH comprises an amino acid sequence as set forth in any one of SEQ ID NO: 103, SEQ ID NO: 107, and SEQ ID NO: 155.

51.-55. (canceled)

56. The cell of claim 1, wherein said antigen binding domain targeting MSLN comprises an scFv, and said scFv comprises an amino acid sequence as set forth in SEQ ID NO: 116 or SEQ ID NO: 125.

57. The cell of claim 1, wherein said chimeric antigen receptor targeting MSLN comprises an intracellular signaling domain, transmembrane region, hinge region, signal peptide, and/or low-density lipoprotein receptor-related protein or a fragment thereof, and/or wherein said chimeric antigen receptor targeting MSLN does not comprise a costimulatory domain, wherein and said intracellular signaling domain comprises an intracellular signaling domain derived from one or more proteins selected from a group consisting of: CD3ζ, CD3δ, CD3γ, CD3ε, CD79a, CD79b, FcεRIγ, FcεRIβ, FcγRIIa, bovine leukemia virus gp30, Epstein-Barr virus (EBV) LMP2A, simian immunodeficiency virus PBj14 Nef, Kaposi's sarcoma-associated herpesvirus (HSKV), DAP10, DAP-12, and a domain containing at least one ITAM, said transmembrane region comprises a transmembrane region derived from one or more proteins selected from a group consisting of: CD8, CD28, 4-1BB, CD4, CD27, CD7, PD-1, TRAC, TRBC, CD3ε, CD3ζ, CTLA-4, LAG-3, CD5, ICOS, OX40, NKG2D, 2B4, CD244, FcεRIγ, BTLA, CD30, GITR, HVEM, DAP10, CD2, NKG2C, LIGHT, DAP12, CD40L, TIM1, CD226, DR3, CD45, CD80, CD86, CD9, CD16, CD22, CD33, CD37, CD64, CD134, CD137, CD154, and SLAM, said hinge region comprises a hinge region derived from one or more proteins selected from a group consisting of: CD28, IgG1, IgG4, IgD, 4-1BB, CD4, CD27, CD7, CD8, PD-1, ICOS, OX40, NKG2D, NKG2C, FcεRIγ, BTLA, GITR, DAP10, CD40L, TIM1, CD226, SLAM, CD30, and LIGHT, said signal peptide is derived from a signal peptide of CD8 protein, said low-density lipoprotein receptor-related protein or the fragment thereof comprises one or more selected from a group consisting of: low-density lipoprotein receptor-related proteins 1-12 and functional fragments thereof.

58.-73. (canceled)

74. The cell of claim 1, wherein the expression level of said chimeric antigen receptor targeting CLDN18.2 is approximately 1:1 or approximately 2:1 with that of the chimeric antigen receptor targeting MSLN, said cell further comprising and/or expressing a low-density lipoprotein receptor-related protein or a fragment thereof, said low-density lipoprotein receptor-related protein or the fragment thereof comprises one or more selected from a group consisting of: low-density lipoprotein receptor-related proteins 1-12 and functional fragments thereof.

75-79. (canceled)

80. The cell of claim 1, comprising an immune effector cell, said cell comprising T cells, B cells, natural killer cells (NK cells), macrophages, NKT cells, monocytes, dendritic cells, granulocytes, lymphocytes, leukocytes, peripheral blood mononuclear cells, embryonic stem cells, lymphoid progenitor cells and/or pluripotent stem cells.

81-82. (canceled)

83. An expression vector, comprising a nucleic acid sequence which encodes a chimeric antigen receptor targeting CLDN18.2 and a nucleic acid sequence which encodes a chimeric antigen receptor targeting MSLN.

84-86. (canceled)

87. The expression vector of claim 83, wherein said nucleic acid sequence which encodes the chimeric antigen receptor targeting CLDN18.2 comprises a nucleic acid sequence which encodes an antigen binding domain targeting CLDN18.2, said antigen binding domain targeting CLDN18.2 comprises HCDR3, HCDR2, HCDR1, LCDR3, LCDR2 and LCDR1, and a nucleic acid sequence which encodes said HCDR3 comprises a nucleic acid sequence as set forth in any one of SEQ ID NO: 23, SEQ ID NO: 41, SEQ ID NO: 58, and SEQ ID NO: 72, a nucleic acid sequence which encodes said HCDR2 comprises a nucleic acid sequence as set forth in any one of SEQ ID NO: 21, SEQ ID NO: 40, SEQ ID NO: 56, and SEQ ID NO: 70, a nucleic acid sequence which encodes said HCDR1 comprises a nucleic acid sequence as set forth in any one of SEQ ID NO: 19, SEQ ID NO: 39, SEQ ID NO: 54, and SEQ ID NO: 68, a nucleic acid sequence which encodes said LCDR3 comprises a nucleic acid sequence as set forth in any one of SEQ ID NO: 31, SEQ ID NO: 47, SEQ ID NO: 62, SEQ ID NO: 84, and SEQ ID NO: 90, a nucleic acid sequence which encodes said LCDR2 comprises a nucleic acid sequence as set forth in any one of SEQ ID NO: 29, SEQ ID NO: 46, and SEQ ID NO: 82, and a nucleic acid sequence which encodes said LCDR1 comprises a nucleic acid sequence as set forth in any one of SEQ ID NO: 27, SEQ ID NO: 44, and SEQ ID NO: 80.

88-112. (canceled)

113. The expression vector of claim 83, wherein said nucleic acid sequence which encodes the chimeric antigen receptor targeting MSLN comprises a nucleic acid sequence which encodes an antigen binding domain targeting MSLN, wherein said antigen binding domain targeting MSLN comprises HCDR3, HCDR2, and HCDR1, and said HCDR3 comprises an amino acid sequence as set forth in any one of any one of SEQ ID NO: 101, SEQ ID NO: 106, SEQ ID NO: 110, SEQ ID NO: 119, and SEQ ID NO: 154, said HCDR2 comprises an amino acid sequence as set forth in any one of SEQ ID NO: 99, SEQ ID NO: 105, SEQ ID NO: 109, SEQ ID NO: 118, and SEQ ID NO: 153, and said HCDR1 comprises an amino acid sequence as set forth in any one of SEQ ID NO: 97, SEQ ID NO: 104, SEQ ID NO: 108, SEQ ID NO: 117, and SEQ ID NO: 152.

114-122. (canceled)

123. The expression vector of claim 83, wherein said antigen binding domain targeting MSLN comprises HCDR3, HCDR2, HCDR1, LCDR3, LCDR2 and LCDR1, and said HCDR3 comprises an amino acid sequence as set forth in any one of any one of SEQ ID NO: 101, SEQ ID NO: 106, SEQ ID NO: 110, SEQ ID NO: 119, and SEQ ID NO: 154, said HCDR2 comprises an amino acid sequence as set forth in any one of SEQ ID NO: 99, SEQ ID NO: 105, SEQ ID NO: 109, SEQ ID NO: 118, and SEQ ID NO: 153, said HCDR1 comprises an amino acid sequence as set forth in any one of SEQ ID NO: 97, SEQ ID NO: 104, SEQ ID NO: 108, SEQ ID NO: 117, and SEQ ID NO: 152, said LCDR3 comprises an amino acid sequence as set forth in SEQ ID NO: 114 or SEQ ID NO: 123, said LCDR2 comprises an amino acid sequence as set forth in SEQ ID NO: 113 or SEQ ID NO: 122, and said LCDR1 comprises an amino acid sequence as set forth in SEQ ID NO: 112 or SEQ ID NO: 121.

124-165. (canceled)

166. A cell, comprising the expression vector of claim 83.

167-175. (canceled)

176. A method for preventing and/or treating a disease and/or a disorder, comprising administering to a subject in need thereof the cell of claim 1 wherein said disease and/or disorder comprises a tumor, said tumor comprises a tumor simultaneously expressing both antigens CLDN18.2 and MSLN.

177-179. (canceled)

180. The method of claim 176, wherein said tumor comprises gastric cancer, pancreatic cancer, and/or gastroesophageal junction carcinoma.
